(19) United States
(12) Patent Application Publication
(10) Pub. No.: US 2007/0026409 A1
(43) Pub. Date: Feb. 1, 2007

(54) Nucleic acid and amino acid sequences involved in pain

(75) Inventors: Clifford Woolf, Newton, MA (US); Donatella D'Urso, Duesseldorf (DE); Katia Befort, Strasbourg (FR); Michael Costigan, Somerville, MA (US)

Correspondence Address: PALMER & DODGE, LLP;KATHLEEN M. WILLIAMS, 111 HUNTINGTON AVENUE, BOSTON, MA, 02199, US

(73) Assignee: The General Hospital Corporation

(21) Appl. No.: 10/743,643

(22) Filed: Dec. 22, 2003

Related U.S. Application Data
(63) Continuation-in-part of application No. 10/219,051, filed on Aug. 14, 2002.
(60) Provisional application No. 60/312,147, filed on Aug. 14, 2001.
(60) Provisional application No. 60/346,382, filed on Nov. 1, 2001.
(60) Provisional application No. 60/333,347, filed on Nov. 26, 2001.

(51) Int. Cl. C12Q 1/68; C07H 21/04; C07K 14/705; C12N 5/08

(52) U.S. Cl. 435/6; 435/69.1; 435/320.1; 435/368; 530/350; 536/23.5

(57) ABSTRACT

The present invention relates to nucleic acid sequences which are related to pain and which are differentially expressed during pain. The invention further relates to methods of identifying nucleic acid sequences which are differentially expressed during pain, microarrays comprising such differentially expressed sequences and methods of screening agents for the ability to regulate the expression of such differentially expressed sequences.

### PRIORITY

This application is a Continuation-in-Part of U.S. application Ser. No. 10/219,051, filed Aug. 14, 2002, which claims the benefit of U.S. Provisional Application No. 60/312,147, filed Aug. 14, 2001; U.S. Provisional Application No. 60/346,382, filed Nov. 1, 2001; and U.S. Provisional Application No. 60/333,347, filed Nov. 26, 2001. The entire teachings of the above applications are incorporated herein by reference.

### Sequence Listing

The present application includes a Sequence Listing submitted herewith on three identical CD-ROM disks pursuant to 37 C.F.R. §1.53(e). The information on each CD-ROM is identical. Submitted are the Computer Readable Copy (disk 1) of the sequence listing, and Copy 1 (disk 2) and Copy 2 (disk 3). The following information is identical for each CD-ROM submitted: Machine Format: IBM-PC; Operating System: MS-Windows; Files Contained: Formal_sequence_listing2ndappl.txt; Size: 7.56 MB; Date of Creation: Dec. 19, 2003. The information on each CD-ROM is incorporated herein by reference in its entirety.

### BACKGROUND OF THE INVENTION

Pain is a state-dependent sensory experience which can be represented by a constellation of distinct types of pain including chronic pain, neuropathic pain, inflammatory pain, and physiological pain. Current therapy is, however, either relatively ineffective or accompanies by substantial side effects (Sindrup and Jensen, 1999 Pain 83: 389). All of the primary forms of pain therapy have been discovered wither empirically through folk medicine, or serendipitously. These forms of treatment include opiates, non-steroidal anti-inflammatory drugs (NSAIDS), local anesthetics, anticonvulsants, and tricyclic antidepressants (TCAs).

Recently there has been a great deal of progress in understanding the mechanisms that produce pain (McCleskey and Gold, 1999, Annu. Rev. Physiol. 61: 835; Woolf and Salter, 2000, Science 288: 1765; Mogil et al., 2000, Annu. Rev. Neurosci. 23: 777). It is increasingly clear that multiple mechanisms operating at different sites, and with different temporal profiles, are involved. In consequence, there is a need in the art for a shift in pain management from treatment essentially by trial and error to a strategy that attempts to identify and treat the mechanisms present in a given patient (Woolf and Mannion, 1999, Lancet 353: 1959; Woolf and Decosterd, 1999, Pain 82: 1). Accordingly, there is a need in the art for techniques which enable the identification of the genes responsible for these mechanisms.

The present invention, in an effort to meet such a need, provides a plurality of genes which are differentially expressed in animals which have been subjected to pain. The present invention provides advantages over existing measurements of differential expression in that the invention provides lower thresholds of differential expression. The present invention thus encompasses a much larger number of genes which show differential expression, and therefore provides a much improved method for identifying a larger number of genes whose expression may be directly related to the mechanisms which underlie pain.

### SUMMARY OF THE INVENTION

The present invention provides a composition comprising two or more isolated polynucleotides, wherein each of said two or more isolated polynucleotides is selected from the polynucleotides of Table 11.

The present invention also provides a composition comprising two or more isolated polynucleotides, wherein each of said two or more isolated polynucleotides is selected from the polynucleotides of Table 10.

The invention also provides a composition comprising two or more isolated polynucleotides, wherein each of said two or more isolated polynucleotides is selected from the group consisting of: (a) a polynucleotide comprising any of the polynucleotides specified in Table 11 in the columns designated “rat gene” and “human gene”; (b) a polynucleotide encoding an amino acid sequence selected from the group consisting of: amino acid sequences which are homologous to any of the amino acid sequences specified in Table 11 in the columns designated “rat protein” and “human protein” by at least the homology as specified for the respective sequence in Table 11 in the column designated “% homology” and which encodes a polypeptide exhibiting the biological function as specified for the respective sequence in Table 11 in the column designated “identifier”; (c) the amino acid specified in Table 11 in the columns designated “rat protein” and “human protein”; (d) a polynucleotide which hybridizes under high stringency conditions to a polynucleotide specified in (a) to (b) and encodes a polypeptide exhibiting the biological function as specified for the respective sequence in Table 11 in the column designated “identifier”; (e) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 11 in the column designated “identifier”; and (f) a polynucleotide which represents a fragment, derivative or allelic variation of a nucleic acid sequence specified in (a) to (d) and encodes a polypeptide exhibiting the biological function as specified for the respective sequence in Table 11 in the column designated “identifier”.

The invention further provides polypeptide sequences, indicated by Accession no. in Table 11, which are encoded by the polynucleotide sequences shown in Table 11 which are differentially expressed by at least 1.2 fold across at least three replicate screens of neuronal tissue obtained from an animal subjected to pain relative to an animal not subjected to the same pain, with a P-value of less than 0.05.

The invention further provides polypeptide sequences, indicated by Accession no. in Table 11, which are encoded by the polynucleotide sequences shown in Table 11 which are differentially expressed by at least 1.4 fold across at least three replicate screens of neuronal tissue obtained from an animal subjected to pain relative to an animal not subjected to the same pain, with a P-value of less than 0.05.

The invention further provides human polypeptide sequences, indicated by Accession no. in Table 11, which are encoded by the human polynucleotide sequences shown in Table 11 which are differentially expressed by at least 1.2 fold across at least three replicate screens of neuronal tissue obtained from an animal subjected to pain relative to an animal not subjected to the same pain, with a P-value of less than 0.05.

The invention further provides polypeptide sequences, indicated by Accession no. in Table 11, which are encoded by the polynucleotide sequences shown in Table 11 which are differentially expressed by at least 1.4 fold in an animal subjected to pain relative to an animal not subjected to the same pain.

The invention further provides human polypeptide sequences, indicated by Accession no. in Table 11, which are encoded by the human polynucleotide sequences shown in Table 11 which are differentially expressed by at least 1.4 fold in an animal subjected to pain relative to an animal not subjected to the same pain.

The invention further provides human polynucleotide sequences, indicated by Accession no. in Table 11 which are differentially expressed by greater than 1.4 fold in an animal subjected to pain relative to an animal not subjected to pain and polypeptide sequences encoded thereby. Preferably, the animal is a human.

The invention further provides human polynucleotide sequences, indicated by Accession no. in Table 11, which are differentially expressed by at least 1.2 fold across at least three replicate screens of neuronal tissue obtained from an animal subjected to pain relative to an animal not subjected to the same pain, with a p-value of less than 0.05.

The invention further provides polypeptide sequences, indicated by Accession no. in Table 10, which are encoded by the polynucleotide sequences shown in Table 10 which are differentially expressed by at least 1.2 fold across at least three replicate screens of neuronal tissue obtained from an animal subjected to pain relative to an animal not subjected to the same pain, with a P-value of less than 0.05.

The invention further provides polypeptide sequences, indicated by Accession no. in Table 10, which are encoded by the polynucleotide sequences shown in Table 10 which are differentially expressed by at least 1.4 fold across at least three replicate screens of neuronal tissue obtained from an animal subjected to pain relative to an animal not subjected to the same pain, with a P-value of less than 0.05.

The invention further provides human polypeptide sequences, indicated by Accession no. in Table 10, which are encoded by the human polynucleotide sequences shown in Table 10 which are differentially expressed by at least 1.2 fold across at least three replicate screens of neuronal tissue obtained from an animal subjected to pain relative to an animal not subjected to the same pain, with a P-value of less than 0.05.

The invention further provides polypeptide sequences, indicated by Accession no. in Table 10, which are encoded by the polynucleotide sequences shown in Table 10 which are differentially expressed by at least 1.4 fold in an animal subjected to pain relative to an animal not subjected to the same pain.

The invention further provides human polypeptide sequences, indicated by Accession no. in Table 10, which are encoded by the human polynucleotide sequences shown in Table 10 which are differentially expressed by at least 1.4 fold in an animal subjected to pain relative to an animal not subjected to the same pain.

The invention further provides human polynucleotide sequences, indicated by Accession no. in Table 10 which are differentially expressed by greater than 1.4 fold in an animal subjected to pain relative to an animal not subjected to pain and polypeptide sequences encoded thereby. Preferably, the animal is a human.

The invention further provides human polynucleotide sequences, indicated by Accession no. in Table 10, which are differentially expressed by at least 1.2 fold across at least three replicate screens of neuronal tissue obtained from an animal subjected to pain relative to an animal not subjected to the same pain, with a p-value of less than 0.05.

The present invention provides a composition comprising polynucleotides which are differentially expressed by at least +/−1.2 fold in at least three replicate assays of nerve tissue obtained from a nerve injury or inflammation pain model, with a p-value of less than 0.05, wherein each of the polynucleotides is selected from the polynucleotides listed in Tables 10, and preferably selected from the polynucleotides listed in Table 11.

In one embodiment, each of the two or more isolated polynucleotides is differentially expressed by at least 1.4 fold in the nerve tissue of an animal subjected to pain relative to the animal not subjected to the pain, and alternatively, are differentially expressed by at least 1.4 fold across three replicate assays of expression in nerve tissue obtained from a nerve injury pain model with a p-value of less than 0.05.

In an alternate embodiment, each of the two or more isolated polynucleotides is differentially expressed by at least 2 fold in the neurons of an animal subjected to pain relative to the animal not subjected to the pain.

In one embodiment, the nerve tissue is the sensory neurons of the dorsal root ganglion, or dorsal horn of the spinal cord.

The present invention provides a composition comprising two or more isolated polynucleotides, wherein each of said two or more isolated polynucleotides is selected from the polynucleotides of Tables 1 or 2 or a sequence which hybridizes under high stringency conditions thereto, and wherein at least one of said two or more isolated polynucleotides is unique to Table 2, or a sequence which hybridizes under high stringency conditions thereto.

The invention also provides a composition comprising two or more isolated polynucleotides, wherein each of said two or more isolated polynucleotides is selected from the group consisting of: a polynucleotide comprising any of the polynucleotides specified in Table 1 or 2 in the columns designated “rat gene” and “human gene”, and wherein at least one of said two or more isolated polynucleotides is unique to Table 2 in the columns designated “rat gene” and “human gene”; a polynucleotide encoding an amino acid sequence selected from the group consisting of: amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; and the amino acid specified in Table 2 in the columns designated “rat protein” and “human protein”; a polynucleotide which hybridizes under high stringency conditions to a polynucleotide specified in (a) to (b) and encodes a polypeptide exhibiting the biological function as specified for the respective sequence in Table 2 in the column designated “identifier”; a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; and a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”.

The invention further provides polypeptide sequences, indicated by Accession no. in Table 2, which are encoded by the polynucleotide sequences shown in Tables 2 which are differentially expressed by at least 1.2 fold across at least three replicate screens of neuronal tissue obtained from an animal subjected to pain relative to an animal not subjected to the same pain, with a P-value of less than 0.05.

The invention further provides human polypeptide sequences, indicated by Accession no. in Table 2, which are encoded by the human polynucleotide sequences shown in Tables 2 which are differentially expressed by at least 1.2 fold across at least three replicate screens of neuronal tissue obtained from an animal subjected to pain relative to an animal not subjected to the same pain, with a P-value of less than 0.05.

The invention further provides polypeptide sequences, indicated by Accession no. in Tables 2 or 3, which are encoded by the polynucleotide sequences shown in Tables 2 or 3 which are differentially expressed by at least 1.4 fold in an animal subjected to pain relative to an animal not subjected to the same pain.

The invention further provides human polypeptide sequences, indicated by Accession no. in Tables 2 or 3, which are encoded by the human polynucleotide sequences shown in Tables 2 or 3 which are differentially expressed by at least 1.4 fold in an animal subjected to pain relative to an animal not subjected to the same pain.

The invention further provides human polynucleotide sequences, indicated by Accession no. in Table 2 or 3 which are differentially expressed by greater than 1.4 fold in an animal subjected to pain relative to an animal not subjected to pain and polypeptide sequences encoded thereby. Preferably, the animal is a human.

The invention further provides human polynucleotide sequences, indicated by Accession no. in Table 2, which are differentially expressed by at least 1.2 fold across at least three replicate screens of neuronal tissue obtained from an animal subjected to pain relative to an animal not subjected to the same pain, with a p-value of less than 0.05.

Table 1 of the present invention includes polynucleotide sequences which have been examined using the methods described herein, and have been previously individually described in the art as being regulated in animal models of pain. Not all of the polynucleotides shown in Table 1, however, are “differentially expressed” according to the present invention. The invention is based, in part, upon the discovery that certain polynucleotides shown in Table 1 are differentially expressed in nerve tissue. Those polynucleotides indicated as having a Fold change of +/−1.4 or greater are differentially expressed.

Table 2 and 3 of the present invention include polynucleotide sequences which have not been previously described in the art as being regulated in animal pain models and which have been analyzed in at least three replicate screens of neuronal tissue from animals subjected to pain, and have attained a statistical significance of p<0.05. Table 2 and 3, however, also include one or more of the sequence indicated in Table 1. Accordingly, the phrase “unique to Table x” refers to a sequence which is indicated in Table x, and is not indicated in Table 1. Therefore, the invention also is based, in part, upon the discovery that polynucleotides (listed in Tables 2 and 3) are differentially expressed in nerve tissue obtained from an animal subjected to pain relative to an animal not subjected to the same pain. This discovery is demonstrated in nerve injury models of pain: e.g., spared nerve injury, axotomy, chronic constriction, and nerve ligation, and inflammation pain models. Each of tables 2 and 3 represents a polynucleotide sequence which is identified herein as being differentially expressed in an animal subjected to pain by at least 1.4 fold relative to the expression of the same sequence in an animal which has not been subjected to the same pain. Table 2 represents sequences which have been analyzed in at least three replicate assays of differential expression and are differentially expressed by at least 1.4 fold in an animal subjected to pain relative to an animal not subjected to pain, and have a statistical significance of P<0.05. Thus, each of the polynucleotides shown in Tables 2 or 3 is differentially expressed in an animal subjected to pain according to the present invention.

Table 4 and 5 of the present invention include polynucleotide sequences which have not been previously described in the art as being regulated in an animal pain model, and which have been identified herein as being differentially expressed in an animal subjected to inflammatory pain by at least 1.4 fold. All of the sequences in Tables 4 and 5 are identified herein as being differentially expressed, and a number of the polynucleotides indicated in Tables 4 and 5 have also been included in Table 2, as having attained a statistical significance of p<0.05 in three replicate analyses of gene expression.

Accordingly, the present invention provides a composition comprising polynucleotides which are differentially expressed by at least +/−1.2 fold in at least three replicate assays of nerve tissue obtained from a nerve injury or inflammation pain model, with a p-value of less than 0.05, wherein each of the polynucleotides is selected from the polynucleotides listed in Tables 1 or 2, and wherein at least one of the polynucleotides is selected from the polynucleotides listed in Table 2.

In one embodiment, each of the two or more isolated polynucleotides is differentially expressed by at least 1.4 fold in the nerve tissue of an animal subjected to pain relative to the animal not subjected to the pain, and alternatively, are differentially expressed by at least 1.4 fold across three replicate assays of expression in nerve tissue obtained from a nerve injury pain model with a p-value of less than 0.05.

In an alternate embodiment, each of the two or more isolated polynucleotides is differentially expressed by at least 2 fold in the neurons of an animal subjected to pain relative to the animal not subjected to the pain.

In one embodiment, the nerve tissue is the sensory neurons of the dorsal root ganglion, or dorsal horn of the spinal cord.

The invention also provides a plurality of vectors each comprising an isolated polynucleotide, wherein each of the isolated polynucleotides is selected from Table 1, 2, 3, 4, or 5, or a sequence which hybridizes under high stringency conditions thereto, and wherein at least one of the isolated polynucleotides is unique to Table 2, 3, 4, or 5, or a sequence which hybridizes under high stringency conditions thereto.

The invention also provides a plurality of vectors each comprising an isolated polynucleotide, wherein each of the isolated polynucleotides is selected from Table 10 and/or 11, or a sequence which hybridizes under high stringency conditions thereto.

The invention further provides a plurality of viral vectors each comprising an isolated polynucleotide, wherein each of the isolated polynucleotides is selected from Table 1, 2, 3, 4, or 5, or a sequence which hybridizes under high stringency conditions thereto, and wherein at least one of the isolated polynucleotides is unique to Table 2, 3, 4, or 5 or a sequence which hybridizes under high stringency conditions thereto.

The invention further provides a plurality of viral vectors each comprising an isolated polynucleotide, wherein each of the isolated polynucleotides is selected from Table 10 and/or 11, or a sequence which hybridizes under high stringency conditions thereto.

The invention further provides a plurality of vectors each comprising an isolated polynucleotide, wherein each of said two or more isolated polynucleotides is selected from the group consisting of: (a) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (b) a polynucleotide encoding an amino acid sequence selected from the group consisting of: (i) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (ii) the amino acid specified in Table 2 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 2 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”.

The invention further provides a plurality of vectors each comprising an isolated polynucleotide, wherein each of said two or more isolated polynucleotides is selected from the group consisting of: (a) a polynucleotide comprising any of the polynucleotides specified in Table 10 and/or 11 in the columns designated “rat gene” and “human gene”; (b) a polynucleotide encoding an amino acid sequence selected from the group consisting of: (i) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 or 11 in the column designated “identifier”; (ii) the amino acid specified in Table 10 or 11 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 10 or 11 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 10 or 11 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 10 or 11 in the column designated “identifier”.

In one embodiment, the vectors described above are contained within a host cell.

The invention further provides a method for identifying a nucleotide sequence which is differentially regulated in an animal subjected to pain, comprising: hybridizing a nucleic acid sample corresponding to RNA obtained from the animal to at least three replicates of a nucleic acid sample comprising one or more nucleic acid molecules of known identity; measuring the hybridization of the nucleic acid sample to the one or more nucleic acid molecules of known identity for each of the replicates, wherein a 1.2 fold difference in the hybridization, and a p-value of less than 0.05 across the at least three replicates, of the nucleic acid sample to the one or more nucleic acid molecules of known identity relative to a nucleic acid sample obtained from an animal which has not been subjected to the pain is indicative of the differential expression of the nucleotide sequence in the animal subjected to pain.

The present invention also provides a method for identifying a nucleotide sequence which is differentially regulated in an animal subjected to pain, comprising: hybridizing a nucleic acid sample corresponding to RNA obtained from the animal to a nucleic acid sample comprising one or more nucleic acid molecules of known identity; measuring the hybridization of the nucleic acid sample to the one or more nucleic acid molecules of known identity, wherein a 1.4 fold difference in the hybridization of the nucleic acid sample to the one or more nucleic acid molecules of known identity relative to a nucleic acid sample obtained from an animal which has not been subjected to the pain is indicative of the differential expression of the nucleotide sequence in the animal subjected to pain.

The invention further provides a method for identifying a nucleotide sequence which is differentially regulated in an animal subjected to pain, comprising: hybridizing a nucleic acid sample corresponding to RNA obtained from the animal to at least three replicates of an array comprising a solid substrate and one or more nucleic acid molecules of known identity; wherein each nucleic acid member has a unique position and is stably associated with the solid substrate; and measuring the hybridization of the nucleic acid sample to the at least three replicates of the array, wherein a 1.2 fold difference in the hybridization, and a p-value of less than 0.05 across the at least three replicates, of the nucleic acid sample to the one or more nucleic acid molecules of known identity comprising the array relative to a nucleic acid sample obtained from an animal which has not been subjected to the pain is indicative of the differential expression of the nucleotide sequence in the animal subjected to pain.

The invention still further provides a method for identifying a nucleotide sequence which is differentially regulated in an animal subjected to pain, comprising: hybridizing a nucleic acid sample corresponding to RNA obtained from an animal which has been subjected to pain to an array comprising a solid substrate and a plurality of nucleic acid members; wherein each nucleic acid member has a unique position and is stably associated with the solid substrate; and measuring the hybridization of the nucleic acid sample to the array, wherein a 1.4 fold difference in the hybridization of the nucleic acid sample to one or more nucleic acid members comprising the array relative to a nucleic acid sample obtained from an animal which has not been subjected to the pain is indicative of the differential expression of the nucleotide sequence in the animal subjected to pain.

In one embodiment, any of the preceding methods for identifying a nucleotide sequence which is differentially regulated in an animal subjected to pain may further comprise the step of verifying the differential expression of the nucleotide sequence by a molecular procedure selected from the group consisting of Northern analysis, in situ hybridization, and PCR.

The invention provides a method for identifying a nucleotide sequence which is differentially regulated in an animal subjected to pain, comprising: hybridizing a nucleic acid sample corresponding to RNA obtained from an animal which has been subjected to pain to an array comprising a solid substrate and a plurality of nucleic acid members; wherein each nucleic acid member has a unique position and is stably associated with the solid substrate; measuring the hybridization of the nucleic acid sample to the array, wherein a 1.4 fold difference in the hybridization of the nucleic acid sample to one or more nucleic acid members comprising the array relative to a nucleic acid sample obtained from an animal which has not been subjected to the pain is indicative of the differential expression of the nucleotide sequence in the animal subjected to pain; and verifying the differential expression of the nucleotide sequence by a molecular procedure selected from the group consisting of Northern analysis, in situ hybridization, and PCR.

In one embodiment, a 1.4 fold change in the hybridization of the nucleic acid sample to one or more nucleic acid members comprising the array relative to a nucleic acid sample obtained from an animal which has not been subjected to the pain is indicative of the differential expression of the nucleotide sequence following pain.

In a further embodiment, a 2 fold change in the hybridization of the nucleic acid sample to one or more nucleic acid members comprising the array relative to a nucleic acid sample obtained from an animal which has not been subjected to the pain is indicative of the differential expression of the nucleotide sequence following pain.

In one embodiment, the nucleic acid sample is labeled with a detectable label prior to the hybridization to the array.

In a further embodiment, the above methods for identifying a nucleic acid sequence which is differentially regulated in an animal subjected to pain further comprises the step of isolating the nucleic acid sample from the animal.

In one embodiment, nucleic acid sample is cRNA.

The present invention also provides an array comprising: a plurality of polynucleotide members, wherein each of the polynucleotide members is selected from Table 1, 2, 3, 4, or 5 and wherein at least one of the isolated polynucleotides is unique to Table 2, 3, 4, or 5; and a solid substrate, wherein each polynucleotide member has a unique position on the array and is stably associated with the solid substrate. Such an array will be referred to herein as a “pain specific array”.

The invention still further provides an array comprising: a plurality of polynucleotide members, wherein each of the polynucleotide members is selected from Table 1, 2, 3, 4, or 5, and wherein at least one of the isolated polynucleotides is unique to Table 2, 3, 4, or 5 and wherein the plurality of polynucleotide members are obtained from neuronal tissue obtained from at least two different species of animal; and a solid substrate, wherein each polynucleotide member obtained from each of the two different species has a unique position on the array and is stably associated with the solid substrate. Such an array will be referred to herein as a “pain specific array”.

The invention also comprises an array comprising: (a) a plurality of polynucleotide members, wherein each of said plurality of polynucleotides is selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 2 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 2 in the column designated “identifier”; and (b) a solid substrate, wherein each polynucleotide member has a unique position on said array and is stably associated with said solid substrate.

The present invention also provides an array comprising: a plurality of polynucleotide members, wherein each of the polynucleotide members is selected from Table 10 and/or 11; and a solid substrate, wherein each polynucleotide member has a unique position on the array and is stably associated with the solid substrate. Such an array will be referred to herein as a “pain specific array”.

The invention still further provides an array comprising: a plurality of polynucleotide members, wherein each of the polynucleotide members is selected from Table 10 and/or 11, wherein the plurality of polynucleotide members are obtained from neuronal tissue obtained from at least two different species of animal; and a solid substrate, wherein each polynucleotide member obtained from each of the two different species has a unique position on the array and is stably associated with the solid substrate. Such an array will be referred to herein as a “pain specific array”.

The invention also comprises an array comprising: (a) a plurality of polynucleotide members, wherein each of said plurality of polynucleotides is selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 10 and/or 11 in the column designated “identifier”; and (b) a solid substrate, wherein each polynucleotide member has a unique position on said array and is stably associated with said solid substrate.

In one embodiment, the plurality of polynucleotide members is differentially expressed by at least 1.2 fold across at least three replicate assays of expression in neuronal tissue of an animal subjected to pain with a p-value of less than 0.05 relative to an animal not subjected to the pain.

In one embodiment, the plurality of polynucleotide members is differentially expressed by at least 1.4 fold in the neurons of the animal subjected to pain relative to an animal not subjected to the pain.

In a further embodiment, the array comprises from 10 to 20,000 polynucleotide members.

In one embodiment, the array further comprises negative and positive control sequences and quality control sequences selected from the group consisting of cDNA sequences encoded by housekeeping genes, plant gene sequences, bacterial sequences, PCR products and vector sequences.

The invention further provides a method of identifying an agent that increases or decreases the expression of a polynucleotide sequence that is differentially expressed in neuronal tissue of a first animal which is subjected to pain comprising: administering the agent to the first animal; hybridizing nucleic acid isolated from one or more sensory neurons of the first and a second animal to a pain specific array; and measuring the hybridization of the nucleic acid isolated from the neuronal tissue of the first and second animal to the array; wherein an increase in hybridization of the nucleic acid from the first animal to one or more nucleic acid members of the array relative to hybridization of the nucleic acid from a second animal which is subjected to pain but to which is not administered the agent to one or more nucleic acid members of the array identifies the agent as increasing the expression of the polynucleotide sequence, and wherein a decrease in hybridization of the nucleic acid from the first animal to one or more nucleic acid members of the array relative to the hybridization of the nucleic acid from second animal to one or more nucleic acid members of the array identifies the agent as decreasing the expression of the polynucleotide sequence.

In one embodiment, the preceding method further comprises the step of verifying the increase or decrease in the hybridization by a molecular procedure selected from the group consisting of Northern analysis, in situ hybridization, and PCR.

In one embodiment, the nucleic acid sample isolated from the first and second animal is labeled with a detectable label prior to the hybridization to the array.

In a further embodiment, the nucleic acid sample isolated from the first animal is labeled with a different detectable label than the nucleic acid sample isolated from the second animal.

The invention also provides a method for identifying a compound which regulates the expression of a polynucleotide sequence which is differentially expressed in an animal subjected to pain, comprising: (a) providing a cell comprising and capable of expressing one or more of the polynucleotide selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 2 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 2 in the column designated “identifier”; (b) contacting said cell with a candidate compound; and (c) measuring the expression of said one or more of the polynucleotide specified supra, wherein if the expression of said differentially expressed polynucleotide sequence is increased in an animal which is subjected to pain, then said candidate modulator will be considered to regulate the expression of said polynucleotide if the expression of said polynucleotide is decreased by at least 10% in the presence of said candidate modulator, and wherein if the expression of said differentially expressed polynucleotide sequence is decreased in an animal subjected to pain, then said candidate modulator will be considered to regulate the expression of said polynucleotide if the expression of said polynucleotide is increased by at least 10% in the presence of said candidate modulator.

The invention also provides a method for identifying a compound which regulates the expression of a polynucleotide sequence which is differentially expressed in an animal subjected to pain, comprising: (a) providing a cell comprising and capable of expressing one or more of the polynucleotide selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 10 and/or 11 in the column designated “identifier”; (b) contacting said cell with a candidate compound; and (c) measuring the expression of said one or more of the polynucleotide specified supra, wherein if the expression of said differentially expressed polynucleotide sequence is increased in an animal which is subjected to pain, then said candidate modulator will be considered to regulate the expression of said polynucleotide if the expression of said polynucleotide is decreased by at least 10% in the presence of said candidate modulator, and wherein if the expression of said differentially expressed polynucleotide sequence is decreased in an animal subjected to pain, then said candidate modulator will be considered to regulate the expression of said polynucleotide if the expression of said polynucleotide is increased by at least 10% in the presence of said candidate modulator.

The invention also provides a method for identifying a compound which regulates the expression of a polynucleotide sequence which is differentially expressed in an animal subjected to pain, comprising: providing a cell comprising and capable of expressing one or more of the polynucleotide sequences shown in Tables 1, 2, 3, 4, or 5; contacting the cell with a candidate compound; and measuring the expression of the one or more of the polynucleotide sequences shown in Tables 1, 2, 3, 4, or 5, wherein an increase or decrease in the expression of the one or more of the polynucleotide sequences shown in Table 1, 2, 3, 4, or 5 of at least 10% is indicative of regulation of the differentially expressed polynucleotide sequence.

The invention still further provides a method for identifying a compound which regulates the activity of one or more of the polypeptides shown in Table 1, 2, 3, 4, or 5, or the activity of a polypeptide encoded by a polynucleotide sequence indicated in Table 1, 2, 3, 4, or 5 comprising: providing a cell comprising the one or more polypeptides; contacting the cell with a candidate compound; and measuring the activity of the one or more polypeptides, wherein an increase or decrease of the activity of the one or more polypeptides of at least 10% relative to the activity of the one or more polypeptides in the cell, wherein the cell is not contacted with the candidate compound, identifies the candidate compound as a compound which regulates the activity of the one or more polypeptides.

The invention also provides a method for identifying a compound which regulates the expression of a polynucleotide sequence which is differentially expressed in an animal subjected to pain, comprising: providing a cell comprising and capable of expressing one or more of the polynucleotide sequences shown in Tables 10 and/or 11; contacting the cell with a candidate compound; and measuring the expression of the one or more of the polynucleotide sequences shown in Tables 10 and/or 11, wherein an increase or decrease in the expression of the one or more of the polynucleotide sequences shown in Table 10 and/or 11 of at least 10% is indicative of regulation of the differentially expressed polynucleotide sequence.

The invention still further provides a method for identifying a compound which regulates the activity of one or more of the polypeptides shown in Table 10 and/or 11, or the activity of a polypeptide encoded by a polynucleotide sequence indicated in Table 10 and/or 111 comprising: providing a cell comprising the one or more polypeptides; contacting the cell with a candidate compound; and measuring the activity of the one or more polypeptides, wherein an increase or decrease of the activity of the one or more polypeptides of at least 10% relative to the activity of the one or more polypeptides in the cell, wherein the cell is not contacted with the candidate compound, identifies the candidate compound as a compound which regulates the activity of the one or more polypeptides.

In one embodiment, the candidate compound is selected from the group consisting of small molecule, protein, RNAi, and antisense.

In a further embodiment, the candidate compound is an antibody which binds to the polypeptide.

The invention also provides a method for producing a pharmaceutical formulation comprising: providing a cell comprising the one or more polypeptides; selecting a compound which regulates the activity of the one or more polypeptides; and mixing the compound with a carrier.

In one embodiment, the step of selecting comprises the steps of contacting the cell with a candidate compound; and measuring the activity of the one or more polypeptides, wherein an increase or decrease of the activity of the one or more polypeptides of at least 10% relative to the activity of the one or more polypeptides in the cell, wherein the cell is not contacted with the candidate compound, identifies the candidate compound as a compound which regulates the activity of the one or more polypeptides.

The invention also provides a method for producing a pharmaceutical formulation comprising: (a) providing a cell comprising said one or more polypeptides encoded by a polynucleotide selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 2 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 2 in the column designated “identifier”; (b) selecting a compound which regulates the activity of said one or more polypeptides; and (c) mixing said compound with a carrier.

The invention also provides a method for producing a pharmaceutical formulation comprising: (a) providing a cell comprising said one or more polypeptides encoded by a polynucleotide selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 10 and/or 11 in the column designated “identifier”; (b) selecting a compound which regulates the activity of said one or more polypeptides; and (c) mixing said compound with a carrier.

In one embodiment, the step of selecting comprises the steps of contacting said cell with a candidate compound; and measuring the activity of said one or more polypeptides, wherein an increase or decrease of the activity of said one or more polypeptides of at least 10% relative to the activity of said one or more polypeptides in said cell, wherein the cell is not contacted with the candidate compound, identifies said candidate compound as a compound which regulates the activity of said one or more polypeptides

The invention also provides a method for identifying a compound which regulates the activity, in an animal, of one or more of the polypeptides shown in Table 1, 2, 3, 4, or 5, or a polypeptide encoded by one or more polynucleotide sequence indicated in Table 1, 2, 3, 4, or 5 comprising: administering a candidate compound to an animal comprising the one or more polypeptides; and measuring the activity of the one or more polypeptides wherein an increase or decrease of the activity of the polypeptide of at least 10% relative to the activity of the one or more polypeptides in an animal to which the candidate compound is not administered, identifies the candidate compound as a compound which regulates the activity of the one or more polypeptides.

The invention also provides a method for identifying a compound which regulates the activity, in an animal, of one or more of the polypeptides shown in Table 10 and/or 11, or a polypeptide encoded by one or more polynucleotide sequence indicated in Table 10 and/or 11 comprising: administering a candidate compound to an animal comprising the one or more polypeptides; and measuring the activity of the one or more polypeptides wherein an increase or decrease of the activity of the polypeptide of at least 10% relative to the activity of the one or more polypeptides in an animal to which the candidate compound is not administered, identifies the candidate compound as a compound which regulates the activity of the one or more polypeptides.

Preferably, the candidate compound is selected from the group consisting of small molecule, protein, RNAi, and antisense.

In one embodiment, the candidate compound is an antibody which binds to the polypeptide.

The invention still further provides a method for identifying a small molecule which regulates the activity of one or more of the polypeptides indicated in Table 1, 2, 3, 4, or 5, or a polypeptide encoded by one or more polynucleotides indicated in Table 1, 2, 3, 4, or 5 comprising: providing a cell comprising the one or more polypeptides; generating a small molecule library; providing a candidate small molecule, selected from the library; contacting the cell with the candidate small molecule; and measuring the activity of the one or more polypeptides, wherein an increase or decrease of the activity of the one or more polypeptides of at least 10% relative to the activity of the one or more polypeptides in the cell, wherein the cell is not contacted with the candidate small molecule, identifies the candidate small molecule as a small molecule which regulates the activity of the one or more polypeptides.

The invention still further provides a method for identifying a small molecule which regulates the activity of one or more of the polypeptides indicated in Table 10 and/or 11, or a polypeptide encoded by one or more polynucleotides indicated in Table 10 and/or 11 comprising: providing a cell comprising the one or more polypeptides; generating a small molecule library; providing a candidate small molecule, selected from the library; contacting the cell with the candidate small molecule; and measuring the activity of the one or more polypeptides, wherein an increase or decrease of the activity of the one or more polypeptides of at least 10% relative to the activity of the one or more polypeptides in the cell, wherein the cell is not contacted with the candidate small molecule, identifies the candidate small molecule as a small molecule which regulates the activity of the one or more polypeptides.

Preferably, the small molecule library comprises components selected from the group consisting of heterocyclics, aromatics, alicyclics, aliphatics, steroids, antibiotics, enzyme inhibitors, ligands, hormones, alkaloids, opioids, terpenes, porphyrins, toxins, and catalysts, and combinations thereof.

The invention also relates to a method for identifying a small molecule which regulates the activity of one or more of the polypeptides indicated in Table 2, comprising: (a) providing a cell comprising said one or more polypeptides encoded by a polynucleotide selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 2 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 2 in the column designated “identifier”; (b) generating a small molecule library; (c) providing a candidate small molecule, selected from said library; (d) contacting said cell with said candidate small molecule; and (e) measuring the activity of said one or more polypeptides, wherein an increase or decrease of the activity of said one or more polypeptides of at least 10% relative to the activity of said one or more polypeptides in said cell, wherein the cell is not contacted with the candidate small molecule, identifies said candidate small molecule as a small molecule which regulates the activity of said one or more polypeptides.

The invention also relates to a method for identifying a small molecule which regulates the activity of one or more of the polypeptides indicated in Table 10 and/or 11, comprising: (a) providing a cell comprising said one or more polypeptides encoded by a polynucleotide selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 10 and/or 11 in the column designated “identifier”; (b) generating a small molecule library; (c) providing a candidate small molecule, selected from said library; (d) contacting said cell with said candidate small molecule; and (e) measuring the activity of said one or more polypeptides, wherein an increase or decrease of the activity of said one or more polypeptides of at least 10% relative to the activity of said one or more polypeptides in said cell, wherein the cell is not contacted with the candidate small molecule, identifies said candidate small molecule as a small molecule which regulates the activity of said one or more polypeptides.

The invention further relates to a method for identifying a compound useful in the treatment of pain, comprising: providing a host cell comprising a vector comprising one or more of the polynucleotides identified in Table 1, 2, 3, 4, or 5; maintaining the host cell under conditions which permit the expression of the one or more polynucleotides; selecting a compound which regulates the activity of a polypeptide encoded by the one or more polynucleotides; administering the compound to an animal subjected to pain; and measuring the level of pain in the animal, wherein a decrease in the level of pain in the animal of at least 10%, identifies the compound as being useful for treating pain.

The invention further relates to a method for identifying a compound useful in the treatment of pain, comprising: providing a host cell comprising a vector comprising one or more of the polynucleotides identified in Table 10 and/or 11; maintaining the host cell under conditions which permit the expression of the one or more polynucleotides; selecting a compound which regulates the activity of a polypeptide encoded by the one or more polynucleotides; administering the compound to an animal subjected to pain; and measuring the level of pain in the animal, wherein a decrease in the level of pain in the animal of at least 10%, identifies the compound as being useful for treating pain.

In one embodiment, the step of selecting includes the steps of contacting the cell with a candidate compound; and measuring the activity of the polypeptide encoded by the one or more polynucleotides, wherein an increase or decrease of the activity of the polypeptide of at least 10% relative to the activity of the polypeptide in the cell, wherein the cell is not contacted with the candidate compound, identifies the candidate compound as a compound which regulates the activity of the polypeptide.

The invention further provides a method for identifying a compound useful in the treatment of pain, comprising: (a) providing a host cell comprising a vector comprising one or more of the polynucleotides selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 2 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 2 in the column designated “identifier”; (b) maintaining said host cell under conditions which permit the expression of said one or more polynucleotides; (c) selecting a compound which regulates the activity of a polypeptide encoded by said one or more polynucleotides; (d) administering said compound to an animal subjected to pain; and (e) measuring the level of pain in said animal, wherein a decrease in the level of pain in said animal of at least 10%, identifies said compound as being useful for treating pain.

The invention further provides a method for identifying a compound useful in the treatment of pain, comprising: (a) providing a host cell comprising a vector comprising one or more of the polynucleotides selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (i) to (ii) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (i) to (iii) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (i) to (iv) and encodes a polypeptide exhibiting the biological function as specified for the respective sequence in Table 10 and/or 11 in the column designated “identifier”; (b) maintaining said host cell under conditions which permit the expression of said one or more polynucleotides; (c) selecting a compound which regulates the activity of a polypeptide encoded by said one or more polynucleotides; (d) administering said compound to an animal subjected to pain; and (e) measuring the level of pain in said animal, wherein a decrease in the level of pain in said animal of at least 10%, identifies said compound as being useful for treating pain.

In one embodiment, the step of selecting includes the steps of contacting said cell with a candidate compound; and measuring the activity of the polypeptide encoded by said one or more polynucleotides, wherein an increase or decrease of the activity of said polypeptide of at least 10% relative to the activity of said polypeptide in said cell, wherein the cell is not contacted with the candidate compound, identifies said candidate compound as a compound which regulates the activity of said polypeptide.

The invention also provides a method of treating pain in an animal comprising administering to the animal an antisense polynucleotide capable of inhibiting the expression of one or more of the polynucleotide sequences indicated in Table 1, 2, 3, 4, or 5.

The invention further provides a method of treating pain in an animal comprising administering to the animal a double stranded RNA molecule wherein one of the strands of the double stranded RNA molecule is identical to a portion of an mRNA transcript obtained from one or more of the polynucleotide sequences indicated in Table 1, 2, 3, 4, or 5.

The invention still further provides a method of treating pain in an animal in need thereof, comprising: administering to the animal a therapeutically effective amount of an agent which modulates the activity of one or more of the polypeptides indicated in Table 1, 2, 3, 4, or 5, or a polypeptide encoded by one or more of the polynucleotides indicated in Table 1, 2, 3, 4, or 5.

The invention also provides a method of treating pain in an animal in need thereof, comprising: administering a therapeutically effective amount of an antibody which binds to one or more of the polypeptides indicated in Table 1, 2, 3, 4, or 5, or a polypeptide encoded by one or more of the polynucleotides indicated in Table 1, 2, 3, 4, or 5.

The invention still further provides a method of treating pain in an animal in need thereof, comprising: administering a therapeutically effective amount of one or more of the polypeptides indicated in Table 1, 2, 3, 4, or 5, or a polypeptide encoded by one or more of the polynucleotides indicated in Table 1, 2, 3, 4, or 5.

The invention further relates to a method of detecting pain in an animal suspected of having pain comprising: measuring the amount of one or more of the polynucleotide sequences of Table 10 or 11 in an animal suspected of having pain, wherein if said amount of the one or more polynucleotides is increased or decreased by at least 1.2 fold across at least three replicate screens and is statistically significant at P<0.05 in the animal compared to the amount of the same one or more polynucletoide sequences of Table 10 or 11 in an animal not suspected of having pain, then pain is detected in the animal suspected of having pain.

In one embodiment, the amount of said one or more polynucleotide sequences is measured in one or more sensory neurons.

In a further embodiment, the sensory neurons are selected from the group consisting of dorsal horn neurons and dorsal root ganglion neurons.

In one embodiment the polynucleotide is measured in a “patient sample” wherein a “patient sample” as used herein refers to blood, plasma, serum, or cerebro-spinal fluid.

In one embodiment, the polynucletoide is differentially expressed by 1.4 fold.

The invention further relates to a method of detecting pain in an animal suspected of having pain comprising: measuring the amount of one or more of the polynucleotide sequences of Table 1, 2, 3, 4, or 5 in an animal suspected of having pain, wherein if said amount of the one or more polynucleotides is increased or decreased by at least 1.2 fold across at least three replicate screens and is statistically significant at P<0.05 in the animal compared to the amount of the same one or more polynucletoide sequences of Table 1, 2, 3, 4, or 5 in an animal not suspected of having pain, then pain is detected in the animal suspected of having pain.

In one embodiment, the amount of said one or more polynucleotide sequences is measured in one or more sensory neurons.

In a further embodiment, the sensory neurons are selected from the group consisting of dorsal horn neurons and dorsal root ganglion neurons.

In one embodiment the polynucleotide is measured in a “patient sample” wherein a “patient sample” as used herein refers to blood, plasma, serum, or cerebro-spinal fluid.

In one embodiment, the polynucletoide is differentially expressed by 1.4 fold.

A method of detecting pain in an animal suspected of having pain comprising: measuring the amount of one or more of the polypeptide sequences of Table 10 or 11 in the animal suspected of having pain, wherein if the amount of said one or more polypeptides is increased or decreased by at least 1.2 fold across at least three replicate screens and is statistically significant at P<0.05 in the animal compared to the amount of the same one or more polypeptide sequences of Table 10 or 11 in an animal not suspected of having pain, then pain is detected in the animal suspected of having pain.

In one embodiment, the amount of said one or more polynucleotide sequences is measured in one or more sensory neurons.

In a further embodiment, the sensory neurons are selected from the group consisting of dorsal horn neurons and dorsal root ganglion neurons.

In one embodiment the polypeptide is measured in a “patient sample” wherein a “patient sample” as used herein refers to blood, plasma, serum, or cerebro-spinal fluid.

In one embodiment, the polypeptide is differentially expressed by 1.4 fold.

The invention still further provides a method of detecting pain in an animal suspected of having pain comprising: measuring the amount of one or more of the polypeptide sequences of Table 1, 2, 3, 4, or 5 in the animal suspected of having pain, wherein if the amount of said one or more polypeptides is increased or decreased by at least 1.2 fold across at least three replicate screens and is statistically significant at P<0.05 in the animal compared to the amount of the same one or more polypeptide sequences of Table 1, 2, 3, 4, or 5 in an animal not suspected of having pain, then pain is detected in the animal suspected of having pain.

In one embodiment, the amount of said one or more polypeptide sequences is measured in one or more sensory neurons.

In a further embodiment, the sensory neurons are selected from the group consisting of dorsal horn neurons and dorsal root ganglion neurons.

In one embodiment the polypeptide is measured in a “patient sample” wherein a “patient sample” as used herein refers to blood, plasma, serum, or cerebro-spinal fluid.

In one embodiment, the polypeptide is differentially expressed by 1.4 fold.

The invention also provides a pharmaceutical formulation comprising one or more polypeptides indicated in Table 1, 2, 3, 4, or 5, or a polypeptide encoded by one or more of the polynucleotides indicated in Table 1, 2, 3, 4, or 5, and a carrier.

The invention also provides a pharmaceutical formulation comprising one or more antibodies which bind to one or more of the polypeptides indicated in Table 1, 2, 3, 4, or 5, or a polypeptide encoded by one or more of the polynucleotides indicated in Table 1, 2, 3, 4, or 5, and a carrier.

The invention also provides a method of treating pain in an animal comprising administering to the animal an antisense polynucleotide capable of inhibiting the expression of one or more of the polynucleotide sequences indicated in Table 10 and/or 11.

The invention further provides a method of treating pain in an animal comprising administering to the animal a double stranded RNA molecule wherein one of the strands of the double stranded RNA molecule is identical to a portion of an mRNA transcript obtained from one or more of the polynucleotide sequences indicated in Table 10 and/or 11.

The invention still further provides a method of treating pain in an animal in need thereof, comprising: administering to the animal a therapeutically effective amount of an agent which modulates the activity of one or more of the polypeptides indicated in Table 10 and/or 11, or a polypeptide encoded by one or more of the polynucleotides indicated in Table 10 and/or 11.

The invention also provides a method of treating pain in an animal in need thereof, comprising: administering a therapeutically effective amount of an antibody which binds to one or more of the polypeptides indicated in Table 10 and/or 11, or a polypeptide encoded by one or more of the polynucleotides indicated in Table 10 and/or 11.

The invention still further provides a method of treating pain in an animal in need thereof, comprising: administering a therapeutically effective amount of one or more of the polypeptides indicated in Table 10 and/or 11, or a polypeptide encoded by one or more of the polynucleotides indicated in Table 10 and/or 11.

The invention also provides a pharmaceutical formulation comprising one or more polypeptides indicated in Table 10 and/or 11, or a polypeptide encoded by one or more of the polynucleotides indicated in Table 10 and/or 11, and a carrier.

The invention also provides a pharmaceutical formulation comprising one or more antibodies which bind to one or more of the polypeptides indicated in Table 10 and/or 11, or a polypeptide encoded by one or more of the polynucleotides indicated in Table 10 and/or 11, and a carrier.

The invention further relates to the use of: (a) a polynucleotide selected from the group consisting of: (i) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (ii) a polynucleotide encoding an amino acid sequence selected from the group consisting of: (1) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (2) the amino acid specified in Table 2 in the columns designated “rat protein” and “human protein”; (iii) a polynucleotide which hybridizes under high stringency conditions to a polynucleotide specified in (a) to (b) and encodes a polypeptide exhibiting the biological function as specified for the respective sequence in Table 2 in the column designated “identifier”; (iv) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; (v) a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”; (vi) a polypeptide encoded by any of the polynucleotides specified in (i) to (v); in the preparation of a medicament for the treatment of pain in an animal.

The invention further relates to the use of: (a) a polynucleotide selected from the group consisting of: (i) a polynucleotide comprising any of the polynucleotides specified in Table 10 and/or 11 in the columns designated “rat gene” and “human gene”; (ii) a polynucleotide encoding an amino acid sequence selected from the group consisting of: (1) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (2) the amino acid specified in Table 10 and/or 11 in the columns designated “rat protein” and “human protein”; (iii) a polynucleotide which hybridizes under high stringency conditions to a polynucleotide specified in (a) to (b) and encodes a polypeptide exhibiting the biological function as specified for the respective sequence in Table 10 and/or 11 in the column designated “identifier”; (iv) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 10 and/or 11 in the column designated “identifier”; (v) a polynucleotide which represents a fragment, derivative or allelic variation of a nucleic acid sequence specified in (a) to (d) and encodes a polypeptide exhibiting the biological function as specified for the respective sequence in Table 10 and/or 11 in the column designated “identifier”; (vi) a polypeptide encoded by any of the polynucleotides specified in (i) to (v); in the preparation of a medicament for the treatment of pain in an animal.

The present invention still further relates to the use of a compound which can modulate the activity of a polypeptide which is encoded by a polynucleotide selected from the group consisting of: (a) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (b) a polynucleotide encoding an amino acid sequence selected from the group consisting of: (i) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (ii) the amino acid specified in Table 2 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 2 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”; in the preparation of a medicament for the treatment of pain in an animal.

The present invention provides a pharmaceutical formulation comprising one or more polypeptides encoded by a polynucleotide selected from the group consisting of: (a) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (b) a polynucleotide encoding an amino acid sequence selected from the group consisting of: (i) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (ii) the amino acid specified in Table 2 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 2 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”; and a carrier.

The invention still further provides a pharmaceutical formulation comprising one or more antibodies which bind to one or more of the polypeptides encoded by a polynucleotide selected from the group consisting of: (a) a polynucleotide comprising any of the polynucleotides specified in Table 1-2 in the columns designated “rat gene” and “human gene”, and wherein at least one of said two or more isolated polynucleotides is unique to Table 2 in the columns designated “rat gene” and “human gene”; (b) a polynucleotide encoding an amino acid sequence selected from the group consisting of: (i) amino acid sequences which are homologue to any of the amino acid specified in Table 2 in the columns designated “rat protein” and “human protein” by at least the homology as specified for the respective sequence in Table 2 in the column designated “% homology” and encodes a polypeptide exhibiting the biological function as specified for the respective sequence in Table 2 in the column designated “identifier”; (ii) the amino acid specified in Table 2 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 2 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 2 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 2 in the column designated “identifier”; and a carrier.

The present invention still further relates to the use of a compound which can modulate the activity of a polypeptide which is encoded by a polynucleotide selected from the group consisting of: (a) a polynucleotide comprising any of the polynucleotides specified in Table 10 and/or 11 in the columns designated “rat gene” and “human gene”; (b) a polynucleotide encoding an amino acid sequence selected from the group consisting of: (i) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (ii) the amino acid specified in Table 10 and/or 11 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 10 and/or 11 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 10 and/or 11 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 10 and/or 11 in the column designated “identifier”; in the preparation of a medicament for the treatment of pain in an animal.

The present invention provides a pharmaceutical formulation comprising one or more polypeptides encoded by a polynucleotide selected from the group consisting of: (a) a polynucleotide comprising any of the polynucleotides specified in Table 10 and/or 111 in the columns designated “rat gene” and “human gene”; (b) a polynucleotide encoding an amino acid sequence selected from the group consisting of: (i) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (ii) the amino acid specified in Table 10 and/or 11 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 10 and/or 11 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 10 and/or 11 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 10 and/or 11 in the column designated “identifier”; and a carrier.

The invention still further provides a pharmaceutical formulation comprising one or more antibodies which bind to one or more of the polypeptides encoded by a polynucleotide selected from the group consisting of: (a) a polynucleotide comprising any of the polynucleotides specified in Table 10 and/or 11 in the columns designated “rat gene” and “human gene”; (b) a polynucleotide encoding an amino acid sequence selected from the group consisting of: (i) amino acid sequences which are homologue to any of the amino acid specified in Table 10 and/or 11 in the columns designated “rat protein” and “human protein” by at least the homology as specified for the respective sequence in Table 10 and/or 11 in the column designated “% homology” and encodes a polypeptide exhibiting the biological function as specified for the respective sequence in Table 10 and/or 11 in the column designated “identifier”; (ii) the amino acid specified in Table 10 and/or 11 in the columns designated “rat protein” and “human protein”; (c) a polynucleotide which hybridizes under high stringency conditions to a polynucleotide specified in (a) to (b) and encodes a polypeptide exhibiting the biological function as specified for the respective sequence in Table 10 and/or 11 in the column designated “identifier”; (d) a polynucleotide the nucleic acid sequence or which deviates from the nucleic acid sequences specified in (a) to (c) due to the degeneration of the genetic code and encodes a polypeptide exhibiting the biological function as specified for the respective sequence in Table 10 and/or 11 in the column designated “identifier”; (e) a polynucleotide which represents a fragment, derivative or allelic variation of a nucleic acid sequence specified in (a) to (d) and encodes a polypeptide exhibiting the biological function as specified for the respective sequence in Table 10 and/or 11 in the column designated “identifier”; and a carrier.

According to the invention, a sequence differentially expressed under pain conditions must be differentially expressed in the neurons of an animal subjected to nerve injury, or inflammatory pain, thus differential expression in an animal subjected to nerve injury pain is determined, according to the invention, in one or all of the following nerve injury pain models. A sequence which is differentially expressed according to the invention is a sequence which is differentially expressed in (1) an axotomy pain model, (2) a spared nerve injury pain model, (3) chronic constriction pain model, (4) spinal segmental nerve lesion pain model, or (5) an inflammation pain model, or may be differentially expressed in all five pain models, or in a combination of two or more models.

As used herein differential expression of a sequence in nerve tissue is determined in either a “nerve injury pain model” or a “inflammation pain model”, or both. There are four alternate nerve injury pain models by which differential expression can be determined according to the invention: axotomy, spared nerve injury (SNI), spinal segmental nerve lesion, and chronic constriction.

As used herein, an “axotomy pain model” refers to a situation in which one or a plurality of peripheral nerve fibers is severed, either by traumatic injury or experimental or surgical manipulation. An “axotomy pain model” may further refer to an experimental model in which all of the axons of a given population of nerve cells are completely severed. For example, an “axotomy pain model” useful in the present invention may be a model in which all of the axons that comprise the sciatic nerve are surgically cut. All of the nerve cells in the dorsal root ganglion which gave rise to the axons of the sciatic nerve are thus said to be “axotomized”.

As used herein, a “spared nerve injury pain model” refers to a situation in which one of the terminal branches of the sciatic nerve is spared from axotomy (Decosterd and Woolf, 2000 Pain 87: 149). The SNI procedure comprises an axotomy and ligation of the tibial and common peroneal nerves leaving the sural nerve intact.

As used herein, a “spinal segmental nerve lesion” and “chronic constriction” refer to two types of “neuropathic pain models” useful in the present invention. Both models are well known to those of skill in the art (See, for example Kim and Chung, 1992 Pain 50: 355; and Bennett, 1993 Muscle Nerve 16: 1040 for a description of the “segmental nerve lesion” and “chronic constriction” respectively). A “segmental nerve lesion” and/or “chronic constriction” neuropathic pain model may be evaluated for the presence of “pain” using any of the behavioral, electrophysiological, and/or neurochemical criteria described below.

As used herein, an “inflammatory pain model” refers to a situation in which an animal is subjected to pain, as defined herein, by the induction of peripheral tissue inflammation (Stein et al., (1988) Pharmacol Biochem Behav 31: 445-451; Woolf et al., (1994) Neurosci. 62, 327-331). The inflammation can be produced by injection of an irritant such as complete Freunds adjuvant (CFA), carrageenan, turpentine, croton oil, and the like into the skin, subcutaneously, into a muscle, into a joint, or into a visceral organ. In addition, an “inflammatory pain model” can be produced by the administration of cytokines or inflammatory mediators such as lippopolysoccharide (LPS), or nerve growth factor (NGF) which can mimic the effects of inflammation. An “inflammatory pain model” can be evaluated for the presence of “pain” using behavioral, electrophysiological, and/or neurochemical criteria as described below.

A polynucleotide is thus differentially expressed herein if it is differentially expressed in any or all of the axotomy, SNI, chronic constriction, segmental nerve lesion and inflammatory pain models.

As used herein, “nerve tissue” refers to animal tissue comprising nerve cells, the neuropil, glia, neural inflammatory cells, and endothelial cells in contact with “nerve tissue”. “Nerve cells” may be any type of nerve cell known to those of skill in the art including, but not limited to motor neurons, sensory neurons, enteric neurons, sympathetic neurons, parasympathetic neurons, association neurons, and central nervous system neurons. “Glial cells” useful in the present invention include, but are not limited to astrocytes, Schwan cells, and oligodendrocytes. “Neural inflammatory cells” useful in the present invention include, but are not limited to microglia. Preferably, “nerve tissue” as used herein refers to nerve cells obtained from the dorsal root ganglion, or dorsal horn of the spinal cord.

As used herein, “sensory neuron” refers to any sensory neuron in an animal. A “sensory neuron” can be a peripheral sensory neuron, central sensory neuron, or enteric sensory neuron. A “sensory neuron” includes all parts of a neuron including, but not limited to the cell body, axon, and dendrite(s). A “sensory neuron” refers to a neuron which receives and transmits information (encoded by a combination of action potentials, neurotransmitters and neuropeptides) relating to sensory input, including, but not limited to pain, heat, touch, cold, pressure, vibration, etc. Examples of “sensory neurons” include, but are not limited to dorsal root ganglion neurons, dorsal horn neurons of the spinal cord, autonomic neurons, trigeminal ganglion neurons, and the like.

As used herein, “animal” refers to a organism classified within the phylogenetic kingdom Animalia. As used herein, an “animal” also refers to a mammal. Animals, useful in the present invention, include, but are not limited to mammals, marsupials, mice, dogs, cats, cows, humans, deer, horses, sheep, livestock, and the like.

As used herein, “subjected” refers to a state of being in which an animal is experiencing pain, wherein whether or not the animal is experiencing pain is determined using the behavioral, electrophysiological, and/or neurochemical criteria described above. As used herein, “subjected” does not refer to the past experience of pain only, but can also include the present experience of pain.

As used herein, “polynucleotide” refers to a polymeric form of nucleotides of 2 up to 1,000 bases in length, or even more, either ribonucleotides or deoxyribonucleotides or a modified form of either type of nucleotide. The term includes single and double stranded forms of DNA. The term is synonymous with “oligonucleotide”. Polynucleotides of the invention include those indicated by accession number in Tables 1, 2, 3, 4, or 5, or a portion thereof.

As used herein, “polypeptide” refers to any kind of polypeptide such as peptides, human proteins, fragments of human proteins, proteins or fragments of proteins from non-human sources, engineered versions proteins or fragments of proteins, enzymes, antigens, drugs, molecules involved in cell signaling, such as receptor molecules, antibodies, including polypeptides of the immunoglobulin superfamily, such as antibody polypeptides or T-cell receptor polypeptides. Preferably, a “polypeptide” useful according to the invention is indicated by accession number in Tables 1, 2, 3, 4, or 5. Also included, are a fragment, domain, or epitope of one or more of the polypeptides indicated in Tables 2, 3, 4, or 5 provided that the fragment, domain, or epitope maintains the same function as the protein indicated in Table 2, 3, 4, or 5, wherein the function of the polypeptide is known to those of skill in the art. Also included, are a fragment, domain, or epitope of one or more of the polypeptides indicated in Tables 2 or 3 provided that the fragment, domain, or epitope maintains the same function as the protein indicated in Table 2 or 3, under the column heading “identifier”, “description” or “protein type”

As used herein, the term “vector” refers to a nucleic acid molecule capable of transporting another nucleic acid to which it has been linked. One type of vector is a “plasmid”, which refers to a circular double stranded nucleic acid loop into which additional nucleic acid segments can be ligated. Another type of vector is a “viral vector”, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant nucleic acid techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “hybridizing” or “hybridization” refers to the hydrogen binding with a complementary nucleic acid, via an interaction between for example, a target nucleic acid sequence and a nucleic acid member in an array.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of two nucleic acids, for example a nucleic acid member to a target nucleic acid sequence. These factors include nucleic acid member length, nucleotide sequence and/or composition, hybridization temperature, buffer composition and potential for steric hindrance in the region to which the nucleic acid member is required to hybridize.

A positive correlation exists between the nucleic acid member length and both the efficiency and accuracy with which a nucleic acid member will anneal to a target sequence. In particular, longer sequences have a higher melting temperature (TM) than do shorter ones, and are less likely to be repeated within a given target sequence, thereby minimizing promiscuous hybridization. Hybridization temperature varies inversely with nucleic acid member annealing efficiency, as does the concentration of organic solvents, e.g., formamide, that might be included in a hybridization mixture, while increases in salt concentration facilitate binding. Under stringent annealing conditions, longer nucleic acids, hybridize more efficiently than do shorter ones, which are sufficient under more permissive conditions. As herein used, the term “standard stringent conditions” means hybridization will occur only if there is at least 95% and preferably at least 97% identity between the sequences, wherein the region of identity comprises at least 10 nucleotides. In one embodiment, the sequences hybridize under stringent conditions following incubation of the sequences overnight at 42° C., followed by stringent washes (0.2×SSC at 65° C.). As several factors affect the stringency of hybridization, the combination of parameters is more important than the absolute measure of a single factor.

As defined herein, an “array” refers a plurality of unique nucleic acids attached to one surface of a solid support at a density exceeding 20 different nucleic acids/cm² wherein each of the nucleic acids is attached to the surface of the solid support in a non-identical preselected region. In one embodiment, the nucleic acid attached to the surface of the solid support is DNA. In a preferred embodiment, the nucleic acid attached to the surface of the solid support is cDNA. In another preferred embodiment, the nucleic acid attached to the surface of the solid support is cDNA synthesized by polymerase chain reaction (PCR). Preferably, a nucleic acid comprising an array, according to the invention, is at least 20 nucleotides in length. Preferably, a nucleic acid comprising an array is less than 6,000 nucleotides in length. More preferably, a nucleic acid comprising an array is less than 500 nucleotides in length. In one embodiment, the array comprises at least 500 different nucleic acids attached to one surface of the solid support. In another embodiment, the array comprises at least 10 different nucleic acids attached to one surface of the solid support. In yet another embodiment, the array comprises at least 10,000 different nucleic acids attached to one surface of the solid support. The term “nucleic acid”, as used herein, is interchangeable with the term “polynucleotide”.

As used herein, “plurality” refers to more than two. Plurality, according to the invention, can be 3 or more, 100 or more, or 1000 or more.

As used herein, “attaching” or “spotting” refers to a process of depositing a nucleic acid onto a solid substrate to form a nucleic acid array such that the nucleic acid is irreversibly bound to the solid substrate via covalent bonds, hydrogen bonds or ionic interactions.

As used herein, “stably associated” refers to a nucleic acid that is irreversibly bound to a solid substrate to form an array via covalent bonds, hydrogen bonds or ionic interactions such that the nucleic acid retains its unique preselected position relative to all other nucleic acids that are stably associated with an array, or to all other preselected regions on the solid substrate under conditions wherein an array is analyzed (i.e., hybridization and scanning).

As used herein, “solid substrate” or “solid support” refers to a material having a rigid or semi-rigid surface. The terms “substrate” and “support” are used interchangeable herein with the terms “solid substrate” and “solid support”. The solid support may be biological, non-biological, organic, inorganic, or a combination of any of these, existing as particles, strands, precipitates, gels, sheets, tubing, spheres, containers, capillaries, pads, slices, films, plates, slides, etc. Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent.

As used herein, “preselected region”, “predefined region”, or “unique position” refers to a localized area on a substrate which is, was, or is intended to be used for the deposit of a nucleic acid and is otherwise referred to herein in the alternative as a “selected region” or simply a “region.” The preselected region may have any convenient shape, e.g., circular, rectangular, elliptical, wedge-shaped, etc. In some embodiments, a preselected region is smaller than about 1 cm², more preferably less than 1 mm², still more preferably less than 0.5 mm², and in some embodiments about 0.125 to 0.5 mm².

As used herein, “unique to Table X”, where “X” is one or more of 2, 3, 4, or 5, refers to a polynucleotide or polypeptide sequence which is indicated in Table X, but is not indicated in Table 1.

As used herein, the term “level of expression” refers to the measurable expression level of a given nucleic acid. The level of expression of a nucleic acid is determined by methods well known in the art. The term “differentially expressed” or “differential expression” refers to an increase or decrease in the measurable expression level of a given nucleic acid. As used herein, “differentially expressed” or “differential expression” means the difference in the level of expression of a nucleic acid is at least 1.4-fold or more in two samples used for comparison, both of which are compared to the same normal standard sample. “Differentially expressed” or “differential expression” according to the invention also means a 1.4-fold, or more, up to and including 2-fold, 5-fold, 10-fold, 20-fold, 50-fold or more difference in the level of expression of a nucleic acid in two samples used for comparison. A nucleic acid is also said to be “differentially expressed” in two samples if one of the two samples contains no detectable expression of a given nucleic acid, provided that the detectably expressed nucleic acid is expressed at +/−at least 1.4 fold. Differential expression of a nucleic acid sequence is “inhibited” the difference in the level of expression of the nucleic acid in two or more samples used for comparison is altered such that it is no longer at least a 1.4 fold difference. Absolute quantification of the level of expression of a nucleic acid may be accomplished by including a known concentration(s) of one or more control nucleic acid species, generating a standard curve based on the amount of the control nucleic acid and extrapolating the expression level of the “unknown” nucleic acid species from the hybridization intensities of the unknown with respect to the standard curve.

Alternatively, “differential expression”, according to the invention, refers to a 1.2 fold increase or decrease in the level of expression of a nucleic acid in an animal subjected to pain compared to the level of expression in an animal not subjected to the same pain, combined with a statistical significance of p<0.05 in at least three replicate assays of gene expression. Calculation of a statistically significant 1.2 fold threshold in the increase or decrease in the difference of expression of a nucleic acid, when compared to a normal standard sample is based on a statistical analysis of triplicate array data points using, for example, a student's t-test. “Differential expression” of a polynucleotide sequence, as used herein, is established if the expression of a sequence measured in several types of animal pain model, such as nerve injury models or an inflammation model, is increased or decreased by at least 1.2 fold in at least one of the pain models, and if the differential expression is found to be significant across three replicate analyses of differential expression in an animal pain model. Alternatively, a differentially expressed polynucleotide may be differentially expressed in several (e.g., two or more) animal pain models.

The “level of expression” is measured by hybridization analysis using labeled target nucleic acids according to methods well known in the art (see, for example, Ausubel et al., Short Protocols in Molecular Biology, 3^{rd} Ed. 1995, John Wiley and Sons, Inc.). The label on the target nucleic acid is a luminescent label, an enzymatic label, a radioactive label, a chemical label or a physical label. Preferably, the target nucleic acids are labeled with a fluorescent molecule. Preferred fluorescent labels include fluorescein, amino coumarin acetic acid, tetramethylrhodamine isothiocyanate (TRITC), Texas Red, Cy3 and Cy5.

As used herein, “differential expression” when measured using microarray hybridization as described herein, can be determined using one or more of three alternate measurements: (1) The hybridization intensity can be measured by comparing the level of hybridization of nucleic acid samples obtained from a naïve animal to the level of hybridization of nucleic acid samples from an animal subjected to any of the pain models described herein. This measurement is termed the “intensity ratio”. (2) Alternatively, a method of measuring “differential expression” is to utilize the “Affymetrix ratio” which is obtained by analyzing the hybridization levels obtained from nucleic acid samples obtained from a naïve animal and those obtained from nucleic acid samples obtained from an animal subjected to any of the pain models described herein, using the software provided with the Affymetrix Microarray software suite (Affymetrix, Santa Clara, Calif.). The Affymetrix ratio can be determined by following the protocols included with the Affymetrix brand software and microarray analysis equipment. Whether measured using the intensity ratio or the Affymetrix ratio, a nucleic acid molecule of the present invention is differentially expressed if it demonstrates at least a 1.4 fold change in expression levels in an animal subjected to the neuropathic or inflammation pain as described herein relative to an animal not subjected to the same pain. (3) Preferably, “differential expression” is measured in either a nerve injury model, or inflammation pain model, or both, at multiple time points after an animal has been subjected to pain. “Differential expression” is further measured in at least three replicate samples for each time point, and for multiple pain models (e.g. nerve injury models, an inflammation models), such that a statistical evaluation may be made of the significance of the differential expression. Accordingly, a polynucleotide sequence is “differentially expressed” if it is differentially expressed by at least 1.4 fold, and/or is differentially expressed by at least 1.2 fold, with a p-value of less than 0.05 across at least two sets of triplicate assays in any of the pain models versus the corresponding control. The fold differential expression, when paired with the statistical analysis of at least two replicate expression assays, can be measured using either of the “intensity ratio” or “affymetrix ratio” described above.

### DESCRIPTION OF THE DRAWINGS

FIG. 1 shows the data from a representative Northern analysis performed on target nucleic acid obtained from dorsal root ganglion neurons from a rat axotomy pain model.

FIG. 2 shows the in situ hybridization of dorsal root ganglion tissue sections with labeled oligonucleotide probes specific for SNAP, c-jun, or TrkA.

FIG. 3 shows the in situ hybridization of dorsal root ganglion tissue sections with labeled oligonucleotide probes specific for GTPcylco, IES-JE, CCHL2A, or VGF.

### DETAILED DESCRIPTION

The present invention is based, in part, on the discovery that the polynucleotides listed in Tables 1, 2, 3, 4, or 5 are differentially expressed by at least +/−1.4 fold in nerve injury and/or inflammation animal pain models. While the polynucleotides listed in Table 1 have been previously suggested to be regulated in pain models, the present invention is distinguished over the prior art in that only polynucleotides which demonstrate at least a +/−1.4 fold change in expression in a neuropathic and/or inflammation animal pain model are considered to be differentially expressed according to the invention. The invention further provides the polynucleotides listed in Tables 2, 3, 4, or 5 which are differentially expressed by at least +/−1.4 fold in a nerve injury or inflammation animal pain model, but which have not previously been suggested to be regulated in animal pain models (i.e., which are not indicate in Table 1). In addition, the invention provides the polynucleotides listed in Table 2 which have been identified herein as being differentially expressed by at least +/−1.2 fold at least two sets of triplicate assays in multiple nerve injury and inflammation pain models, with a p-value of less than 0.05. The invention further provides methods for identifying nucleic acid sequences which are differentially regulated in animals that have been subjected to pain, wherein differential expression is defined as an increase or decrease of the expression of the nucleic acid sequence by at least 1.2 fold compared to the same sequence in an animal which has not been subjected to pain, in triplicate assays with a statistical significance of p<0.05. The invention further provides methods for identifying nucleic acid sequences which are differentially regulated in animals that have been subjected to pain, wherein differential expression is defined as an increase or decrease of the expression of the nucleic acid sequence by at least 1.4 fold compared to the same sequence in an animal which has not been subjected to pain. The invention further provides methods of constructing arrays comprising isolated nucleic acid sequences which are differentially regulated in pain, and methods of screening for potential therapeutic compounds which may alter the expression of these sequences using the arrays. The invention also relates to methods for screening for candidate compounds which are capable of regulating the expression of one or more of the polynucleotide sequences of Tables 1, 2, 3, 4, or 5, or which are capable of regulating the activity of one or more of the polypeptides indicated in Table 1, 2, 3, 4, or 5, or a polypeptide encoded by one or more of the polynucleotides indicated in Table 1, 2, 3, 4, or 5, or which are capable of modulating pain in an animal.

In addition, the invention is based on the discovery that a subset of genes described in Tables 1-5 are differentially expressed in two or more neuropathic pain models. This subset of genes is shown in Table 10. The invention also relates to a yet smaller subset of genes than that of Table 10, which, based on their known or putative function in biological systems are likely to modulate or transduce pain by changing the properties, excitability, structure and/or connectivity of neurons in sensory pain circuits.

As described above, animals which have been subjected to pain include animal models of pain, in which the animal has been artificially manipulated to mimic one or more types of pain, including physiological, inflammatory, or neuropathic pain. Animals subjected to pain also include animals which have experienced pain as the result of a traumatic injury, or animals which have experienced physiological, inflammatory, or neuropathic pain not induced in the setting of an animal model.

Pain

The present invention relates to polynucleotides which are differentially expressed in (a) an animal that is subjected to pain relative to (b) an animal not subjected to pain. According to the invention, the pain to which the animals of (a) and (b) are subjected is the same pain, that is, if a polynucleotide is differentially expressed in an axotomy pain model then the differential expression is relative to the expression of the polynucleotide in an animal which is not an axotomy pain model.

As used herein, “pain” refers to a state-dependent sensory experience generated by the activation of peripheral sensory neurons, the nociceptors. As used herein, “pain” refers to several different types of pain, including physiological or protective pain, inflammatory pain that occurs after tissue damage, and neuropathic pain which occurs after damage to the nervous system. Physiological pain is initiated by sensory nociceptor fibers innervating the peripheral tissues and activated only by noxious stimuli, and is characterized by a high threshold to mechanical and thermal stimuli and rapid, transient responses to such stimuli. Inflammatory and neuropathic pain are characterized by displays of behavior indicating either spontaneous pain, measured by spontaneous flexion, vocalization, biting, or even self mutilation, or abnormal hypersensitivity to normally innocuous stimuli or to noxious stimuli, such as mechanical or thermal stimuli. Regardless of the type of pain, as used herein “pain” can be measured using behavioral criteria, such as thermal and mechanical sensitivity, weight bearing, visceral hypersensitivity, or spontaneous locomotor activity, electrophysiological criteria, such as in vivo or in vitro recordings from primary sensory neurons and central neurons to assess changes in receptive field properties, excitability or synaptic input, or neurochemical criteria, such as changes in the expression or distribution of neurotransmitters, neuropeptides and proteins in primary sensory and central neurons, activation of signal transduction cascades, expression of transcription factors, or phosphorylation of proteins.

Behavioral criteria used to measure “pain” include, but are not limited to mechanical allodynia and hyperalgesia, and temperature allodynia and hyperalgesia. Mechanical allodynia is generally measured using a series of ascending force von Frey monofilaments. The filaments are each assigned a force which must be applied longitudinally across the filament to produce a bend, or bow in the filament. Thus the applied force which causes an animal to withdraw a limb can be measured (Tal and Bennett, 1994 Pain 57: 375). An animal can be said to be experiencing “pain” if the animal demonstrates a withdrawal reflex in response to a force that is reduced by at least 30% compared to the force that elicits a withdrawal reflex in an animal which is not in “pain”. In one embodiment, an animal is said to be experiencing “pain” if the withdrawal reflex in response to a force that is reduced 40%, 50%, 60%, 70%, 80%, 90% and as much as 99% compared to the force required to elicit a similar reflex in a naïve animal.

Mechanical hypersensitivity can be measured by applying a sharp object, such as a pin, to the skin of an animal with a force sufficient to indent, but not penetrate the skin. The duration of withdrawal from the sharp stimulus may then be measured, wherein an increase in the duration of withdrawal is indicative of “pain” (Decostard et al., 1998 Pain 76: 159). For example, an animal can be said to be experiencing “pain” if the withdrawal duration following a sharp stimulus is increased by at least 2 fold compared with an animal that is not experiencing “pain”. In one embodiment, an animal is said to be experiencing “pain” if the withdrawal duration is increased by 3, 4, 5, 6, 7, 8, 9, and up to 10 fold compared to an animal not experiencing “pain”.

Temperature allodynia can be measured by placing a drop of acetone onto the skin surface of an animal using an instrument such as a blunt needle attached to a syringe without touching the skin with the needle. The rapid evaporation of the acetone cools the skin to which it is applied. The duration of the withdrawal response to the cold sensation can then be measured (Choi et al., 1994 Pain 59: 369). An animal can be said to be in “pain” if the withdrawal duration following acetone application is increased by at least 2 fold as compared to an animal that is not experiencing “pain”. According to the invention an animal can be said to be in “pain” if the withdrawal duration following thermal stimulation is increased by 4, 6, 8, 10, 12, 14, 16, 18, and up to 20 fold compared to an animal not experiencing “pain”.

Temperature hyperalgesia can be measured by exposing a portion of the skin surface of an animal, such as the plantar surface of the foot, to a beam of radiant heat through a transparent perspex surface (Hargreaves et al., 1988 Pain 32:77). The duration of withdrawal from the heat stimulus may be measured, wherein an increase in the duration of withdrawal is indicative of “pain”. An animal can be said to be experiencing “pain” if the duration of the withdrawal from the heat stimulus increases by at least 2 fold compared with an animal that is not experiencing “pain”. In addition, an animal can be said to be experiencing “pain” if the duration of the withdrawal from heat stimulus is increased by 3, 4, 5, 6, 7, 8, 9, and up to 10 fold compared with an animal that is not experiencing “pain”.

In addition to the behavioral criteria described above, an animal can be deemed to be experiencing “pain” by measuring electrophysiological changes, in vitro or in vivo, in primary sensory, or central sensory neurons. Electrophysiological changes can include increased neuronal excitability, changes in receptive field input, or increased synaptic input. The technique of measuring cellular physiology is well known to those of skill in the art (see, for example, Hille, 1992 Ion channels of excitable membranes. Sinauer Associates, Inc., Sunderland, Mass.). An increase in neuronal excitability may be identified, for example, by measuring an increase in the number of action potentials per unit time in a given neuron. An animal is said to be experiencing “pain” if there is at least a 2 fold increase in the action potential firing rate compared with an animal that is not experiencing “pain.” In addition, and animal can be said to be experiencing “pain” if the action potential firing rate is increased by, 3, 4, 5, 6, 7, 8, and up to 10 fold compared to an animal that is not experiencing “pain”. An increase in synaptic input to a sensory neuron, either peripheral or central, may be identified, for example, by measuring the rate of end-plate excitatory potentials (EPSPS) recorded in from the neuron. An animal is said to be experiencing “pain” if there is at least a 2 fold, 3, 4, 5, 6, 7, 8, and up to 10 fold increase in the rate of EPSPs recorded from a given neuron compared to an animal that is not experiencing pain.

Alternatively, neurochemical criteria may be used to determine whether or not an animal is experiencing “pain”. For example, an animal which has experienced “pain” will display changes in the expression or distribution of neurotransmitters, neuropeptides and protein in primary sensory and central neurons, activation of signal transduction cascades, expression of transcription factors, or phosphorylation of proteins. Gene and protein expression, and phosphorylation of proteins such as transcription factors may be measured using a number of techniques known to those of skill in the art including but not limited to PCR, Southern analysis, Northern analysis, Western analysis, immunohistochemistry, and the like. Examples of signal transduction pathway constituents which may be activated in an animal which is experiencing pain include, but are not limited to ERK, p38, and CREB. Examples of genes which may exhibit enhanced expression include immediate early genes such as c-fos, protein kinases such as PKC and PKA. Examples of other proteins which may be phosphorylated in an animal experiencing pain include receptors and ion channels such as the NMDA or AMPA receptors. Regardless of whether the measure is of transcription, translation or phosphorylation an animal can be said to be experiencing “pain” if one measures at least a 2 fold increase or decrease in any of these parameters compared to an animal not experiencing pain. An animal can be further said to be experiencing “pain” if there is a 3, 4, 5, 6, 7, 8, and up to 10 fold increase in the measurement of any of the above parameters compared to an animal not experiencing “pain”.

As used herein, “pain” refers to any of the behavioral, electrophysiological, or neurochemical criteria described above. In addition, “pain” can be assessed using combinations of these criteria.

As used herein, “pain” can refer to “pain” experienced by an animal as a result of accidental trauma (e.g., falling trauma, burn trauma, toxic trauma, etc.), congenital deformity or malformation, infection (e.g., inflammatory pain), or other conditions which are not within the control of the animal experiencing the “pain”. Alternatively, “pain” may be inflicted onto an animal by subjecting the animal to one or more “pain models”.

The present invention comprises polynucleotide sequences that are differentially expressed in nerve injury pain models, including axotomy, SNI, chronic constriction, and segmental nerve lesion, as well as inflammation pain models. It is also within the scope of the present invention that the polynucleotides described herein as being differentially expressed in nerve injury, or neuropathic pain models may be also differentially expressed in other pain models known to those of skill in the art.

As used herein, a “pain model” refers to any manipulation of an animal during which the animal experiences “pain”, as defined above. “Pain models” can be classified as those that test the sensitivity of normal animals to intense or noxious stimuli. These tests include responses to thermal, mechanical, or chemical stimuli. Thermal stimuli is usually hot (42 to 55° C.) and includes radiant heat to the tail (the tail flick test) radiant heat to the plantar surface of the hindpaw (the Hargreaves test, supra), the hotplate test, and immersion of the hindpaw or tail in hot water. Alternatively, thermal stimuli can be cold stimulus (30° to −10° C.), such as immersion in cold water, acetone evaporation or cold plate tests which may be used to test cold pain responsiveness using the thresholds discussed above. The end points are latency to response and the duration of the response as well as vocalization and licking the paw, as described above. Mechanical Stimuli typically involves measurements of the threshold for eliciting a withdrawal reflex of the hindpaw to graded strength monofilament von Frey hairs wherein one can measure the force of the filament required to elicit a reflex. Alternatively, mechanical stimuli can be a sustained pressure stimulus to a paw (e.g., the Ugo Basila analgesiometer). The duration of response to a standard pin prick can also be measured. Threshold values for identifying a stimulus that causes “pain” to the animal are described above. Chemical Stimuli typically involves the application or injection of a chemical irritant to the skin, muscle joints or internal organs like the bladder or peritoneum. Irritants can include capsaicin, mustard oil, bradykinin, ATP, formalin, or acetic acid. The outcome measures include vocalization, licking the paw, writhing or spontaneous flexion.

Alternatively, a “pain model” can be a test that measures changes in the excitability of the peripheral or central components of the pain neural pathway pain sensitization, termed “peripheral sensitization” and “central sensitization”. “Peripheral Sensitization” involves changes in the threshold and responsiveness of high threshold nociceptors which can be induced by: repeated heat stimuli, or application or injection of sensitizing chemicals (e.g. prostaglandins, bradykinin, histamine, serotonin, capsaicin, mustard oil). The outcome measures are thermal and mechanical sensitivity in the area of application/stimulation using the techniques described above in behaving animals or electrophysiological measurements of single sensory fiber receptive field properties either in vivo or using isolated skin nerve preparations. “Central sensitization” involves changes in the excitability of neurons in the central nervous system induced by activity in peripheral pain fibers. “Central sensitization” can be induced by noxious stimuli (e.g., heat) chemical irritants (e.g., injection/application of capsaicin/mustard oil or formalin or electrical activation of sensory fibers). The outcome measures are: behavioral, electrophysiological, and neurochemical.

Alternatively, a “pain model” can refer to those tests that measure the effect of peripheral inflammation on pain sensitivity. The inflammation can be produced by injection of an irritant such as complete Freunds adjuvant, carrageenan, turpentine, croton oil etc into the skin, subcutaneously, into a muscle into a joint or into a visceral organ. Production of a controlled UV light burn and ischaemia can also be used. Administration of cytokines or inflammatory mediators such as lipopolysaccharide (LPS), or nerve growth factor (NGF) can mimic the effects of inflammation. The outcome of these models may also be measured as behavioral, electrophysiological, and/or neurochemical changes.

Further, a “pain model” includes those tests that mimic peripheral neuropathic pain using lesions of the peripheral nervous system. Examples of such lesions include, but are not limited to complete transection of a peripheral nerve (axotomy; Watson, 1973, J. Physiol. 231:41), liagation of a spinal segmental nerve (CHUNG model; Kim and Chung, 1992, Pain, 50:355-63), partial nerve injury (Seltzer, 1979, Pain, 29: 1061), Spared Nerve Injury model (Decosterd and Woolf, 2000, Pain 87:149), chronic constriction injury (Bennett, 1993 Muscle Nerve 16: 1040), toxic neuropathies, such as diabetes (streptozocin model), pyridoxine neuropathy, taxol, vincristine and other antineoplastic agent-induced neuropathies, ischaemia to a nerve, peripheral neuritis models (e.g., CFA applied perineurally), models of postherpetic neuralgia using HSV infection. Such neuropathic pain models are also referred to herein as a “nerve injury pain model”. The outcome of these neuropathic or nerve injury “pain models” can be measured using behavioral, electrophysiological, and/or neurochemical criteria as described above.

In addition, a “pain model” refers to those tests that mimic central neuropathic pain using lesions of the central nervous system. For example, central neuropathic pain may be modeled by mechanical compressive, ischemic, infective, or chemical injury to the spinal cord of an animal. The outcome of such a model is measured using the behavioral, electrophysiological, and/or neurochemical criteria described above.

Identification of Nucleic Acid Sequences Differentially Expressed in Pain

In one embodiment, the present invention provides isolated nucleic acid sequences which are differentially regulated in an animal which has been subjected to neuropathic pain relative to an animal not subjected to neuropathic pain, and a method for identifying such sequences. The present invention provides a method for identifying a nucleotide sequence which is differentially regulated in an animal subjected to pain, comprising: hybridizing a nucleic acid sample corresponding to RNA obtained from the animal to a nucleic acid sample comprising one or more nucleic acid molecules of known identity; and measuring the hybridization of the nucleic acid sample to the one or more nucleic acid molecules of known identity, wherein a 1.4 fold difference in the hybridization of the nucleic acid sample to the one or more nucleic acid molecules of known identity relative to a nucleic acid sample obtained from an animal which has not been subjected to the same pain is indicative of the differential expression of the nucleotide sequence in an animal subjected to pain. Alternatively, the invention provides a method for identifying a nucleotide sequence which is differentially regulated in an animal subjected to pain, comprising: hybridizing at least three replicates of a nucleic acid sample corresponding to RNA obtained from the animal to at least three replicates of a nucleic acid sample comprising one or more nucleic acid molecules of known identity and measuring the hybridization of the nucleic acid sample to the one or more nucleic acid molecules of known identity for each of said replicates. A 1.2 fold difference in the hybridization, and a p-value of less than 0.05 across the replicates, of the nucleic acid sample to the one or more nucleic acid molecules of known identity relative to a nucleic acid sample obtained from an animal which has not been subjected to pain is indicative of the differential expression of the nucleotide sequence in the animal subjected to pain

Generally, the present invention provides a method for identifying nucleic acid sequences which are differentially regulated in an animal which has been subjected to pain comprising isolating messenger RNA from an animal, generating cRNA from the mRNA sample, hybridizing the cRNA to a microarray comprising a plurality of nucleic acid molecules stably associated with discrete locations on the array, and identifying patterns of hybridization of the cRNA to the array. According to the present invention, a nucleic acid molecule which hybridizes to a given location on the array is said to be differentially regulated if the hybridization signal is at least 1.4 fold higher or lower than the hybridization signal at the same location on an identical array hybridized with a nucleic acid sample obtained from an animal that has not been subjected to pain. Alternatively, at least three independent replicate RNA samples are generated and hybridized to at least three replicate arrays, such that statistical significance may be conferred to the fold change in expression of a sequence in an animal subjected to pain relative to an animal not subjected to pain, wherein a 1.2 fold change in expression and a p-value of less than 0.05 is indicative of differential expression.

Nucleic Acid Samples

Nucleic acid samples to be examined for differentially regulated sequences may be obtained from animals using techniques that are well described in the art. In a preferred embodiment of the invention, the animal from which the nucleic acid is obtained is a pain model. In one embodiment, an animal pain model is an experimental model which tests the sensitivity of normal animals to intense or noxious stimuli. These tests include responses to thermal, mechanical, or chemical stimuli. Thermal stimuli is usually hot (42 to 55° C.) and includes radiant heat to the tail (the tail flick test) radiant heat to the plantar surface of the hindpaw (the Hargreaves test, supra), the hotplate test, and immersion of the hindpaw or tail in hot water. Alternatively, thermal stimuli can be cold stimulus (30° to −10° C.), such as immersion in cold water, acetone evaporation or cold plate tests which may be used to test cold pain responsiveness using the thresholds discussed above. The end points are latency to response and the duration of the response as well as vocalization and licking the paw, as described above. Mechanical stimuli typically involves measurements of the threshold for eliciting a withdrawal reflex of the hindpaw to graded strength monofilament von Frey hairs wherein one can measure the force of the filament required to elicit a reflex. Alternatively, mechanical stimuli can be a sustained pressure stimulus to a paw (e.g., the Ugo Basila analgesiometer). The duration of response to a standard pin prick can also be measured. Threshold values for identifying a stimulus that causes “pain” to the animal are described above. Chemical Stimuli typically involves the application or injection of a chemical irritant to the skin, muscle joints or internal organs like the bladder or peritoneum. Irritants can include capsaicin, mustard oil, bradykinin, ATP, formalin, or acetic acid. The outcome measures include vocalization, licking the paw, writhing or spontaneous flexion. In an alternate embodiment, the animal pain model is designed to measure changes in the excitability of the peripheral or central components of the pain neural pathway pain sensitization, termed peripheral sensitization and central sensitization. Peripheral Sensitization involves changes in the threshold and responsiveness of high threshold nociceptors which can be induced by: repeated heat stimuli, or application or injection of sensitizing chemicals (e.g. prostaglandins, bradykinin, histamine, serotonin, capsaicin, mustard oil). The outcome measures are thermal and mechanical sensitivity in the area of application/stimulation using the techniques described above in behaving animals or electrophysiological measurements of single sensory fiber receptive field properties either in vivo or using isolated skin nerve preparations. Central sensitization involves changes in the excitability of neurons in the central nervous system induced by activity in peripheral pain fibers. Central sensitization can be induced by noxious stimuli (e.g., heat) chemical irritants (e.g., injection/application of capsaicin/mustard oil or formalin or electrical activation of sensory fibers). The outcome measures are: behavioral, electrophysiological, and neurochemical. In a further embodiment, the animal pain model is an experimental model that measures the effect of peripheral inflammation on pain sensitivity. The inflammation can be produced by injection of an irritant such as complete Freunds adjuvant, carrageenan, turpentine, croton oil etc into the skin, subcutaneously, into a muscle into a joint or into a visceral organ using doses and administration techniques that are well known in the art. Production of a controlled UV light burn and ischaemia can also be used. Administration of cytokines or inflammatory mediators such as lipopolysaccharide (LPS), or nerve growth factor (NGF) can mimic the effects of inflammation. The outcome of these models may also be measured as behavioral, electrophysiological, and/or neurochemical changes.

In a preferred embodiment, the animal pain model is a model that mimic peripheral neuropathic pain using lesions of the peripheral nervous system (i.e., a nerve injury model). Examples of such lesions include, but are not limited to complete transection of a peripheral nerve (axotomy; Watson, 1973, J. Physiol. 231:41), liagation of a spinal segmental nerve (Kim and Chung, 1992, Pain, 50:355-63), partial nerve injury (Seltzer, 1979, Pain, 29: 1061), Spared Nerve Injury model (Decosterd and Woolf, 2000, Pain 87:149), chronic constriction injury (Bennett, 1993 Muscle Nerve 16: 1040), toxic neuropathies, such as diabetes (streptozocin model), pyridoxine neuropathy, taxol, vincristine and other antineoplastic agent-induced neuropathies, ischaemia to a nerve, peripheral neuritis models (e.g., CFA applied perineurally), models of postherpetic neuralgia using HSV infection. The outcome of these neuropathic pain models can be measured using behavioral, electrophysiological, and/or neurochemical criteria as described above. Alternatively, the neuropathic animal pain model may be one which mimics central neuropathic pain using lesions of the central nervous system. For example, central neuropathic pain may be modeled by mechanical compressive, ischemic, infective, or chemical injury to the spinal cord of an animal. The outcome of such a model is measured using the behavioral, electrophysiological, and/or neurochemical criteria described above.

In a further preferred embodiment, the animal pain model is a model which mimics inflammation using injectable irritants and/or inflammatory mediators. Examples of such models include animals which are injected with, for example complete Freunds adjuvant (CFA), carrageenan, turpentine, croton oil, cytokines, lippopolysoccharide (LPS), or nerve growth factor (NGF) (Stein et al., 1988 Pharmacol Biochem Behav 31:445; Woolf et al., 1994, Neuroscience, 62: 327). The outcome of inflammation pain model can be measured using behavioral, electrophysiological, and/or neurochemical criteria as described above.

Alternatively, nucleic acid samples may be obtained from animals which are not pain models, but which have been subjected to pain as a result of traumatic injury, infection, genetic, or congenital birth defects, and the like. In addition, nucleic acid samples may be obtained from an animal which is not a pain model, and which has not been subjected to pain as a result of a traumatic injury, or infection. Such an animal is termed a “naïve” animal, and the expression of nucleic acid sequences in the naïve animal can be compared to the expression of the same nucleic acid molecules in animals subjected to pain to determine differential expression.

Nucleic acid samples, useful in the present invention for determining differential expression of nucleic acid sequences in an animal subjected to pain may be obtained from any cell of the animal. In a preferred embodiment, the nucleic acid is obtained from one or more sensory neurons of the animal. In a further preferred embodiment the nucleic acid is obtained from the primary sensory neurons of the dorsal root ganglion or dorsal horn of the spinal cord. However, nucleic acid may be obtained from other neurons including, but not limited to cranial nerve nuclei, peripheral and/or central autonomic neurons, enteric neurons, thalamic neurons, and neurons of sensory regions of the cortex such as primary sensory cortex.

Sensory neurons may be obtained from an animal using techniques that are well established in the art. For example, in embodiments where nucleic acid samples are to be obtained from rat dorsal root ganglion (DRG) neurons, rats (whether naïve or pain models) are rapidly killed by decapitation and the DRG is dissected, removed and quickly snap-frozen on a bed of crushed dry ice, or in liquid nitrogen. RNA is then extracted from the tissues, also using techniques that are well known in the art (see, for example, Ausubel supra). For example, the tissue is prepared by homogenization in a glass teflon homogenizer in 1 ml denaturing solution (4M guanidinium thiosulfate, 25 mM sodium citrate, pH 7.0, 0.1M 2-ME, 0.5% (w/v) N-laurylsarkosine) per 100 mg tissue. Following transfer of the homogenate to a 5-ml polypropylene tube, 0.1 ml of 2 M sodium acetate, pH 4, 1 ml water-saturated phenol, and 0.2 ml of 49:1 chloroform/isoamyl alcohol are added sequentially. The sample is mixed after the addition of each component, and incubated for 15 min at 0-4° C. after all components have been added. The sample is separated by centrifugation for 20 min at 10,000×g, 4° C., precipitated by the addition of 1 ml of 100% isopropanol, incubated for 30 minutes at −20° C. and pelleted by centrifugation for 10 minutes at 10,000×g, 4° C. The resulting RNA pellet is dissolved in 0.3 ml denaturing solution, transferred to a microfuge tube, precipitated by the addition of 0.3 ml of 100% isopropanol for 30 minutes at −20° C., and centrifuged for 10 minutes at 10,000×g at 4° C. The RNA pellet is washed in 70% ethanol, dried, and resuspended in 100-200 μl DEPC-treated water or DEPC-treated 0.5% SDS (Chomczynski and Sacchi, 1987, Anal. Biochem., 162: 156).

Alternatively, total RNA may be extracted from tissues useful in the present invention using Trizol reagent (Invitrogen, Carlsbad, Calif.), following the manufacturers instructions. Purity and integrity of RNA is assessed by absorbance at 260/280 nm and separation of RNA samples on a 1% agarose gel followed by inspection under ultraviolet light.

Following total RNA isolation from tissues or cell of an animal useful in the present invention, the RNA is converted to cRNA for use in array hybridization. The preparation of cRNA is well-known and well-documented in the prior art.

In an alternate embodiment, the total RNA is converted to cDNA for use in array hybridization. cDNA may be prepared according to the following method. Total cellular RNA is isolated (as described) and passed through a column of oligo(dT)-cellulose to isolate polyA RNA. The bound polyA mRNAs are eluted from the column with a low ionic strength buffer. To produce cDNA molecules, short deoxythymidine oligonucleotides (12-20 nucleotides) are hybridized to the polyA tails to be used as primers for reverse transcriptase, an enzyme that uses RNA as a template for DNA synthesis. Alternatively, mRNA species are primed from many positions by using short oligonucleotide fragments comprising numerous sequences complementary to the mRNA of interest as primers for cDNA synthesis. The resultant RNA-DNA hybrid is converted to a double stranded DNA molecule by a variety of enzymatic steps well-known in the art (Watson et al., 1992, Recombinant DNA, 2nd edition, Scientific American Books, New York).

Microarray Analysis

In one embodiment, the present invention provides a method for the identification of differentially expresses nucleic acid sequences in pain in which cDNA obtained from sensory neurons of animals subjected to pain is hybridized to a polynucleotide microarray of known genes or ESTs and the hybridization levels of the cDNA to the polynucleotide microarray are measured.

Microarrays, useful in the identification of differentially expressed nucleic acid sequences, may be any microarray known in the art which comprises known sequences. A polynucleotide microarray refers to a plurality of unique nucleic acids attached to one surface of a solid support at a density exceeding 20 different nucleic acids/cm² wherein each of the nucleic acids is attached to the surface of the solid support in a non-identical preselected region. In one embodiment, the nucleic acid attached to the surface of the solid support is DNA. In a preferred embodiment, the nucleic acid attached to the surface of the solid support is cDNA. In another preferred embodiment, the nucleic acid attached to the surface of the solid support is cDNA synthesized by polymerase chain reaction (PCR). Preferably, a nucleic acid comprising an array, according to the invention, is at least 20 nucleotides in length. Preferably, a nucleic acid comprising an array is less than 6,000 nucleotides in length. More preferably, a nucleic acid comprising an array is less than 500 nucleotides in length. In one embodiment, the array comprises at least 500 different nucleic acids attached to one surface of the solid support. In another embodiment, the array comprises at least 10 different nucleic acids attached to one surface of the solid support. In yet another embodiment, the array comprises at least 10,000 different nucleic acids attached to one surface of the solid support.

In a preferred embodiment, the microarray comprises known nucleic acid molecules stably associated with discrete predefined regions, and which are obtained from an animal of the same species as the animal which had been subjected to pain and from which the nucleic acid sample to be tested is obtained. In a preferred embodiment, the microarray is a commercially available microarray which may be obtained from a commercial source such as Affymetrix (Santa Clara, Calif.). For example, in one embodiment nucleic acid samples are obtained from a rat pain model and are hybridized to a polynucleotide microarray comprising known rat gene sequences and ESTs. In a further preferred embodiment, the microarray is an Affymetrix Gene Chip® array including, but not limited to the human U95 array, the murine U74 array, and the rat U34 array.

In one embodiment three independent replicate nucleic acid samples are prepared from three separate pain model animals (for tissues with a low abundance of nerve cells, such as the DRG, samples from several animals may be pooled to generate a single replicate) are hybridized to at least three replicate polynucleotide arrays, such that a statistical analysis may be performed on the resulting hybridization levels.

Sample Preparation

Prior to hybridization of nucleic acid to the polynucleotide microarray, the nucleic acid samples must be prepared to facilitate subsequent detection of hybridization. The nucleic acid samples obtained from animals that have been subjected to pain (and from naïve animals for the determination of differential expression) are referred to as “probes” for the microarray and are capable of binding to a polynucleotide or nucleic acid member of complementary sequence through one or more types of chemical bonds, usually through complementary base pairing, usually through hydrogen bond formation.

As used herein, a polynucleotide derived from an mRNA transcript refers to a polynucleotide for which synthesis of the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable target nucleic acid samples include, but are not limited to, mRNA transcripts of a gene or genes, cDNA reverse transcribed from the mRNA, cRNA transcribed from the cDNA, DNA amplified from a gene or genes, RNA transcribed from amplified DNA, and the like. The polynucleotide probes used herein are preferably derived from sensory neurons of an animal that has been subjected to pain.

In the simplest embodiment, such a polynucleotide probe comprises total mRNA or a nucleic acid sample corresponding to mRNA (e.g., cDNA) isolated from sensory neurons, ganglia, nuclei, or brain tissue. In another embodiment, the total mRNA is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987). In a preferred embodiment, total RNA is extracted using TRIzol reagent (GIBCO/BRL). Purity and integrity of RNA is assessed by absorbance at 260/280 nm and agarose gel electrophoresis followed by inspection under ultraviolet light.

In some embodiments, it is desirable to amplify the probe nucleic acid sample prior to hybridization, for example, when total RNA is obtained from a small population of neurons. One of skill in the art will appreciate that whatever amplification method is used, if a quantitative result is desired, care must be taken to use a method that maintains or controls for the relative frequencies of the amplified polynucleotides. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified polynucleotide. Detailed protocols for quantitative PCR are provided in PCR Protocols, A Guide to Methods and Applications, innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the probe nucleic acid sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material thereby permitting expression monitoring even where biological samples are limited.

In order to measure the hybridization of a probe nucleic acid to a polynucleotide array to determine differential expression, the probe nucleic acid is preferable labeled with a detectable label. Any analytically detectable marker that is attached to or incorporated into a molecule may be used in the invention. An analytically detectable marker refers to any molecule, moiety or atom which is analytically detected and quantified.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, 35S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The labels may be incorporated by any of a number of means well known to those of skill in the art. However, in a preferred embodiment, the label is simultaneously incorporated into the probe during the amplification step in the preparation of the probe polynucleotides. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed polynucleotides.

Alternatively, a label may be added directly to the original polynucleotide sample (e.g., mRNA, polyA mRNA, cDNA, etc.) or to the amplification product after the amplification is completed. Means of attaching labels to polynucleotides are well known to those of skill in the art and include, for example nick translation or end-labeling (e.g. with a labeled RNA) and subsequent attachment (ligation) of a polynucleotide linker joining the sample polynucleotide to a label (e.g., a fluorophore).

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013.).

In a preferred embodiment, a probe nucleic acid obtained from an animal that has been subjected to pain and a nucleic acid sample obtained from an animal not subjected to pain are co-hybridized to the polynucleotide array. In this embodiment, the two probe samples used for comparison are labeled with different fluorescent dyes which produce distinguishable detection signals, for example, probes made from an animal pain model are labeled with Cy5 and probes made from a naïve animal are labeled with Cy3. The differently labeled target samples are hybridized to the same microarray simultaneously. In a preferred embodiment, the labeled targets are purified using methods known in the art, e.g., ethanol purification or column purification.

In a preferred embodiment, the probes will include one or more control molecules which hybridize to control sequences on the microarray to normalize signals generated from the microarray. Labeled normalization targets are polynucleotide sequences that are perfectly complementary to control oligonucleotides that are spotted onto the microarray. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Preferred normalization probes are selected to reflect the average length of the other probes present in the sample, however, they are selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any other probe molecules.

Hybridization to Polynucleotide Arrays

To determine the differential expression of a nucleic acid sequence in an animal subjected to pain, labeled probe nucleic acids are hybridized to a polynucleotide array comprising polynucleotides of known sequence or identity. Polynucleotide hybridization involves providing a denatured probe and target polynucleotide under conditions where the probe nucleic acid member and its complementary target can form stable hybrid duplexes through complementary base pairing. The polynucleotides that do not form hybrid duplexes are then washed away leaving the hybridized polynucleotides to be detected, typically through detection of an attached detectable label. It is generally recognized that polynucleotides are denatured by increasing the temperature or decreasing the salt concentration of the buffer containing the polynucleotides. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

The invention provides for hybridization conditions comprising the Dig (digoxygenin) hybridization mix (Boehringer); or formamide-based hybridization solutions, for example as described in Ausubel et al., supra and Sambrook et al. supra.

Alternatively, as described above, a preferred embodiment of the present invention comprises hybridizing probe nucleic acid molecules to an Affymetrix Gene Chip®. In this embodiment, hybridization of the probe nucleic acid molecules to the polynucleotide array is carried out according to the manufacturers instructions.

Methods of optimizing hybridization conditions are well known to those of skill in the art (see, e.g., Laboratory Techniques in Biochemistry and Molecular Biology, Vol. 24: Hybridization With Polynucleotide Probes, P. Tijssen, ed. Elsevier, N.Y., (1993)).

Following hybridization, non-hybridized labeled or unlabeled polynucleotide is removed from the support surface, conveniently by washing, thereby generating a pattern of hybridized probe polynucleotide on the substrate surface. A variety of wash solutions are known to those of skill in the art and may be used. The resultant hybridization patterns of labeled, hybridized oligonucleotides and/or polynucleotides may be visualized or detected in a variety of ways, with the particular manner of detection being chosen based on the particular label of the test polynucleotide, where representative detection means include scintillation counting, autoradiography, fluorescence measurement, calorimetric measurement, light emission measurement and the like. In the preferred embodiment, in which the probe nucleic acid is hybridized to an Affymetrix Gene Chip®, the hybridization pattern of the probe nucleic acid molecules is detected and measured according to the Affymetrix protocol, and using Affymetrix instrumentation.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding to the signal emitted by a known number of end labeled target polynucleotides to obtain a count or absolute value of the copy number of each end-labeled target that is hybridized to a particular spot on the array in the hybridization pattern.

Expression Analysis

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the test polynucleotides from the remaining data. The resulting data is displayed as an image with the intensity in each region varying according to the binding affinity between associated oligonucleotides and/or polynucleotides and the test polynucleotides.

According to the present invention, there are three sets of measurements which may be used to determine differential expression of a polynucleotide obtained from an animal subjected to pain relative to an animal not subjected to pain. In one embodiment, differential expression may be determined by measuring the intensity ratio, as defined above, wherein a +/−1.4 fold change or greater in the intensity ratio is indicative of differential expression. In a preferred embodiment, differential expression may be determined by measuring the Affymetrix ratio using the software suite and manufacturers protocols, available from Affymetrix (Santa Clara, Calif.), wherein a change in expression of +/−1.4 fold or greater is indicative of differential expression.

In another preferred embodiment, differential expression of sequences can be established if they are differentially expressed by at least 1.2 fold, with a p-value of less than 0.05, in a statistical analysis of triplicate array data points using an appropriate statistical analysis, such as the student's t-test.

For example, Table 2 represents a composite of all those genes which were originally identified as differentially regulated by at least 1.4 fold in either SNI or axotomy pain models. Differential expression was subsequently evaluated in at least three replicate arrays using at least three replicate nucleic acid samples obtained from the animal nerve injury and inflammation pain models. From the replicate screening method, polynucletoide sequences can be identified as differentially expressed which have a lower fold change (i.e., lower than 1.4 fold) in expression in an animal subjected to pain, provided that a statistical analysis of the replicate data yields a p-value of less than 0.05. Tables 6 and 7 below show an example of an experimental replicate scheme which may be used to obtain the data shown in Table 2. The animal pain model is indicated in the column labeled “animal model”, and the elapsed time following the generation of the pain model (i.e., time post surgery) is indicated. Experiments can be performed on samples obtained from both dorsal horn (Table 6) and DRG (Table 7) tissues.

TABLE 6
Affimetrix microarray experiments

| Animal Model | Time Points | | | | # hybridization exp | Total # r. | |
|---|---|---|---|---|---|---|---|
| CCI DH | 3 d | 7 d | 21 d | 40 d | 4 × 3 | | 12 |
| Chung DH | 3 d | 7 d | 21 d | 40 d | 4 × 3 | | 12 |
| SNI DH | 3 d | 7 d | 21 d | 40 d | 4 × 3 | | 12 |
| Sham CCI = SNI DH | 3 d | 7 d | 21 d | none | 3 × 3 | | 9 |
| Sham Chung DH | 3 d | 7 d | 21 d | none | 3 × 3 | | 9 |
| Naïve DH | | | | | 1 × 3 | | 3 |
| | | | | | | Total | 57 |
| CFA injec. DH | 12 h | 24 h | 5 d | | 3 × 3 | | 9 |
| | | | | | | Total | 67 |

TABLE 7
Affimetrix microarray experiments

| | | | | | # hybridization |
|---|---|---|---|---|---|
| Animal Model | Time Points | | | | exp |
| CCI DRG L4 | 3 d | 7 d | 21 d | 40 d | 4 × 3 |
| Chung DRG L4 | 3 d | 7 d | 21 d | 40 d | 4 × 3 |
| SNI DRG L4 | 3 d | 7 d | 21 d | 40 d | 4 × 3 |
| CCI DRG L5 | 3 d | 7 d | 21 d | 40 d | 4 × 3 |
| Chung DRG L5 | 3 d | 7 d | 21 d | 40 d | 4 × 3 |
| SNI DRG L5 | 3 d | 7 d | 21 d | 40 d | 4 × 3 |
| Sham CCI = SNI L4 + L5 | 3 d | 7 d | 21 d | none | 3 × 3 |
| Sham Chung L4 + L5 | 3 d | 7 d | 21 d | none | 3 × 3 |
| Naïve L4 | | | | | 1 × 3 |
| Naïve L5 | | | | | 1 × 3 |
| CFA injec. DRG (L4 + L5 | 12 h | 24 h | 5 d | | 3 × 3 |
| pool) | | | | | |
| | | | | | Total 105 |

DH = dorsal horn of the spinal cord
DRG = dorsal root ganglion
CCI = chronic constriction of the sciatic nerve
Chung = ligation of the spinal nerves L5 and L6 (lombar region) distal to the correspondent dorsal root ganglions
SNI = spare nerve injury model (ligation and axotomy of the tibial and pereonal nerves)
CFA = injection in the paw of complete Freund's adijuvant (inflammatory pain model)

The nerve injury pain models represented are the Spinal segmental nerve injury (Chung), Chronic Constriction Injury (CCI) and Spared Nerve Injury (SNI) models at time points 3, 7, 21 and 40 days. The inflammatory model represented is intraplantar Complete Freund's Adjuvant (CFA) injection into the hind paw at 0.5, 1 and 5 days post injection. The tissue are lumbar DRGs and dorsal horn (i.e two tissues four models, 4 time points (3 for CFA)=30 different pain comparisons each in triplicate each compared against the appropriate control.

The following is an example of a detection protocol that may be used for the simultaneous analysis of two nucleic acid samples to be compared, wherein one sample is obtained from primary sensory neurons of an animal pain model and the other is obtained from primary sensory neurons of a naïve animal, and wherein each sample is labeled with a different fluorescent dye, such as Cy3 and Cy5. This type of protocol would produce an intensity ratio.

Each element of the microarray is scanned for the first fluorescent color. The intensity of the fluorescence at each array element is proportional to the expression level of that nucleic acid sequence in the sample.

The scanning operation is repeated for the second fluorescent label. The ratio of the two fluorescent intensities provides a highly accurate and quantitative measurement of the relative gene expression level in the two primary sensory neuron samples.

In a preferred embodiment, fluorescence intensities of the immobilized target nucleic acid sequences can be determined from images taken with a custom confocal microscope equipped with laser excitation sources and interference filters appropriate for the Cy3 and Cy5 fluorophores. Separate scans were taken for each fluorophore at a resolution of 225 μm² per pixel and 65,536 gray levels. Image segmentation to identify areas of hybridization, normalization of the intensities between the two fluorophore images, and calculation of the normalized mean fluorescent values at each target are as described (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013. Chen, Y., Dougherty, E. R. & Bittner, M. L. (1997) Biomed. Optics 2, 364374). Normalization between the images is used to adjust for the different efficiencies in labeling and detection with the two different fluorophores. This is achieved by equilibrating to a value of (1) the signal intensity ratio of a set of internal control genes spotted on the array.

Following detection or visualization, the hybridization pattern is used to determine quantitative information about the genetic profile of the labeled probe polynucleotide sample that was contacted with the array to generate the hybridization pattern, as well as the physiological source from which the labeled probe polynucleotide sample was derived. By genetic profile is meant information regarding the types of polynucleotides present in the sample, e.g. in terms of the types of genes to which they are complementary, as well as the copy number of each particular polynucleotide in the sample. From this data, one can also derive information about the physiological source from which the target polynucleotide sample was derived, such as the types of genes expressed in the tissue or cell which is the physiological source, as well as the levels of expression of each gene, particularly in quantitative terms.

In a particularly preferred embodiment, where it is desired to quantify the transcription level (and thereby expression) of one or more polynucleotide sequences in a sample, the probe nucleic acid sample is one in which the concentration of the mRNA transcript(s) of the gene or genes, or the concentration of the polynucleotides derived from the mRNA transcript(s), is proportional to the transcription level (and therefore expression level) of that gene. Similarly, it is preferred that the hybridization signal intensity be proportional to the amount of hybridized polynucleotide. While it is preferred that the proportionality be relatively strict (e.g., a doubling in transcription rate results in a doubling in mRNA transcript in the sample polynucleotide pool and a doubling in hybridization signal), one of skill will appreciate that the proportionality is more relaxed and even non-linear. Thus, for example, an assay where a 5 fold difference in concentration of the probe mRNA results in a 3 to 6 fold difference in hybridization intensity is sufficient for most purposes. Where more precise quantification is required appropriate controls are run to correct for variations introduced in sample preparation and hybridization as described herein. In addition, serial dilutions of “standard” probe mRNAs are used to prepare calibration curves according to methods well known to those of skill in the art. Of course, where simple detection of the presence or absence of a transcript is desired, no elaborate control or calibration is required.

For example, if a microarray nucleic acid member is not labeled after hybridization, this indicates that the gene comprising that nucleic acid member is not expressed in either sample. If a nucleic acid member is labeled with a single color, it indicates that a labeled gene was expressed only in one sample. The labeling of a nucleic acid member comprising an array with both colors indicates that the gene was expressed in both samples. Even genes expressed once per cell are detected (1 part in 100,000 sensitivity). A 1.4-fold or greater difference in expression intensity in the two samples being compared is indicative of differential expression.

Verification of Differential Expression

The above methods result in the identification, using polynucleotide arrays comprising polynucleotides of known sequences, of nucleic acid molecules that are differentially expressed in an animal subjected to pain. Following the initial identification of such sequences using the microarrays, however, the differential expression is validated using techniques that are well known in the art.

In one embodiment, following identification of a 1.4 fold or greater difference in hybridization intensity in the sample obtained from an animal subjected to pain relative to a naïve animal, reverse transcription PCR (RT-PCR) is performed using primers specific for the hybridizing sequence. For example, given that the identity and sequence of each nucleic acid comprising the polynucleotide array is known, if probe nucleic acid hybridizes at a given position on the array, one of skill in the art can design primers based on the sequence of the nucleic acid known to be at that position, which can then be used to amplify the known sequence from the original nucleic acid sample obtained from the animal. The technique of designing primers for PCR amplification is well known in the art. Oligonucleotide primers and probes are 5 to 100 nucleotides in length, ideally from 17 to 40 nucleotides, although primers and probes of different length are of use. Primers for amplification are preferably about 17-25 nucleotides. Primers useful according to the invention are also designed to have a particular melting temperature (Tm) by the method of melting temperature estimation. Commercial programs, including Oligo™ (MBI, Cascade, Colo.), Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50 and 60° C. Preferably, the Tm of a probe useful according to the invention is 7° C. higher than the Tm of the corresponding amplification primers. It is preferred that, following generation of cDNA by RT-PCR, the cDNA fragment is cloned into an appropriate sequencing vector, such as a PCRII vector (TA cloning kit; Invitrogen). The identity of each cloned fragment is then confirmed by sequencing in both directions. It is expected that the sequence obtained from sequencing would be the same as the known sequence originally spotted on the polynucleotide array.

In one embodiment, following sequence confirmation of the identity of the differentially expressed polynucleotide, the differential expression of the polynucleotide in sensory neurons of an animal subjected to pain relative to a naïve animal is confirmed by Northern analysis. Sequence confirmed cDNAs are used to produce ³²P-labeled cDNA probes using techniques well known in the art (see, for example, Ausubel, supra), or commercially available kits (Prime-It Kit, Stratagene, La Jolla, Calif.). Northern analysis of total RNA obtained from naïve animals and animals subjected to pain is then performed using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at −80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.), wherein at least a 2 fold, preferably a 1.4 fold increase or decrease in the hybridization intensity of the radiolabeled probe obtained from the animal subjected to pain relative to the naïve animal validates the differential expression observed using the polynucleotide microarray.

In an alternate embodiment, the differential expression of polynucleotide sequences, first identified using the polynucleotide microarrays is verified using the Taqman™ (Perkin-Elmer, Foster City, Calif.) techniques, which is performed with a transcript-specific antisense probe. This probe is specific for the PCR product (e.g. a nucleic acid sequence identified using the microarray as being differentially regulated) and is prepared with a quencher and fluorescent reporter probe complexed to the 5′ end of the oligonucleotide. Different fluorescent markers can be attached to different reporters, allowing for measurement of two products in one reaction. When Taq DNA polymerase is activated, it cleaves off the fluorescent reporters by its 5′-to-3′ nucleolytic activity. The reporters, now free of the quenchers, fluoresce. The color change is proportional to the amount of each specific product and is measured by fluorometer; therefore, the amount of each color can be measured and the RT-PCR product can be quantified. The PCR reactions can be performed in 96 well plates so that samples derived from many individuals can be processed and measured simultaneously. The Taqman™ system has the additional advantage of not requiring gel electrophoresis and allows for quantification when used with a standard curve. Quantitative analysis of the mRNA levels for a given gene present in the originally obtained sample from an animal subjected to pain permits a determination of the differential expression of the particular mRNA relative to that obtained from a naïve animal. A fold increase or decrease in expression of a nucleic acid sequence from an animal subjected to pain of at least 2 relative to a naïve animal is indicative of differential expression, and is sufficient to validate the differential expression first identified using the polynucleotide microarray.

In a still further embodiment, the differential expression of a polynucleotide identified using microarray analysis is verified by in situ hybridization. Given that the sequence of each of the nucleic acid molecules on the microarray used to identify differential expression is known, labeled cDNA or antisense RNA probes can be generated using techniques which are known in the art (Ausubel et al., supra). The probes are then hybridized to fixed (e.g., fixed in 4% paraformaldehyde) thin (5-50 μm) tissue sections of, for example, the dorsal root ganglion. Briefly, prior to hybridization, the tissue sections are incubated in acetic anhydride, dehydrated in graded ethanols, and de-lipidated in chloroform. Tissue sections are then hybridized with one or more labeled probes for 24 hours at 45° C. Hybridized probe may be subsequently detected using techniques which are compatible with the label incorporated in the probe. The level of hybridization may be quantitated using any technique known to those of skill in the art. For example, the hybridization signal may be photographed, and the photograph scanned into a computer and the hybridization signal quantitated using software such as NIH Image (NIH, Bethesda, Md.). The measured level of hybridization may then be correlated with the differential expression level measured using the microarray analysis.

In a further embodiment, differential expression of sequences, identified based on the 1.4 fold threshold criteria, described above, can be verified as being differentially expressed if they are differentially expressed by at least 1.2 fold, with a p-value of less than 0.05, in a statistical analysis of triplicate array data points using an appropriate statistical analysis, such as a student's t-test.

Differentially Expressed Polynucleotides

The present invention provides polynucleotides and genes which are differentially expressed in an animal which has been subjected to pain relative to an animal not subjected to pain, wherein the differential expression is determined using the methods described above. Using the above methods a number of polynucleotides have been identified which are differentially expressed in an animal subjected to pain. These polynucleotides and their respective human homologs, as well as the polypeptide molecules encoded thereby are shown in Tables 1, 2, 3, 4, or 5.

Table 1 shows a group of differentially expressed polynucleotides and genes, several of which demonstrate an at least 1.4 fold change in expression in an animal subjected to pain in both axotomy and SNI pain models relative to naïve animals; indicated by the Fold Change of Axotomy/Naïve or SNI/Naïve. Those polynucleotides that are not differentially expressed by at least +/−1.4 fold are not considered to be differentially expressed according to the invention. The polynucleotides of Table 1 have been previously suggested to be involved in the mechanisms of pain and neuronal injury. The present invention, however, distinguishes these polynucleotides by providing a threshold of differential expression which is less than that previously accepted for such analysis.

Table 2 shows polynucleotides of the present invention which have been established as being differentially expressed by at least 1.4 fold in an axotomy, SNI, or inflammation animal pain model, and which have been further analyzed by triplicate analysis as shown in Tables 6 and 7. The polynucleotide sequences shown in Table 2 have been established herein as being differentially expressed by at least 1.2 fold, with a level of statistical significance of p<0.05 as determined by a student's t-test over at least three replicate assays (the replicate assay schemes are shown in Tables 6 and 7), in several animal pain models measured at several post operative time points. The nerve injury pain models represented are the Spinal segmental nerve injury (Chung), Chronic Constriction Injury (CCI) and Spared Nerve Injury (SNI) models at time points 3, 7, 21 and 40 days. The inflammatory model represented is intraplantar Complete Freund's Adjuvant (CFA) injection in to the hind paw at 0.5, 1, and 5 days post injection. The tissue are lumbar DRGs and dorsal horn (i.e two tissues four models, 4 time points (3 for CFA)=30 different pain comparisons each in triplicate each compared against the appropriate control.

Table 3 shows polynucleotide sequences of the present invention which have been established as being differentially expressed by at least 1.4 fold, but which have not attained a statistical significance of p<0.05 according to the triplicate analysis scheme shown in Tables 6 and 7. The polynucleotide sequence shown in Table 3, however, are considered to be “differentially expressed” according to the present invention, despite the fact that the triplicate analysis has not established a significance of p<0.05.

Table 4 shows polynucleotides of the present invention which are upregulated by at least 1.4 fold in a rat inflammation pain model as indicated by either or both of the Intensity Ratio Naïve/SNI or Affymetrix Ratio data column, and which have not been previously suggested to be involved in the cellular response to pain.

Table 5 shows polynucleotides of the present invention which are downregulated by at least 1.4 fold in a rat inflammation pain model as indicated by either or both of the Intensity Ratio Naïve/SNI or Affymetrix Ratio data column, and which have not been previously suggested to be involved in the cellular response to pain. The data in tables 4 and 5 represents an average of the Intensity Ratios and Affymetrix Ratios obtained from inflammation pain models at 3 hours, 6 hours, 12 hours, 24 hours, 48 hours and 5 days following induction of inflammation.

Table 10 shows a subset of the polynucleotides and polypeptides of the Tables 1-5 above, which are differentially expressed in the DH and/or DRG in at least two of the neuropathic pain models selected from spared nerve injury (SNI), chronic constriction injury (CCI), and the CHUNG model. For inclusion in Table 10, the gene had to show two consecutive significant incidents of differential regulation, one of which had to reach the fold threshold. According to the invention, a gene which is differentially expressed in more than one pain model is much more likely to have a general role in pain.

Table 11 shows a subset of the genes of Table 10 which are highly regulated in pain. The genes of Table 10 were selected based on their coordinate regulation in either the DRG or DH and for which, based on the known or deduced function of the gene (e.g., as a transmembrane receptor), have been determined by the invention to have a role in the production or modulation of pain by changing the properties, excitability, structure and/or connectivity of neurons in pain pathways. Accordingly, reference to the genes (polynucleotides or polypeptides) of Tables 1-5 of the invention, necessarily encompasses the genes set forth in Tables 10 and 11. According to the invention, of the genes which are differentially expressed as described herein, the genes of Table 10 is a preferred set of differentially expressed genes useful for screening, treatment, and pain modulation as described below. More preferred for screening assays, treatment, and pain modulation according to the invention, is the set of genes indicated in Table 11.

As indicated in the tables of the invention, the column labeled “% homology” indicates the percent identity between the human and rat (or mouse if the rat sequence is not available) sequences. In some cases, the polynucleotide sequence indicated in Table 2, 3, 4, or 5 is an EST sequence. Accordingly, the column labeled “former identifier” indicates the accession number of the gene sequence having the closest homology, as determined by a BLAST search, to the EST sequence. The column labeled “identifier” in conjunction with the columns labeled “description” and “protein type” indicate the function of the proteins encoded by the polynucleotides of Tables 1, 2, 3, 4, or 5 and specifically indicated in Tables 2, 3, 4, or 5. The column labeled “subcellular localization” indicates the known location of the protein encoded by the polynucleotide sequences noted in the Table in specific compartments in the cell. Accordingly, those proteins which are indicated in the Table as being secreted may be useful, as described below, as protein drugs for modulating the activity of one or more proteins indicated in the table, or for treating pain as described herein. Similarly, proteins which are indicated as being integral membrane proteins may be cell surface receptors, and may be screened against candidate compounds to identify compounds which regulate their activity as described below. The columns labeled “rat gene SEQ ID No.”, “rat protein SEQ ID No.”, “human gene SEQ ID No.”, and “human protein SEQ ID No.” in Tables 2-3 indicates the SEQ ID No. corresponding to the sequence identified by the corresponding accession number.

In addition to the polynucleotides indicated in Tables 1, 2, 3, 4, or 5, the scope of the invention further includes variations, and/or mutations in the polynucleotide sequences, including SNPs and other conservative variants that do not alter the functionality of the encoded polypeptide, including sequences having at least 30% homology with the polynucleotide sequences shown in Tables 1, 2, 3, 4, or 5, but encoding a protein having the equivalent function to the protein encoded by the polynucleotide sequences shown in Tables 1, 2, 3, 4, or 5. The present invention further encompasses the human homologs to the polynucleotide sequences indicated in Tables 1, 2, 3, 4, or 5, and the polypeptide sequences encoded thereby. The invention still further encompasses the polypeptide sequences encoded by the polynucleotide sequences shown in Tables 1, 2, 3, 4, or 5. The Accession no. for the polypeptide sequence is shown in Tables 2, 3, 4, or 5 (the protein accession number is not indicated for Table 1, as all of these genes are known in the art). The present invention also encompasses a variant, domain, epitope, or fragment of the polypeptide molecules indicated in Tables 1, 2, 3, 4, or 5, provided that the variant, domain, epitope, or fragment has an equivalent function to that of the polypeptide indicated in Tables 1, 2, 3, 4, or 5 (i.e., the function for the proteins indicated in Tables).

LENGTHY TABLE REFERENCED HERE
US20070026409A1-20070201-T00001
Please refer to the end of the specification for access instructions.

LENGTHY TABLE REFERENCED HERE
US20070026409A1-20070201-T00002
Please refer to the end of the specification for access instructions.

LENGTHY TABLE REFERENCED HERE
US20070026409A1-20070201-T00003
Please refer to the end of the specification for access instructions.

LENGTHY TABLE REFERENCED HERE
US20070026409A1-20070201-T00004
Please refer to the end of the specification for access instructions.

LENGTHY TABLE REFERENCED HERE
US20070026409A1-20070201-T00005
Please refer to the end of the specification for access instructions.

LENGTHY TABLE REFERENCED HERE
US20070026409A1-20070201-T00006
Please refer to the end of the specification for access instructions.

LENGTHY TABLE REFERENCED HERE
US20070026409A1-20070201-T00007
Please refer to the end of the specification for access instructions.

LENGTHY TABLE REFERENCED HERE
US20070026409A1-20070201-T00008
Please refer to the end of the specification for access instructions.

LENGTHY TABLE REFERENCED HERE
US20070026409A1-20070201-T00009
Please refer to the end of the specification for access instructions.

Vectors and Host Cells

In addition to providing genes which are differentially expressed in animals which have been subjected to pain, the present invention further provides vectors and plasmids useful for directing the expression of differentially expressed genes, or therapeutic nucleic acid constructs, and further provides host cells which express the vectors and plasmids provided herein. Nucleic acid sequences useful for the expression from a vector or plasmid as described below include, but are not limited to any nucleic acid or gene sequence identified as being differentially regulated by the methods described above, and further include therapeutic nucleic acid molecules, such as antisense molecules. The host cell may be any prokaryotic or eukaryotic cell. Ligating the polynucleotide sequence into a gene construct, such as an expression vector, and transforming or transfecting into hosts, either eukaryotic (yeast, avian, insect or mammalian) or prokaryotic (bacterial cells), are standard procedures well known in the art.

Vectors

There is a wide array of vectors known and available in the art that are useful for the expression of differentially expressed nucleic acid molecules according to the invention. The selection of a particular vector clearly depends upon the intended use the polypeptide encoded by the differentially expressed nucleic acid. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the differentially expressed sequences that permit the transcription and translation of the sequence. Sequences that permit the transcription of the linked differentially expressed sequence include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al., 1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) nonmammalian DNA motifs in response to a regulatory agent, and a regulatory sequence that is responsive only to this transcriptional regulator.

Tissue-specific promoters may also be used to advantage in differentially expressed sequence-encoding constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful in the present invention are as follows:

Bowman et al., 1995 Proc. Natl. Acad. Sci. USA 92, 12115-12119 describe a brain-specific transferrin promoter; the synapsin I promoter is neuron specific (Schoch et al., 1996 J. Biol. Chem. 271, 3317-3323); the nestin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); and the potassium channel promoter is high-frequency firing neuron specific (Gan et al., 1996 J. Biol. Chem 271, 5859-5865). Any tissue specific transcriptional regulatory sequence known in the art may be used to advantage with a vector encoding a differentially expressed nucleic acid sequence obtained from an animal subjected to pain.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

a. Plasmid Vectors.

Any plasmid vector that allows expression of a differentially expressed coding sequence of the invention in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of one or more of the differentially expressed polynucleotides of the invention in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Host Cells

Any cell into which a recombinant vector carrying a gene encoding a nucleic acid sequence differentially expressed in an animal subjected to pain may be introduced and wherein the vector is permitted to drive the expression of the peptide encoded by the differentially expressed sequence is useful according to the invention. Any cell in which a differentially expressed molecule of the invention may be expressed and preferably detected is a suitable host, wherein the host cell is preferably a mammalian cell and more preferably a human cell. Vectors suitable for the introduction of differentially expressed nucleic acid sequences to host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells may be primary cultured cells, for example, primary human fibroblasts or keratinocytes, or may be an established cell line, such as NIH3T3, 293T or CHO cells. Further, mammalian cells useful in the present invention may be phenotypically normal or oncogenically transformed. It is assumed that one skilled in the art can readily establish and maintain a chosen host cell type in culture.

Introduction of Vectors to Host Cells.

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™(Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

Following transfection with a vector of the invention, eukaryotic (e.g., human) cells successfully incorporating the construct (intra- or extrachromosomally) may be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening may be used, wherein a negative selection is used to enrich for cells taking up the construct and FACS or fluorescence scanning is used to further enrich for cells expressing differentially expressed polynucleotides or to identify specific clones of cells, respectively. For example, a negative selection with the neomycin analog G418 (Life Technologies, Inc.) may be used to identify cells that have received the vector, and fluorescence scanning may be used to identify those cells or clones of cells that express the vector construct to the greatest extent.

Polynucleotide Arrays Comprising Differentially Expressed Nucleic Acid Sequences

In one embodiment, the present invention provides a pain-specific polynucleotide array comprising nucleic acid sequences that are identified as being differentially expressed in an animal subjected to pain relative to a naïve animal stably associated at discrete predefined regions on a surface. In a preferred embodiment, a pain-specific microarray useful in the present invention comprises one or more polynucleotides shown in Tables 1, 2, 3, 4, or 5. At least one of the polynucleotides comprising a pain-specific array useful in the present invention must be selected from Table 2, 3, 4, or 5. A pain-specific microarray according to the invention preferably comprises between 10 and 20,000 nucleic acid members, and more preferably comprises at least 5000 nucleic acid members. The nucleic acid members are known or novel polynucleotide sequences which have been determined to be differentially expressed as described herein, or any combination thereof. A pain-specific microarray according to the invention may be used, for example, to test therapeutic compounds which may modulate the expression of the sequences comprising the array in an animal subjected to pain. For example, an animal subjected to pain may be treated with a potentially therapeutic compound as described below. Total RNA may then be extracted from, for example, primary sensory neurons, prepared according to the methods described above, and hybridized to the pain-specific microarray. The level of hybridization of samples to the pain-specific microarray may be compared to the level of hybridization of a nucleic acid sample obtained from an animal subjected to pain, but not administered the therapeutic compound. The pain-specific microarray may also be used, for example, to test the ability of an antisense nucleic acid to hybridize to the differentially expressed nucleic acid molecules comprising the pain-specific microarray. The antisense molecules may then be used to inhibit the expression of, for example, nucleic acid sequences which have been identified, using the above methods, as being upregulated (i.e., by at least 1.4 fold) in an animal subjected to pain.

The invention also provides for a pain-specific microarray comprising nucleic acids sequences which have been identified and verified as being differentially expressed in an animal subjected to pain, wherein the sequences stably associated with the array are obtained from at least two different species of animal. In a preferred embodiment, a pain-specific microarray useful in the present invention comprises at least one polynucleotide shown in Table 2, 3, 4, or 5, and may optionally further comprise one or more of the polynucleotides shown in Table 1. Such arrays may also be used for prognostic methods to monitor an animal's response to therapy. In one embodiment, the above pain-specific microarrays are used to identify a therapeutic agent that changes (e.g., increases or decreases) the level of expression of at least one polynucleotide sequence that is differentially expressed (i.e., by at least 1.4 fold, or at least 1.2 fold in combination with a p-value of less than 0.05 in triplicate analysis) in sensory neurons in an animal subjected to pain.

The nucleic acid samples that are hybridized to and analyzed with a pain-specific microarray of the invention are preferably derived from sensory neurons of an animal subjected to pain (or from a naïve control animal). More preferably, the nucleic acid samples are obtained from primary sensory neurons of the dorsal root ganglion. A limitation for this procedure lies in the amount of RNA available for use as a probe nucleic acid sample. Preferably, at least 1 microgram of total RNA is obtained for use according to this invention.

Construction of a Pain-Specific Microarray

An aspect of the present invention incorporates the previously identified differentially regulated nucleic acid sequences into a pain-specific polynucleotide microarray. In the present methods, an array of nucleic acid members stably associated with the surface of a substantially planar solid support is contacted with a sample comprising probe polynucleotides obtained from an animal subjected to pain, or from a naïve animal under hybridization conditions sufficient to produce a hybridization pattern of complementary nucleic acid members/probe complexes.

The nucleic acid members may be produced using established techniques such as polymerase chain reaction (PCR) and reverse transcription (RT). For example, once a nucleic acid sequence has been identified as being differentially expressed in an animal subjected to pain, the sequence may be amplified from the originally obtained RNA sample by RT-PCR, wherein the amplified product may be used to construct a pain-specific microarray. These methods are similar to those currently known in the art (see e.g. PCR Strategies, Michael A. Innis (Editor), et al. (1995) and PCR: Introduction to Biotechniques Series, C. R. Newton, A. Graham (1997)). Amplified polynucleotides are purified by methods well known in the art (e.g., column purification or alcohol precipitation). A polynucleotide is considered pure when it has been isolated so as to be substantially free of primers and incomplete products produced during the synthesis of the desired polynucleotide. Preferably, a purified polynucleotide will also be substantially free of contaminants which may hinder or otherwise mask the binding activity of the molecule.

A pain-specific microarray according to the invention comprises a plurality of unique polynucleotides attached to one surface of a solid support at a density exceeding 20 different polynucleotides/cm², wherein each of the polynucleotides is attached to the surface of the solid support in a non-identical preselected region. Each associated sample on the array comprises a polynucleotide composition, of known identity, usually of known sequence, as described in greater detail below. Any conceivable substrate may be employed in the invention. In one embodiment, the polynucleotide attached to the surface of the solid support is DNA. In a preferred embodiment, the polynucleotide attached to the surface of the solid support is cDNA or RNA. In another preferred embodiment, the polynucleotide attached to the surface of the solid support is cDNA synthesized by polymerase chain reaction (PCR). Preferably, a nucleic acid member comprising an array, according to the invention, is at least 25 nucleotides in length. In one embodiment, a nucleic acid member comprising an array is at least 150 nucleotides in length. Preferably, a nucleic acid member comprising an array is less than 1000 nucleotides in length. More preferably, a nucleic acid member comprising an array is less than 500 nucleotides in length. In one embodiment, an array comprises at least 10 different polynucleotides attached to one surface of the solid support. In another embodiment, the array comprises at least 100 different polynucleotides attached to one surface of the solid support. In yet another embodiment, the array comprises at least 10000 different polynucleotides attached to one surface of the solid support.

In the arrays of the invention, the polynucleotide compositions are stably associated with the surface of a solid support, wherein the support may be a flexible or rigid solid support. By “stably associated” is meant that each nucleic acid member maintains a unique position relative to the solid support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the polynucleotides and a functional group present on the surface of the rigid support (e.g., —OH), where the functional group may be naturally occurring or present as a member of an introduced linking group, as described in greater detail below

The amount of differentially expressed polynucleotide present in each composition will be sufficient to provide for adequate hybridization and detection of probe polynucleotide sequences during the assay in which the array is employed. Generally, the amount of each nucleic acid member stably associated with the solid support of the array is at least about 0.1 ng, preferably at least about 0.5 ng and more preferably at least about 1 ng, where the amount may be as high as 1000 ng or higher, but will usually not exceed about 20 ng. Where the nucleic acid member is “spotted” onto the solid support in a spot comprising an overall circular dimension, the diameter of the “spot” will generally range from about 10 to 5,000 μm, usually from about 20 to 2,000 μm and more usually from about 50 to 1000 μm.

Control nucleic acid members may be present on the array including nucleic acid members comprising oligonucleotides or polynucleotides corresponding to genomic DNA, housekeeping genes, vector sequence, plant nucleic acid sequence, negative and positive control genes, and the like. Control nucleic acid members are calibrating or control genes whose function is not to tell whether a particular “key” gene of interest is expressed, but rather to provide other useful information, such as background or basal level of expression.

Other control polynucleotides are spotted on the array and used as probe expression control polynucleotides and mismatch control nucleotides to monitor non-specific binding or cross-hybridization to a polynucleotide in the sample other than the target to which the probe is directed. Mismatch probes thus indicate whether a hybridization is specific or not. For example, if the target is present, the perfectly matched probes should be consistently brighter than the mismatched probes.

Solid Substrate

An array according to the invention comprises either a flexible or rigid substrate. A flexible substrate is capable of being bent, folded or similarly manipulated without breakage. Examples of solid materials which are flexible solid supports with respect to the present invention include membranes, e.g., nylon, flexible plastic films, and the like. By “rigid” is meant that the support is solid and does not readily bend, i.e., the support is not flexible. As such, the rigid substrates of the subject arrays are sufficient to provide physical support and structure to the associated polynucleotides present thereon under the assay conditions in which the array is employed, particularly under high throughput handling conditions.

The substrate may be biological, non-biological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat or planar but may take on a variety of alternative surface configurations. The substrate may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure.

In a preferred embodiment the substrate is flat glass or single-crystal silicon. According to some embodiments, the surface of the substrate is etched using well known techniques to provide for desired surface features. For example, by way of the formation of trenches, v-grooves, mesa structures, or the like, the synthesis regions may be more closely placed within the focus point of impinging light, be provided with reflective “mirror” structures for maximization of light collection from fluorescent sources, etc.

Surfaces on the solid substrate will usually, though not always, be composed of the same material as the substrate. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. In some embodiments the surface may provide for the use of caged binding members which are attached firmly to the surface of the substrate. Preferably, the surface will contain reactive groups, which are carboxyl, amino, hydroxyl, or the like. Most preferably, the surface will be optically transparent and will have surface Si—OH functionalities, such as are found on silica surfaces.

The surface of the substrate is preferably provided with a layer of linker molecules, although it will be understood that the linker molecules are not required elements of the invention. The linker molecules are preferably of sufficient length to permit polynucleotides of the invention and on a substrate to hybridize to other polynucleotide molecules and to interact freely with molecules exposed to the substrate.

Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent. In a preferred embodiment, the substrate is a poly-lysine coated slide or Gamma amino propyl silane-coated Corning Microarray Technolgy-GAPS.

Any solid support to which a nucleic acid member may be attached may be used in the invention. Examples of suitable solid support materials include, but are not limited to, silicates such as glass and silica gel, cellulose and nitrocellulose papers, nylon, polystyrene, polymethacrylate, latex, rubber, and fluorocarbon resins such as TEFLON™.

The solid support material may be used in a wide variety of shapes including, but not limited to slides and beads. Slides provide several functional advantages and thus are a preferred form of solid support. Due to their flat surface, probe and hybridization reagents are minimized using glass slides. Slides also enable the targeted application of reagents, are easy to keep at a constant temperature, are easy to wash and facilitate the direct visualization of RNA and/or DNA immobilized on the solid support. Removal of RNA and/or DNA immobilized on the solid support is also facilitated using slides.

The particular material selected as the solid support is not essential to the invention, as long as it provides the described function. Normally, those who make or use the invention will select the best commercially available material based upon the economics of cost and availability, the expected application requirements of the final product, and the demands of the overall manufacturing process.

Spotting Method

The invention provides for arrays wherein each nucleic acid member comprising the array is spotted onto a solid support.

Preferably, spotting is carried out as follows. PCR products (˜40 ul) of cDNA clones obtained from animals subjected to pain, in the same 96-well tubes used for amplification, are precipitated with 4 ul (1/10 volume) of 3M sodium acetate (pH 5.2) and 100 ul (2.5 volumes) of ethanol and stored overnight at −20° C. They are then centrifuged at 3,300 rpm at 4° C. for 1 hour. The obtained pellets are washed with 50 ul ice-cold 70% ethanol and centrifuged again for 30 minutes. The pellets are then air-dried and resuspended well in 20 ul 3×SSC overnight. The samples are then spotted, either singly or in duplicate, onto polylysine-coated slides (Sigma Cat. No. P0425) using a robotic GMS 417 arrayer (Affymetrix, CA).

The boundaries of the spots on the microarray are marked with a diamond scriber (note that the spots become invisible after post-processing). The arrays are rehydrated by suspending the slides over a dish of warm particle free ddH₂O for approximately one minute (the spots will swell slightly but will not run into each other) and snap-dried on a 70-80° C. inverted heating block for 3 seconds. Nucleic acid is then UV crosslinked to the slide (Stratagene, Stratalinker, 65 mJ—set display to “650” which is 650×100 uJ). The arrays are placed in a slide rack. An empty slide chamber is prepared and filled with the following solution: 3.0 grams of succinic anhydride (Aldrich) was dissolved in 189 ml of 1-methyl-2-pyrrolidinone (rapid addition of reagent is crucial); immediately after the last flake of succinic anhydride is dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are then stored in the slide box at room temperature until use.

Numerous methods may be used for attachment of the nucleic acid members of the invention to the substrate (a process referred as spotting). For example, polynucleotides are attached using the techniques of, for example U.S. Pat. No. 5,807,522, which is incorporated herein by reference for teaching methods of polymer attachment.

Alternatively, spotting may be carried out using contact printing technology.

Kits

The invention provides for kits for performing expression assays using the pain-specific arrays of the present invention. Such kits according to the present invention will at least comprise the pain-specific arrays of the invention having associated differentially expressed nucleic acid members and packaging means therefore. The kits may further comprise one or more additional reagents employed in the various methods, such as: 1) primers for generating test polynucleotides; 2) dNTPs and/or rNTPs (either premixed or separate), optionally with one or more uniquely labeled dNTPs and/or rNTPs (e.g., biotinylated or Cy3 or Cy5 tagged dNTPs); 3) post synthesis labeling reagents, such as chemically active derivatives of fluorescent dyes; 4) enzymes, such as reverse transcriptases, DNA polymerases, and the like; 5) various buffer mediums, e.g., hybridization and washing buffers; 6) labeled probe purification reagents and components, like spin columns, etc.; and 7) signal generation and detection reagents, e.g., streptavidin-alkaline phosphatase conjugate, chemifluorescent or chemiluminescent substrate, and the like.

Therapeutic Agents and Screening Methods

The present invention provides a number of potentially therapeutic compounds which may be used to modulate the expression of genes which are differentially expressed in an animal subjected to pain, or which may be used to modulate the activity of a protein encoded by a differentially expressed polynucleotide sequence of the invention, or which may be used to modulate pain in an animal. Such therapeutic agents include, but are not limited to a chemical compound, a protein, an antibody, RNAi, and an antisense nucleic acid. In a further aspect, the invention provides a method for screening potentially therapeutic agents for the ability to modulate the expression of genes which are differentially expressed in an animal subjected to pain, and further provides pharmaceutical formulations comprising the therapeutic agents. In a still further embodiment, the present invention provides a method of screening potentially therapeutic agents for the ability to modulate the activity of one or more polypeptides encoded by one or more of the polynucleotide sequences indicated in Tables 1, 2, 3, 4, or 5, or Tables 10 and/or 11.

Therapeutic Agents

A therapeutic agent, useful in the present invention, changes (e.g., increases or decreases) the level of expression of at least one polynucleotide sequence that is differentially expressed in an animal subjected to pain. Preferably, a therapeutic agent causes a change in the level of expression of a polynucleotide sequence, that is, to increase or decrease the expression of a polynucleotide sequence that is differentially expressed in an animal subjected to pain, wherein the change results in the differentially expressed sequence being no longer differentially expressed by at least 1.4 fold (or differentially expressed by 1.2 fold in combination with a statistical significance of p<0.05 in at least three replicate assays) relative to the expression of the same sequence in a naïve animal.

In another embodiment, a therapeutic agent according to the invention can modulate the activity of one or more of the polypeptides specifically indicated in Tables 1, 2, 3, 4, or 5, or encoded by one or more of the polynucleotide sequences of Tables 1, 2, 3, 4, or 5.

In another embodiment, a therapeutic agent according to the invention can ameliorate at least one of the symptoms and/or physiological changes associated with pain including, but not limited to mechanical allodynia and hyperalgesia, and temperature allodynia and hyperalgesia.

The candidate therapeutic agent may be a synthetic compound, or a mixture of compounds, or may be a natural product (e.g. a plant extract or culture supernatant). According to the invention, a therapeutic agent or compound can be a candidate or test compound. Similarly, according to the invention, a candidate or test compound can be a therapeutic agent.

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds [Lam, (1997)]. Examples of methods for the synthesis of molecular libraries can be found in the art. Libraries of compounds may be presented in solution or on beads, bacteria, spores, plasmids or phage.

Candidate therapeutic agents or compounds from large libraries of synthetic or natural compounds may be screened as described below. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, NH), and Microsource (New Milford, Conn.). A rare chemical library is available from Aldrich (Milwaukee, Wis.). Combinatorial libraries are available and are prepared. Alternatively, libraries of natural compounds in the form of bacterial, fungal, plant and animal extracts are available from e.g., Pan Laboratories (Bothell, Wash.) or MycoSearch (NC), or are readily produced by methods well known in the art. Additionally, natural and synthetically produced libraries and compounds are readily modified through conventional chemical, physical, and biochemical means.

Small Molecules

Useful compounds may be found within numerous chemical classes. Useful compounds may be organic compounds, or small organic compounds. Small organic compounds, or “small molecules” have a molecular weight of more than 50 yet less than about 2,500 daltons, preferably less than about 750, more preferably less than about 350 daltons. Exemplary classes include heterocycles, peptides, saccharides, steroids, and the like. Small molecules can be nucleic acids, peptides, polypeptides, peptidomimetics, carbohydrates, lipids or other organic (carbon-containing) or inorganic molecules. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

Antisense Therapy

In one embodiment, a therapeutic agent, according to the invention, can be a differentially expressed nucleic acid or a sequence complementary thereto, useful in antisense therapy. The antisense sequence of a polynucletoide which is differentially expressed in an animal subjected to pain may be determined using the either the sequence indicated by accession number in tables 4-5, or the sequence of the rat and/or human differentially expressed sequences shown in Table 2-3 as set forth in the corresponding SEQ ID No. As used herein, antisense therapy refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA identified as being differentially expressed in an animal subjected to pain. The construction and use of expression plasmids is described above and may be adapted by one of skill in the art to include expression plasmids or vectors comprising anitsense oligonucleotides. Alternatively, the antisense construct is an oligonucleotide probe which is generated ex vivo and which, when introduced into the cell, causes inhibition of expression by hybridizing with the mRNA and/or genomic sequences of a differentially expressed nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA (i.e., differentially expressed mRNA). The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the differentially expressed mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less than about 100 and more preferably less than about 50, 25, 17 or 10 nucleotides in length.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/098 10, published Dec. 15, 1988) or the blood-brain barrier (see, e.g., PCT Publication No. WO 89/10 134, published Apr. 25, 1988), hybridization-triggered cleavage agents (See, e.g., Krol et al., 1988, BioTechniques 6:958-976), or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Peny-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual n-units, the strands run parallel to each other (Gautier et al, 1987, Nucl. Acids Res. 15:6625-6641). The oligonucleotide is a 2′-O-methylribonucleotide (Inoue et al., 1987, Nucl. Acids Res. 15:6131-12148), or a chimeric RNA-DNA analogue (Jnoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.) based on the known sequence of the differentially expressed nucleic acid sequences. As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to a coding region sequence can be used, those complementary to the transcribed untranslated region and to the region comprising the initiating methionine are most preferred.

The antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in an animal will result in the transcription of sufficient amounts of single stranded RNAs that will form complementary base pairs with the endogenous transcripts and thereby prevent translation of the target mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art, combined with those described above. Vectors can be plasmid, viral, or others known in the art for replication and expression in mammalian cells. Expression of the sequence encoding the antisense RNA can be by any promoter known in the art to act in animal, preferably mammalian cells. Such promoters can be inducible or constitutive. Such promoters include but are not limited to: the SV40 early promoter region (Bemoist and Chambon, 1981, Nature 290:304-3 10), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site; e.g., the spinal cord, or dorsal root ganglion. Alternatively, viral vectors can be used which selectively infect the desired tissue (e.g., for brain, herpesvirus vectors may be used), in which case administration may be accomplished by another route (e.g., systemically).

Ribozymes

In another aspect of the invention, ribozyme molecules designed to catalytically cleave target mRNA transcripts can be used to prevent translation of target mRNA and expression of a target protein (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. Ribozymes, useful in the present invention may be designed based on the known sequence of the nucleic acid sequence identified as being differentially expressed in an animal subjected to pain as described above. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Antisense RNA, DNA, and ribozyme molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. The sequences of the antisense and ribozyme molecules will be based on the known sequence of the differentially expressed nucleic acid molecules. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

RNAi Therapy

In another embodiment, a therapeutic agent according to the invention can be a double stranded RNAi molecule that is specifically targeted to one or more of the polynucleotide sequences which are differentially expressed in an animal subjected to pain relative to an animal that is not subjected to pain (see Tables 1, 2, 3, 4, or 5). As used herein, RNAi or RNA interference refers to the gene-specific, double stranded RNA (dsRNA) mediated, post-transcriptional silencing of gene expression as described in the review by Hannon, G., (2002) Nature 418, 244-250, which is herein incorporated in its entirety. Current experimental evidence indicates that RNAis specific for a target RNA are recognized and processed into 21 and 23 nucleotide small interfering RNAs (siRNAs) by the Dicer RNase III endonuclease. SiRNAs are then incorporated into a RNA induced silencing complex (RISC) which becomes activated by unwinding of the duplex siRNA. Activated RISC complexes then promote RNA degradation and translation inhibition of the target RNA.

In mammals, RNAi therapy, according to the invention, refers to gene-specific suppression that can be achieved by generating siRNA (Elbashir, S. M. et al. (2001) Nature (London) 411, 494-498). In vitro synthesized siRNAs can be prepared by any method known in the art for the synthesis of RNA molecules. These include techniques for chemically synthesizing oligoribonucleotides that are well known in the art, for example, solid phase phosphoramidite chemical synthesis. The sequences of the siRNA molecules are based on the known sequence of the differentially expressed nucleic acid molecules. Alternatively, siRNA molecules can be generated by the T7 or SP6 polymerase promoter driven in vitro transcription of DNA sequences encoding the siRNA molecule. In vitro synthesized siRNAs can be delivered to cells either by direct injection of in vitro synthesized siRNAs into the tissue site. Alternatively, modified siRNAs, designed to target the desired cells (via linkage to peptides or antibodies that specifically bind to cell surface receptors or antigens), can be administered systemically.

In a preferred embodiment, the siRNAs of the invention are delivered to a target cell as an expression plasmid under the control of a RNA polymerase II or III promoter. When transcribed in the cell, siRNA is generated which is complementary to a cellular mRNA identified as being differentially expressed in an animal subjected to pain. The construction and use of expression plasmids is described above and may be adapted by one of skill in the art to include siRNA expression plasmids. Such vectors can be constructed by recombinant DNA technology methods standard in the art, combined with those described above. Vectors can be plasmid, viral, or others known in the art for replication and expression in mammalian cells. Expression of the sequence encoding the siRNA can be by any promoter known in the art to act in an animal, preferably mammalian cells. Such promoters can be inducible or constitutive. Such promoters include but are not limited to: the SV40 early promoter region (Bernoist and Chambon, 1981, Nature 290:304-3 10), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc as well as neural specific promoters, for example the nestin promoter. Any plasmid, cosmid, YAC or viral vector can be used to prepare the recombinant DNA construct which can be introduced directly into the tissue site; e.g., the spinal cord, or dorsal root ganglion. Alternatively, viral vectors can be used which selectively infect the desired tissue (e.g., for brain, herpes virus vectors may be used), in which case administration may be accomplished by another route (e.g., systemically).

In a preferred embodiment, the siRNA expression vectors of the invention are synthesized from a DNA template under the control of an RNA polymerase III (Pol III) promoter in transfected cells or transgenic animals (see below). Pol III directs the synthesis of small, noncoding transcripts whose 3′ ends are defined by termination within a stretch of 4-5 thymidines (Ts) (Sui et al. PNAS (2002) vol. 99, 5515-5520). Addition of 3′ overhangs contributes to the activity of siRNA synthesized in vitro (Elbashir, S. M et al. (2001) Genes Dev. 15, 188-200). Transfection of such a construct into target cells results in the transcription of sufficient amounts of siRNAs to base pair with the endogenous transcripts, promote its degradation and thereby prevent translation of the target mRNA. The vector can remain episomal or become chromosomally integrated. Alternatively the construct may be incorporated into a viral vector such as herpes virus vectors as described supra.

An example of mouse U6 pol III transcribed siRNA expression plasmid is shown below where the 21 nucleotide sequence is specific for one or more of the differentially expressed sequences shown in Tables 1, 2, 3, 4, or 5 (see Sui et al. PNAS (2002) vol. 99, 5515-5520):

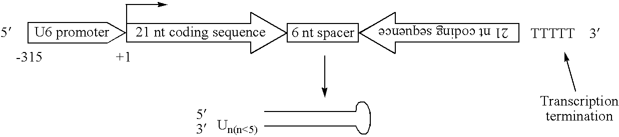

Supplemental Therapy

The differentially expressed nucleic acid sequences described herein may exhibit either increased or decreased expression. The antisense methods described above are directed primarily at inhibiting the expression of a differentially overexpressed sequence. Alternatively, in the situation where differential expression is manifested in a decrease in sequence expression, the underexpressed sequence may be supplied to the animal in an expression vector as described above. If for example, through the process of identifying and verifying the differential expression of nucleic acid sequences obtained from an animal subjected to pain, a sequence is identified which is expressed at a level at least 1.2 fold less than in a naïve animal in at least three replicate analyses with a significance of p<0.05 (or, alternatively, at least 1.4 fold less), the sequence may be cloned into a suitable expression vector for expression of the sequence in the animal subjected to pain. Either viral or non-viral gene delivery methods may be used to introduce the construct into the animal cells as described above. Briefly, the deficient sequence may be cloned into any expression vector known in the art which is compatible with the animal cell into which it is intended to be introduced, and which is capable of supporting expression of the recombinant sequence. The vector used may be chosen to replicate episomaly or may integrate in the cell chromosome, provided that either mode of replication permits the expression of the deficient nucleic acid sequence. Further, any promoter sequence which is sufficient to direct expression of the recombinant sequence may be used in the vector to direct expression of the sequence. In a preferred embodiment, the promoter is constitutively active in the animal, given that the goal is to attain a level of gene expression sufficient to replace the deficiently expressed sequence. In a further preferred embodiment, the promoter is a neuron-specific promoter. Vectors comprising the deficient sequence may be introduced into cells of the animal subjected to pain using any technique known to those of skill in the art including, but not limited to microinjection and viral delivery.

Similarly, those proteins which are encoded by polynucleotide sequences which are differentially expressed as indicated in Tables 1, 2, 3, 4, or 5, and which are also indicated in the column labeled “subcellular localization” (i.e., in Table 2) as being a secreted protein, may be screened for their ability to modulate the activity of one or more of the proteins indicated in Tables 1, 2, 3, 4, or 5, or screened for their ability to modulate pain in an animal.

Once a therapeutic gene is defined, whether it be an antisense molecule, ribozyme, or supplemental sequence, the gene sequence is subcloned into a vector suitable for the purpose of gene therapy. Murine leukemia virus (MLV)-based retroviral vectors are one of the most widely used gene delivery vehicles in gene therapy clinical trials and have been employed in almost 70% of approved protocols (Ali, M. et al., Gene Ther., 1:367-384, 1994; Marshall, E., Science, 269:1050-1055, 1995). Other useful vectors are also known in the art (e.g., Carter and Samulski, 2000, Int. J. Mol. Med. 6:17-27; Lever et al., 1999, Biochem. Soc. Trans. 27: 841-7). Methods for gene therapy of human diseases are described in U.S. Pat. Nos. 6,190,907; 6,187,305; 6,140,087; and 6,129,705.

Screening Assays

Protein Activity Regulators

Regulators as used herein, refer to compounds that affect the activity of a “differentially expressed protein” in vivo and/or in vitro. As used herein, the term “differentially expressed protein (or polypeptide)” will refer to the proteins of Table 1, 2, 3, 4, or 5 that are encoded by sequences that are differentially expressed in pain. Regulators can be agonists and antagonists of a differentially expressed polypeptide and can be compounds that exert their effect on the differentially expressed protein activity via the enzymatic activity, expression, post-translational modifications or by other means. Agonists of a differentially expressed protein are molecules which, when bound to a differentially expressed protein, increase or prolong the activity of a differentially expressed protein. Agonists of a differentially expressed protein include proteins, nucleic acids, carbohydrates, small molecules, or any other molecule which activate a differentially expressed protein. Antagonists of a differentially expressed protein are molecules which, when bound to a differentially expressed protein, decrease the amount or the duration of the activity of a differentially expressed protein. Antagonists include proteins, nucleic acids, carbohydrates, antibodies, small molecules, or any other molecule which decrease the activity of a “differentially expressed protein”. The activity of a differentially expressed protein, useful in the present invention is indicated in Table 2, 3, 4, or 5 either directly in columns labeled “identifier”, “description” and/or “protein type”, or may be inferred from the information provided in the column labeled “subcellular localization” (Table 2). For example, if a protein is localized to the cell membrane, then one of skill in the art would be able to determine that the activity of such a protein would be that of a receptor, for example, or an ion channel, and screen candidate compounds against this protein activity accordingly.

The term “modulate”, as it appears herein, refers to a change in the activity of a differentially expressed protein. For example, modulation may cause an increase or a decrease in enzymatic activity, binding characteristics, or any other biological, functional, or immunological properties of a differentially expressed protein.

As used herein, the terms “specific binding” or “specifically binding” refer to that interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A” the presence of a polypeptide containing the epitope A, or the presence of free unlabeled A, in a reaction containing free labeled A and the antibody will reduce the amount of labeled A that binds to the antibody.

The invention provides methods (also referred to herein as “screening assays”) for identifying compounds which can be used for the treatment of pain. The methods entail the identification of candidate or test compounds or agents (e.g., peptides, peptidomimetics, small molecules or other molecules) which bind to a differentially expressed protein and/or have a stimulatory or inhibitory effect on the biological activity of a differentially expressed protein or its expression and then determining which of these compounds have an effect on pain symptoms in an in vivo assay.

Candidate or test compounds or agents which bind to a differentially expressed protein and/or have a stimulatory or inhibitory effect on the activity or the expression of a differentially expressed protein are identified either in assays that employ cells which express a differentially expressed protein (cell-based assays) or in assays with an isolated differentially expressed protein (cell-free assays). The various assays can employ a variety of variants of a differentially expressed protein (e.g., full-length differentially expressed protein, a biologically active fragment of a differentially expressed protein, or a fusion protein which includes all or a portion of a differentially expressed protein). Moreover, a differentially expressed protein can be derived from any suitable mammalian species (e.g., human differentially expressed protein, rat differentially expressed protein or murine differentially expressed protein). The assay can be a binding assay entailing direct or indirect measurement of the binding of a test compound or a known differentially expressed protein ligand to a differentially expressed protein. The assay can also be an activity assay entailing direct or indirect measurement of the activity of a differentially expressed protein. The assay can also be an expression assay entailing direct or indirect measurement of the expression of a differentially expressed protein mRNA or a differentially expressed protein. The various screening assays are combined with an in vivo assay entailing measuring the effect of the test compound on the pain symptoms.

In one embodiment, the invention provides assays for screening candidate or test compounds which bind to or modulate the activity of a membrane-bound (cell surface expressed) form of the differentially expressed protein. Such assays can employ the full-length differentially expressed protein, a biologically active fragment of the differentially expressed protein, or a fusion protein which includes all or a portion of the differentially expressed protein. As described in greater detail below, the test compound can be obtained by any suitable means, e.g., from conventional compound libraries. Determining the ability of the test compound to bind to a membrane-bound form of the differentially expressed protein can be accomplished, for example, by coupling the test compound with a radioisotope or enzymatic label such that binding of the test compound to the differentially expressed protein-expressing cell can be measured by detecting the labelled compound in a complex. For example, the test compound can be labelled with ¹²⁵I, ³⁵S, ¹⁴C, or ³H, either directly or indirectly, and the radioisotope detected by direct counting of radioemmission or by scintillation counting. Alternatively, the test compound can be enzymatically labelled with, for example, horseradish peroxidase, alkaline phosphatase, or luciferase, and the enzymatic label detected by determination of conversion of an appropriate substrate to product.

In a competitive binding format, the assay comprises contacting the differentially expressed protein-expressing cell with a known compound which binds to the differentially expressed protein to form an assay mixture, contacting the assay mixture with a test compound, and determining the ability of the test compound to interact with the differentially expressed protein-expressing cell, wherein determining the ability of the test compound to interact with the differentially expressed protein-expressing cell comprises determining the ability of the test compound to preferentially bind the differentially expressed protein expressing cell as compared to the known compound.

In another embodiment, the assay is a cell-based assay comprising contacting a cell expressing a membrane-bound form of the differentially expressed protein (e.g., full-length differentially expressed protein, a biologically active fragment of the differentially expressed protein, or a fusion protein which includes all or a portion of the differentially expressed protein) expressed on the cell surface with a test compound and determining the ability of the test compound to modulate (e.g., stimulate or inhibit) the activity of the membrane-bound form of the differentially expressed protein. Determining the ability of the test compound to modulate the activity of the membrane-bound form of the differentially expressed protein can be accomplished by any method suitable for measuring the activity of the differentially expressed protein, e.g., any method suitable for measuring the activity of a G-protein coupled receptor or other seven-transmembrane receptor (described in greater detail below). The activity of a seven-transmembrane receptor can be measured in a number of ways, not all of which are suitable for any given receptor. Among the measures of activity are: alteration in intracellular Ca2+ concentration, activation of phospholipase C, alteration in intracellular inositol triphosphate (IP3) concentration, alteration in intracellular diacylglycerol (DAG) concentration, and alteration in intracellular adenosine cyclic 3′,5′-monophosphate (cAMP) concentration.

The present invention includes biochemical, cell free assays that allow the identification of inhibitors and agonists of phosphodiesterases (PDEs) suitable as lead structures for pharmacological drug development. Such assays involve contacting a form of a differentially expressed protein (e.g., full-length differentially expressed protein, a biologically active fragment of a differentially expressed protein, or a fusion protein comprising all or a portion of a differentially expressed protein) with a test compound and determining the ability of the test compound to act as an antagonist (preferably) or an agonist of the enzymatic activity of a differentially expressed protein. In one embodiment, the assay includes monitoring the PDE activity of a differentially expressed protein by measuring the conversion of either cAMP or cGMP to its nucleoside monophosphate after contacting a differentially expressed protein with a test compound.

For example, cAMP and cGMP levels can be measured by the use of the tritium containing compounds 3HcAMP and 3HcGMP as described in [Hansen, R. S., and Beavo, J. A., PNAS USA1982;79: 2788-92]. To screen a compound pool comprised of a large number of compounds, the microtiter plate-based scintillation proximity assay (SPA) as described in [Bardelle, C. et al. (1999) Anal. Biochem. 275: 148-155] can be applied.

Alternatively, the phosphodiesterase activity of the recombinant protein can be assayed using a commercially available SPA kit (Amersham Pharmacia). The PDE enzyme hydrolyzes cyclic nucleotides, e.g. cAMP and cGMP to their linear counterparts. The SPA assay utilizes the tritiated cyclic nucleotides [3H]cAMP or [3H]cGMP, and is based upon the selective interaction of the tritiated non cyclic product with the SPA beads whereas the cyclic substrates are not effectively binding. Radiolabelled product bound to the scintillation beads generates light that can be analyzed in a scintillation counter.

The cell-free assays of the present invention are amenable to use of either a membrane-bound form of the differentially expressed protein or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include, but are not limited to, non-ionic detergents such as n-octylglucoside, n-dodecylglucoside, n-dodecylmaltoside, octanoyl-N-methyl-glucamide, decanoyl-N-methylglucamide, Triton X-100, Triton X-114, Thesit, Iso-tri-decy-poly-(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one embodiment, the invention provides assays for screening candidate or test compounds which bind to or modulate the activity of a differentially expressed protein. Such assays can employ full-length differentially expressed protein, a biologically active fragment of a differentially expressed protein, or a fusion protein which includes all or a portion of a differentially expressed protein. As described in greater detail below, the test compound can be obtained by any suitable means, e.g., from conventional compound libraries.

Determining the ability of the test compound to modulate the activity of a differentially expressed protein can be accomplished, for example, by determining the ability of a differentially expressed protein to bind to or interact with a target molecule. The target molecule can be a molecule with which a differentially expressed protein binds or interacts with in nature. The target molecule can be a component of a signal transduction pathway which facilitates transduction of an extracellular signal. The target differentially expressed protein molecule can be, for example, a second intracellular protein which has catalytic activity or a protein which facilitates the association of downstream signaling molecules with a differentially expressed protein.

Determining the ability of a differentially expressed protein to bind to or interact with a target molecule can be accomplished by one of the methods described above for determining direct binding. In one embodiment, determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca2+, diacylglycerol, IP3, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response.

In various embodiments of the above assay methods of the present invention, it may be desirable to immobilize a differentially expressed protein (or a differentially expressed protein target molecule) to facilitate separation of complexed from uncomplexed forms of one or both of the proteins, as well as to accommodate automation of the assay. Binding of a test compound to a differentially expressed protein, or interaction of a differentially expressed protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-5-transferase (GST) fusion proteins or glutathione-5-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a differentially expressed protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of a differentially expressed protein can be determined using standard techniques.

Other techniques for immobilizing proteins on matrices can also be used in the screening assays of the invention. For example, either a differentially expressed protein or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated plates (Pierce Chemical). Alternatively, antibodies reactive with a differentially expressed protein or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immuno-detection of complexes using antibodies reactive with a differentially expressed protein or target molecule, as well as enzyme-linked assays which rely on detecting an enzymatic activity associated with a differentially expressed protein or target molecule.

Another technique for drug screening which may be used provides for high throughput screening of compounds having suitable binding affinity to the protein of interest as described in published PCT application WO84/03564. In this method, large numbers of different small test compounds are synthesized on a solid substrate, such as plastic pins or some other surface. The test compounds are reacted with a differentially expressed protein, or fragments thereof, and washed. Bound differentially expressed protein is then detected by methods well known in the art. Purified differentially expressed protein can also be coated directly onto plates for use in the afore-mentioned drug screening techniques. Alternatively, non-neutralizing antibodies can be used to capture the peptide and immobilize it on a solid support.

In another embodiment, one may use competitive drug screening assays in which neutralizing antibodies capable of binding differentially expressed protein specifically compete with a test compound for binding a differentially expressed protein. In this manner, antibodies can be used to detect the presence of any peptide which shares one or more antigenic determinants with a differentially expressed protein.

The screening assay can also involve monitoring the expression of a differentially expressed protein. For example, regulators of expression of a differentially expressed protein can be identified in a method in which a cell is contacted with a candidate compound and the expression of a differentially expressed protein or mRNA in the cell is determined. The level of expression of a differentially expressed protein or mRNA the presence of the candidate compound is compared to the level of expression of a differentially expressed protein or mRNA in the absence of the candidate compound. The candidate compound can then be identified as a regulator of expression of a differentially expressed protein based on this comparison. For example, when expression of a differentially expressed protein or mRNA protein is greater (statistically significantly greater) in the presence of the candidate compound than in its absence, the candidate compound is identified as a stimulator of a differentially expressed protein or mRNA expression. Alternatively, when expression of a differentially expressed protein or mRNA is less (statistically significantly less) in the presence of the candidate compound than in its absence, the candidate compound is identified as an inhibitor of a differentially expressed protein or mRNA expression. The level of a differentially expressed protein or mRNA expression in the cells can be determined by methods described below.

Screening for Therapeutic Agents Using Binding Assays

For binding assays, the test compound is preferably a small molecule which binds to and occupies the active site of a differentially expressed protein polypeptide, thereby making the ligand binding site inaccessible to substrate such that normal biological activity is prevented. Examples of such small molecules include, but are not limited to, small peptides or peptide-like molecules. Potential ligands which bind to a polypeptide of the invention include, but are not limited to, the natural ligands of known differentially expressed protein PDEs and analogues or derivatives thereof.

In binding assays, either the test compound or the differentially expressed polypeptide can comprise a detectable label, such as a fluorescent, radioisotopic, chemiluminescent, or enzymatic label, such as horseradish peroxidase, alkaline phosphatase, or luciferase. Detection of a test compound which is bound to differentially expressed polypeptide can then be accomplished, for example, by direct counting of radioemmission, by scintillation counting, or by determining conversion of an appropriate substrate to a detectable product. Alternatively, binding of a test compound to a differentially expressed polypeptide can be determined without labeling either of the interactants. For example, a microphysiometer can be used to detect binding of a test compound with a differentially expressed polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a differentially expressed protein [Haseloff, (1988)].

Determining the ability of a test compound to bind to differentially expressed protein also can be accomplished using a technology such as real-time Bimolecular Interaction Analysis (BIA) [McConnell, (1992); Sjolander, (1991)]. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a differentially expressed protein-like polypeptide can be used as a “bait protein” in a two-hybrid assay or three-hybrid assay [Szabo, (1995); U.S. Pat. No. 5,283,317), to identify other proteins which bind to or interact with a differentially expressed protein and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a differentially expressed protein can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with a differentially expressed protein.

It may be desirable to immobilize either the differentially expressed protein (or polynucleotide) or the test compound to facilitate separation of the bound form from unbound forms of one or both of the interactants, as well as to accommodate automation of the assay. Thus, either the differentially expressed protein-like polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the differentially expressed protein-like polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to the differentially expressed protein (or a polynucleotide encoding for the differentially expressed protein) can be accomplished in any vessel suitable for containing the reactants. Examples of such vessels include microtiter plates, test tubes, and microcentrifuge tubes.

In one embodiment, the differentially expressed protein is a fusion protein comprising a domain that allows binding of the differentially expressed protein to a solid support. For example, glutathione-5-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed differentially expressed protein; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either the differentially expressed protein (or a polynucleotide encoding the differentially expressed protein) or a test com-pound can be immobilized utilizing conjugation of biotin and streptavidin. Biotinylated differentially expressed protein (or a polynucleotide encoding biotinylated differentially expressed protein) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated plates (Pierce Chemical). Alternatively, antibodies which specifically bind to the differentially expressed protein, polynucleotide, or a test compound, but which do not interfere with a desired binding site, such as the active site of the differentially expressed protein, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the differentially expressed protein or test compound, enzyme-linked assays which rely on detecting an activity of the differentially expressed protein, and SDS gel electrophoresis under non-reducing conditions.

Screening for test compounds which bind to the differentially expressed protein or poly-nucleotide also can be carried out in an intact cell. Any cell which comprises the differentially expressed polypeptide or polynucleotide can be used in a cell-based assay system. A differentially expressed protein polynucleotide can be naturally occurring in the cell or can be introduced using techniques such as those described above. Binding of the test compound to the differentially expressed protein or a polynucleotide encoding the differentially expressed protein is determined as described above.

Functional Assays

Test compounds can be tested for the ability to increase or decrease activity of a differentially expressed polypeptide. The differentially expressed protein activity can be measured, for example, using methods described in the specific examples, below. differentially expressed protein activity can be measured after contacting either a purified differentially expressed protein or an intact cell with a test compound. A test compound which decreases the differentially expressed protein activity by at least about 10, preferably about 50, more preferably about 75, 90, or 100% is identified as a potential agent for decreasing the differentially expressed protein activity. A test compound which increases the differentially expressed protein activity by at least about 10, preferably about 50, more preferably about 75, 90, or 100% is identified as a potential agent for increasing the differentially expressed protein activity.

Gene Expression

In another embodiment, test compounds which increase or decrease the differentially expressed protein gene expression are identified (i.e., test compounds which increase or decrease the expression of a differentially expressed polynucleotide sequence of the invention). As used herein, the term “correlates with expression of a poly-nucleotide” indicates that the detection of the presence of nucleic acids, the same or related to a nucleic acid sequence encoding the differentially expressed protein, by northern analysis or realtime PCR is indicative of the presence of nucleic acids encoding the differentially expressed protein in a sample, and thereby correlates with expression of the transcript from the polynucleotide encoding the differentially expressed protein. The term “microarray”, as used herein, refers to an array of distinct polynucleotides or oligonucleotides arrayed on a substrate, such as paper, nylon or any other type of membrane, filter, chip, glass slide, or any other suitable solid support. A differentially expressed protein polynucleotide is contacted with a test compound, and the expression of an RNA or polypeptide product of the differentially expressed protein polynucleotide is determined. The level of expression of appropriate mRNA or polypeptide in the presence of the test compound is compared to the level of expression of mRNA or polypeptide in the absence of the test compound. The test compound can then be identified as a regulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of the differentially expressed protein mRNA or polypeptide expression in the cells can be determined by methods well known in the art for detecting mRNA or polypeptide. Either qualitative or quantitative methods can be used. The presence of polypeptide products of the differentially expressed protein polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into the differentially expressed protein.

Such screening can be carried out either in a cell-free assay system or in an intact cell. Any cell which expresses the differentially expressed protein polynucleotide can be used in a cell-based assay system. The differentially expressed protein polynucleotide can be naturally occurring in the cell or can be introduced using techniques such as those described above. Either a primary culture or an established cell line can be used.

Screening of Therapeutic Agents Against Pain-Specific Array

In one embodiment the present invention provides a method for screening agents for their ability to regulate the expression of genes which are differentially expressed in an animal subjected to pain. In brief, the method comprises administering to an animal subjected to pain, such as an animal pain model, a potentially therapeutic agent, isolating nucleic acid from sensory neurons of the animal, preparing the nucleic acid for hybridization to a microarray as described above, and hybridizing the nucleic acid to a pain-specific microarray. The hybridization level is then compared to the hybridization of a nucleic acid sample contacted with the pain-specific microarray obtained from an animal subjected to pain, but not administered the potentially therapeutic agent. In one embodiment, the potentially therapeutic agent is deemed to be therapeutic if the expression level of the nucleic acid sequence obtained from the animal subjected to pain and treated with the agent is no longer differentially expressed by at least 1.4 fold, and wherein the expression of the nucleic acid sequence obtained from the animal subjected to pain but not treated with the agent remains differentially regulated. The nucleic acid sequences analyzed to determine therapeutic efficacy can include any of the sequences previously identified (see above) as being differentially expressed in an animal subjected to pain.

Animals may be administered any potentially therapeutic agent known in the art, including antisense molecules, ribozymes, and supplemental nucleic acid sequences as described above. Additional therapeutic agents include any agent known in the art which is routinely administered for the amelioration of pain including, but not limited to aspirin, ibuprofen, narcotics, steroidial and non-steroidial anti-inflammatories, and the like. These agents are administered according to dosing protocols well known in the art.

Screening of Therapeutic Agents Against Individual Genes that are Differentially Expressed in Pain

Candidate therapeutic agents of the invention are screened for their ability to regulate the expression of one or more isolated polynucleotide sequences which have been identified herein as differentially regulated in an animal which has been subjected to pain relative to an animal that is not subjected to pain. In one embodiment, the screen consists of administering a candidate therapeutic agent, as defined herein, or a placebo, to an animal that is subjected to pain and hybridizing a nucleic acid sample, corresponding to RNA obtained from such a treated or non treated animal, to a probe specific for a polynucleotide sequence selected from the group of isolated polynucleotide sequences of Tables 1, 2, 3, 4, or 5. In another embodiment, the screen consists of administering a candidate therapeutic agent, as defined herein, or a placebo, to an in vitro cell culture of primary cells for example, primary neurons, that naturally express polynucleotide sequences selected from the group of isolated polynucleotide sequences of Tables 1, 2, 3, 4, or 5. In a further embodiment, the screen consists of administering a candidate therapeutic agent, as defined herein, or a placebo, to cell lines that have been transfected with vectors that direct the expression of polynucleotide sequences selected from the group of isolated polynucleotide sequences of Tables 1, 2, 3, 4, or 5. In a further embodiment, the screen consists of administering a candidate therapeutic agent, as defined herein, or a placebo, to a transgenic animal in which a neural specific promoter drives the expression of a polynucleotide sequence selected from the group of isolated polynucleotide sequences of Tables 1, 2, 3, 4, or 5. In all instances, a 10% increase or decrease in the differential expression of a gene in response to a therapeutic compound is indicative of a therapeutic agent that can modulate the differential expression of a gene that is differentially regulated in an animal which has been subjected to pain relative to an animal that is not subjected to pain. In a preferred embodiment, nucleic acid samples obtained from treated and non-treated animals or in vitro cell cultures are hybridized to 1 or more, 2 or more, 5 or more, 50 or more, 100 or more, 500 or more, 1000 or more probes, each probe being specific to a polynucleotide sequence selected from the group of differentially expressed polynucleotide sequences of Tables 1, 2, 3, 4, or 5.

Methods for measuring the differential expression of one or more of the polynucleotides sequences of Tables 1, 2, 3, 4, or 5 in nucleic acid samples from treated animals relative to non-treated animals, are well known in the art and include, but are not limited to, reverse transcription PCR (RT-PCR; described in U.S. Pat. No. 5,4078,00), Taqman (as disclosed in U.S. Pat. Nos. 5,210,015 and 5,487,972), Molecular Beacon assays (as disclosed in WO 95/13399), Northern blot hybridization, S1 nuclease mapping, RNAse protection assays which are described in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995). References to patents and literature are by incorporated in their entirety.

Compounds identified as positives based on this screen can be further tested for activity in the in vitro cell culture assay, in vivo protein activity assay or analgesic assays, described herein, to determine if these compounds are effective at modulating differential gene expression in response to pain and ultimately attenuating pain itself.

Polypeptide Activity

In one embodiment, the present invention provides a method for screening potentially therapeutic agents which modulate the activity of one or more polypeptides encoded by one or more of the polynucleotide sequences in Tables 1, 2, 3, 4, or 5, such that if the activity of the polypeptide is increased in an animal subjected to pain, the therapeutic substance will decrease the activity of the polypeptide relative to the activity of the same polypeptide in an animal subjected to pain, but not treated with the therapeutic agent. Likewise, if the activity of the polypeptide is decreased in an animal subjected to pain, the therapeutic substance will increase the activity of the polypeptide relative to the activity of the same polypeptide in an animal subjected to the same pain, but not treated with the therapeutic agent.

The activity of the polypeptide molecules encoded by the polynucleotides indicated in Tables 1, 2, 3, 4, or 5 may be measured by any means known to those of skill in the art, and which are particular for the type of activity performed by the particular polypeptide. Examples of specific assays which may be used to measure the activity of particular polynucleotide products are shown below.

(a) G-protein Coupled Receptors

In one embodiment, the one or more of the differentially regulated polynucleotides of Tables 1, 2, 3, 4, or 5 may encode a G-protein coupled receptor. In one embodiment, the present invention provides a method of screening potential agonists and antagonists of the family of G-protein coupled receptors, including Gₛ, Gᵢ, and G_{q}, encoded by the differentially expressed polynucleotides of the present invention by measuring changes in the activity of these receptors in the presence of a candidate agonist or antagonist.

1. Gᵢ-Coupled Receptor Screening

Cells (such as CHO cells, or primary cells) are stably transfected with the relevant receptor and with an inducible CRE-luciferase construct. Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 2000 cells/well in 35 μl cell culture medium) in DMEM/FI2 with FBS, and are grown for 48 hours (range: ˜24-60 hours, depending on cell line). Growth medium is then exchanged against serum free medium (SFM; e.g. Ultra-CHO), containing 0,1% BSA. Test compounds dissolved in DMSO are diluted in SFM and transferred to the test cultures (maximal final concentration 10 μmolar), followed by addition of forskolin (˜1 molar, final conc.) in SFM+0,1% BSA 10 minutes later. In case of antagonist screening both, an appropriate concentration of agonist, and forskolin are added. The plates are incubated at 37° C. in 10% CO₂ for 3 hours. Then the supernatant is removed, cells are lysed with lysis reagent (25 mmolar phosphate-buffer, pH 7,8, containing 2 mmolar DDT, 10% glycerol and 3% Triton X100). The luciferase reaction is started by addition of substrate-buffer (e.g. luciferase assay reagent, Promega) and luminescence is immediately determined (e.g. Berthold luminometer or Hamamatzu camera system).

2. Gₛ-Coupled Receptor Screening

Cells (such as CHO, or primary cells) are stably transfected with the relevant receptor and with an inducible CRE-luciferase construct. Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 1000 or 2000 cells/well in 35 μl cell culture medium) in DMEM/F12 with FBS, and are grown for 48 hours (range: ˜24-60 hours, depending on cell line). The assay is started by addition of test-compounds in serum free medium (SFM; e.g. Ultra-CHO) containing 0,1% BSA: Test compounds are dissolved in DMSO, diluted in SFM and transferred to the test cultures (maximal final concentration 10 μmolar, DMSO conc. <0,6%). In case of antagonist screening an appropriate concentration of agonist is added 5-10 minutes later. The plates are incubated at 37° C. in 10% CO₂ for 3 hours. Then the cells are lysed with 10 μl lysis reagent per well (25 mmolar phosphate-buffer, pH 7,8, containing 2 mmolar DDT, 10% glycerol and 3% Triton X100) and the luciferase reaction is started by addition of 20 μl substrate-buffer per well (e.g. luciferase assay reagent, Promega). Measurement of luminescence is started immediately (e.g. Berthold luminometer or Hamamatzu camera system).

3. G_{q}-Coupled Receptor Screening

Cells (such as CHO, or primary cells) are stably transfected with the relevant receptor. Cells expressing functional receptor protein are grown in 50% Dulbecco's modified Eagle medium/50% F12 (DMEM/F12) supplemented with 10% FBS, at 37° C. in a humidified atmosphere with 5% CO₂ and are routinely split at a cell line dependent ratio every 3 or 4 days. Test cultures are seeded into 384-well plates at an appropriate density (e.g. 2000 cells/well in 35 μl cell culture medium) in DMEM/F12 with FBS, and are grown for 48 hours (range: ˜24-60 hours, depending on cell line). Growth medium is then exchanged against physiological salt solution (e.g. Tyrode solution). Test compounds dissolved in DMSO are diluted in Tyrode solution containing 0.1% BSA and transferred to the test cultures (maximal final concentration 10 μmolar). After addition of the receptor specific agonist the resulting Gq-mediated intracellular calcium increase is measured using appropriate read-out systems (e.g. calcium-sensitive dyes).

(b) Ion Channels

Ion channels are integral membrane proteins involved in electrical signaling, transmembrane signal transduction, and electrolyte and solute transport. By forming macromolecular pores through the membrane lipid bilayer, ion channels account for the flow of specific ion species driven by the electrochemical potential gradient for the permeating ion. At the single molecule level, individual channels undergo conformational transitions (“gating”) between the ‘open’ (ion conducting) and ‘closed’ (non conducting) state. Typical single channel openings last for a few milliseconds and result in elementary transmembrane currents in the range of 10-9-10-12 Ampere. Channel gating is controlled by various chemical and/or biophysical parameters, such as neurotransmitters and intracellular second messengers (‘ligand-gated’ channels) or membrane potential (‘voltage-gated’ channels). Ion channels are functionally characterized by their ion selectivity, gating properties, and regulation by hormones and pharmacological agents. Because of their central role in signaling and transport processes, ion channels present ideal targets for pharmacological therapeutics in various pathophysiological settings.

In one embodiment, the one or more of the differentially regulated polynucleotides of Tables 1, 2, 3, 4, or 5 may encode an ion channel. In one embodiment, the present invention provides a method of screening potential activators or inhibitors of channel activity encoded by the differentially expressed polynucleotides of the present invention. Screening for compounds interacting with ion channels to either inhibit or promote their activity can be based on (1.) binding and (2.) functional assays in living cells (see for example, Hille, 1992, Ion Channels of Excitable Membranes Sunderland, Mass., Sinauer Associates, Inc.; incorporated herein by reference in its entirety).

1. For ligand-gated channels, e.g. ionotropic neurotransmitter/hormone receptors, assays can be designed detecting binding to the target by competition between the compound and a labeled ligand.

2. Ion channel function can be tested functionally in living cells. Target proteins are either expressed endogenously in appropriate reporter cells or are introduced recombinantly. Channel activity can be monitored by (2.1) concentration changes of the permeating ion (most prominently Ca2+ ions), (2.2) by changes in the transmembrane electrical potential gradient, and (2.3) by measuring a cellular response (e.g. expression of a reporter gene, secretion of a neurotransmitter) triggered or modulated by the target activity.
2.1. Channel activity results in transmembrane ion fluxes. Thus activation of ionic channels can be monitored by the resulting changes in intracellular ion concentrations using luminescent or fluorescent indicators. Because of its wide dynamic range and availability of suitable indicators this applies particularly to changes in intracellular Ca2+ ion concentration ([Ca2+]i). [Ca2+]i can be measured, for example, by aequorin luminescence or fluorescence dye technology (e.g. using Fluo-3, Indo-1, Fura-2). Cellular assays can be designed where either the Ca2+ flux through the target channel itself is measured directly or where modulation of the target channel affects membrane potential and thereby the activity of co-expressed voltage-gated Ca2+ channels.
2.2. Ion channel currents result in changes of electrical membrane potential (Vm) which can be monitored directly using potentiometric fluorescent probes. These electrically charged indicators (e.g. the anionic oxonol dye DiBAC4(3)) redistribute between extra- and intracellular compartment in response to voltage changes. The equilibrium distribution is governed by the Nernst-equation. Thus changes in membrane potential results in concomitant changes in cellular fluorescence. Again, changes in Vm might be caused directly by the activity of the target ion channel or through amplification and/or prolongation of the signal by channels co-expressed in the same cell.
2.3. Target channel activity can cause cellular Ca2+ entry either directly or through activation of additional Ca2+ channel (see 2.1). The resulting intracellular Ca2+ signals regulate a variety of cellular responses, e.g. secretion or gene transcription. Therefore modulation of the target channel can be detected by monitoring secretion of a known hormone/transmitter from the target-expressing cell or through expression of a reporter gene (e.g. luciferase) controlled by an Ca2+-responsive promoter element (e.g. cyclic AMP/Ca2+-responsive elements; CRE).

(c) Transcription Factors

In one embodiment, one or more of the differentially expressed polynucleotide sequences of Tables 1, 2, 3, 4, or 5 may encode a transcription factor. The activity of such a transcription factor may be measured, for example, by a promotor assay which measures the ability of the transcription factor to initiate transcription of a test sequence linked to a particular promotor. In one embodiment, the present invention provides a method for screening a test compound for its ability to modulate the activity of such a transcription factor by measuring the changes in the expression of a test gene which is regulated by a promoter which is responsive to the transcription factor.

A promoter assay can be set up with a human hepatocellular carcinoma cell HepG2 that is stably transfected with a luciferase gene under the control of a X (e.g. thyroid hormone) regulated promoter. The vector 2xIROluc, which can be used for transfection, carries a thyroid hormone responsive element (TRE) of two 12 bp inverted palindromes separated by an 8 bp spacer in front of a tk minimal promoter and the luciferase gene.

Test cultures are seeded in 96 well plates in serum—free Eagle's Minimal Essential Medium supplemented with glutamine, tricine, sodium pyruvate, non-essential amino acids, insulin, selen, transferrin, and are cultivated in a humidified atmosphere at 10% CO₂ at 37° C. After 48 hours of incubation serial dilutions of test compounds or reference compounds (L-T3, L-T4 e.g.) and costimulator if appropriate (final concentration 1 nM) are added to the cell cultures and incubation is continued for the optimal time (e.g. another 4-72 hours). The cells are then lysed by addition of buffer containing Triton X100 and luciferin and the luminescence of luciferase induced by T3 or other compounds is measured in a luminometer. For each concentration of a test compound replicates of 4 can be tested. EC50—values for each test compound can be calculated by use of, for example, the Graph Pad Prism Scientific software.

Screening of Therapeutic Agents that Modulate the In Vivo Activity of Proteins Encoded by Genes That are Differentially Expressed in Pain

The invention further provides for a screen of therapeutic compounds that modulate the in vivo activity of proteins encoded by genes that are differentially expressed in an animal subjected to pain (see Tables 1, 2, 3, 4, or 5). Methods for measuring changes in the in vivo activity of the proteins of the invention are well known in the art and include, but are not limited to, testing for changes in enzymatic activity, G coupled receptor activity or ion channel activity (as described herein under Polypeptide Activity); transcription factor function or the activity of signal tranduction pathway intermediates. Generally, these methods involve administering a candidate compound, as defined herein, or a placebo, to an animal that has been subjected to pain, preparing protein extracts from neural tissues and testing for a modulation in the protein activity in the extract in response to the candidate compound. In one embodiment, “protein activity” refers to the activity of a protein that is encoded by a gene that has been identified as a gene that is differentially expressed in an animal subjected to pain. In another embodiment, “protein activity” refers to the activity of one or more proteins whose activity is modulated by a protein that is encoded by a gene that has been identified as a gene that is differentially expressed in an animal subjected to pain.

In one embodiment, the “protein activity”, according to the invention, refers to the ability of one or more ligands to bind to cell surface receptors that are differentially expressed in animals subjected to pain. For example, WO0102566A1 describes a screen for compounds that modulate the binding of glutamate to glutamate binding receptors.

In another embodiment, the “protein activity”, according to the invention, is controlled by post-translational protein modification, e.g. phosphorylation or dephosphorylation. For example the protein, identified as being encoded by a gene that is differentially expressed in animals subjected to pain, may be a kinase, whose activity is modulated in response to a candidate compound either by direct phosphorylation or dephosphorylation. Alternatively, the activity of the kinase can be determined by assaying the phosphorylation of one or more substrates of the kinase. Methods for measuring the phosphorylation state of a protein are well known to a person skilled in the art. Typically radioactive phosphate is administered to a test animal that is then subjected to pain in the presence or absence of a therapeutic compound. Protein extracts are then prepared from neurological tissues and the protein of interest is isolated by immunoprecipitation and analyzed by SDS polyacrylamide electrophoresis. A 10% or more increase or decrease in the level of phosphorylation of the protein of interest in the presence of a compound relative to the level of phosphorylation in the absence of the compound is indicative of a compound that modulates the “protein activity”.

More generally, a gene, that is differentially expressed in animals subjected to pain, may encode a kinase or phosphatase that is part of a signal transduction pathway known in the art. If so, modulation of the activity of the kinase or phosphatase in response to a candidate compound can be determined by assaying the activity of pathway intermediates that are found downstream of the kinase or phosphatase in the pathway. For example, the activity of a kinase or phosphatase can be determined by measuring effects on gene expression or transcription factor activity. Methods for measuring differential gene expression or transcription factor function are well known in the art and are described supra. For example, the binding activity of a transcription factor to its cognate DNA binding site can be tested in protein extracts derived from treated animals using a mobility shift type analysis (see, e.g., Sambrook, Fritsch & Maniatis, 1989, Molecular Cloning: A Laboratory Manual, Second Edition; Short Protocols In Molecular Biology, (Ausubel et al., ed., 1995)). In addition, the ability of a transcription factor to activate transcription from a promoter containing one or more cognate DNA binding sites can also be tested using standard reporter type assays (GFP, CAT, lacZ) that are also well known in the art (See Ausubel et al; supra).

Modeling of Regulators

Computer modeling and searching technologies permit identification of compounds, or the improvement of already identified compounds, that can modulate the differentially expressed protein expression or activity. Having identified such a compound or composition, the active sites or regions are identified. Such sites might typically be the enzymatic active site, regulator binding sites, or ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential the differentially expressed protein modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Diagnostic Assays

The present invention provides a method for detecting pain in an animal suspected of having pain, wherein the amount of one or more polynucleotide and/or polypeptide sequences described herein, particularly in Table 11, is measured in the animal and compared with the amount measured in an animal which is not suspected of having pain. If an increase or decrease in the amount of the polynucleotide and/or polypeptide sequence of at least 1.2 fold and statistically significant at P<0.05 is measured, then the animal suspected of having pain is identified as having pain. Preferably the amount of a polynucleotide and/or polypeptide sequence measured in an animal suspected of having pain will be increased or decreased by 1.4 fold relative to the amount measured in an animal not suspected of having pain.

The amount of a polynucleotide and/or polypeptide sequence described herein may be measured from any tissue/fluid in an animal, but is preferably measured in a sensory neuron such as a DH neuron or DRG neuron. In one embodiment, a polynucleotide/polypeptide sequence of the invention is expressed in a sensory neuron and then secreted into the blood, lymph, or CSF, wherein it is subsequently detected. Alternatively, or in addition, a polypeptide and/or polynucleotide sequence can be measured in a “patient sample” which includes, but is not limited to blood, serum, plasma, and cerebrospinal fluid.

Sensory neuron and/or patient samples may be obtained and utilized according to methods which are well known in the art, and screened, according to the invention, to detect the presence of one or more of the sequences of Tables 1-11 by methods which are well known in the art. “Detecting” as used herein refers to the identification of the presence or absence of a molecule in a sample. Where the molecule to be detected is a polypeptide, the step of detecting can be performed by binding the polypeptide with an antibody that is detectably labeled. A detectable label is a molecule which is capable of generating, either independently, or in response to a stimulus, an observable signal. A detectable label can be, but is not limited to a fluorescent label, a chromogenic label, a luminescent label, or a radioactive label. Methods for “detecting” a label include quantitative and qualitative methods adapted for standard or confocal microscopy, FACS analysis, and those adapted for high throughput methods involving multiwell plates, arrays or microarrays. One of skill in the art can select appropriate filter sets and excitation energy sources for the detection of fluorescent emission from a given fluorescent polypeptide or dye. “Detecting” as used herein can also include the use of multiple antibodies to a polypeptide to be detected, wherein the multiple antibodies bind to different epitopes on the polypeptide to be detected. Antibodies used in this manner can employ two or more detectable labels, and can include, for example a FRET pair. A polypeptide molecule, such as the a polypeptide of Table 11, is “detected” according to the present invention when the level of detectable signal is at all greater than the background level of the detectable label, or where the level of measured nucleic acid is at all greater than the level measured in a control sample.

As used herein, “detecting” as it refers to detecting the presence of a target nucleic acid molecule (e.g., a nucleic acid molecule shown in Table 11) refers to a process wherein the signal generated by a directly or indirectly labeled probe nucleic acid molecule (capable of hybridizing to a target, e.g., a sequence of Table 11, in a serum sample) is measured or observed. Thus, detection of the probe nucleic acid is directly indicative of the presence, and thus the detection, of a target nucleic acid. For example, if the detectable label is a fluorescent label, the target nucleic acid is detected by observing or measuring the light emitted by the fluorescent label on the probe nucleic acid when it is excited by the appropriate wavelength, or if the detectable label is a fluorescence/quencher pair, the target nucleic acid is detected by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is detected by, for example, autoradiography. Methods and techniques for detecting fluorescent, radioactive, and other chemical labels may be found in Ausubel et al. (1995, Short Protocols in Molecular Biology, 3^{rd} Ed. John Wiley and Sons, Inc.). Alternatively, a nucleic acid may be indirectly detected wherein a moiety is attached to a probe nucleic acid which will hybridize with the target, such as an enzyme activity, allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers which are specifically designed to hybridize with a portion of the target nucleic acid sequence. Quantative amplification methods, such as, but not limited to TaqMan, may also be used to detect a target nucleic acid according to the invention. A nucleic acid molecule is detected as used herein where the level of nucleic acid measured (such as by quantitative PCR), or the level of detectable signal provided by the detectable label is at all above the background level.

In clinical applications, animal tissue samples, preferably serum, can be screened for the presence and/or absence of one or more of the sequences of the invention, and in particular, of Table 11. Such samples may comprise tissue samples, whole cells, cell lysates, or isolated nucleic acids, including, for example, needle biopsy cores, surgical resection samples, lymph node tissue, cerebrospinal fluid, or serum. A sample for analysis as described herein is preferably a serum sample. A serum sample may be obtained from an animal using methods which are well known to those of skill in the art. Briefly, a whole venous or arterial blood sample from an animal is collected into a test tube. The whole blood sample is permitted to incubate at room temperature for approximately 15-30 to allow the blood to clot. Once clotted, the sample is centrifuged at approximately 1500 to 3000 rpm for 5-30 minutes to completely separate the serum from the cellular components. This centrifugation may be repeated if necessary to achieve complete separation. The resulting serum sample may be subsequently screened for the presence of one or more of the nucleic acid or amino acid sequences described herein.

Analgesia Assays: In Vivo Testing of Compounds/Target Validation for Pain Treatment

Acute Pain

Acute pain is measured on a hot plate mainly in rats. Two variants of hot plate testing are used: In the classical variant animals are put on a hot surface (52 to 56° C.) and the latency time is measured until the animals show nocifensive behavior, such as stepping or foot licking. The other variant is an increasing temperature hot plate where the experimental animals are put on a surface of neutral temperature. Subsequently this surface is slowly but constantly heated until the animals begin to lick a hind paw. The temperature which is reached when hind paw licking begins is a measure for pain threshold.

Compounds are tested against a vehicle treated control group. Substance application is performed at different time points via different application routes (intravenous (i.v.), intra-peritoneal (i.p.), by mouth (p.o.), by inhalation (i.t.), Intracerebroventricular (i.c.v.), subcutaneous (s.c.), intradermal, or transdermal) prior to pain testing.

According to the invention, a candidate compound, may be administered to an animal which is subjected to an acute pain assay. Acute pain, measured according to the above assay, decreased by at least 10%, and preferably 20%, 40%, 60%, and up to 100% is then indicative of a candidate compound that decreases pain.

Persistent Pain

Persistent pain is measured with the formalin or capsaicin test, mainly in rats. A solution of 1 to 5% formalin or 10 to 100 μg capsaicin is injected into one hind paw of the experimental animal. After formalin or capsaicin application the animals show nocifensive reactions like flinching, licking and biting of the affected paw. The number of nocifensive reactions within a time frame of up to 90 minutes is a measure for intensity of pain.

Compounds are tested against a vehicle treated control group. Substance application is performed at different time points via different application routes (i.v., i.p., p.o., i.t., i.c.v., s.c., intradermal, transdermal) prior to formalin or capsaicin administration.

According to the invention, a candidate compound, may be administered to an animal which is subjected to an persistent pain assay. Persistent pain, measured according to the above assay, decreased by at least 10% and preferably 20%, 40%, 60%, and up to 100% is then indicative of a candidate compound that decreases pain.

Neuropathic Pain

Neuropathic pain is induced by different variants of unilateral sciatic nerve injury mainly in rats. The operation is performed under anesthesia. The first variant of sciatic nerve injury is produced by placing loosely constrictive ligatures around the common sciatic nerve (Bennett and Xie, Pain 33 (1988): 87-107). The second variant is the tight ligation of about the half of the diameter of the common sciatic nerve (Seltzer et al., Pain 43 (1990): 205-218). In the next variant, a group of models is used in which tight ligations or transections are made of either the L5 and L6 spinal nerves, or the L5 spinal nerve only (Kim S H; Chung Jm, An experimental-model for peripheral neuropathy produced by segmental spinal nerve ligation in the rat, Pain 50 (3) (1992): 355-363). The fourth variant involves an axotomy of two of the three terminal branches of the sciatic nerve (tibial and common peroneal nerves) leaving the remaining sural nerve intact whereas the last variant comprises the axotomy of only the tibial branch leaving the sural and common nerves uninjured. Control animals are treated with a sham operation.

Postoperatively, the nerve injured animals develop a chronic mechanical allodynia, cold allodynioa, as well as a thermal hyperalgesia. Mechanical allodynia is measured by means of a pressure transducer (electronic von Frey Anesthesiometer, IITC Inc.-Life Science Instruments, Woodland Hills, SA, USA; Electronic von Frey System, Somedic Sales AB, Horby, Sweden). Thermal hyperalgesia is measured by means of a radiant heat source (Plantar Test, Ugo Basile, Comerio, Italy), or by means of a cold plate of 5 to 10° C. where the nocifensive reactions of the affected hind paw are counted as a measure of pain intensity. A further test for cold induced pain is the counting of nocifensive reactions, or duration of nocifensive responses after plantar administration of acetone to the affected hind limb. Chronic pain in general is assessed by registering the circadanian rhytms in activity (Surjo and Arndt, Universitat zu Koln, Cologne, Germany), and by scoring differences in gait (foot print patterns; FOOTPRINTS program, Klapdor et al., 1997. A low cost method to analyze footprint patterns. J. Neurosci. Methods 75, 49-54).

Compounds are tested against sham operated and vehicle treated control groups. Substance application is performed at different time points via different application routes (i.v.,

i.p., p.o., i.t., i.c.v., s.c., intradermal, transdermal) prior to pain testing.

According to the invention, a candidate compound, may be administered to an animal, which is subjected to an neuropathic pain assay. Neuropathic pain, measured according to the above assay, decreased by at least 10% and preferably 20%, 40%, 60%, and up to 100% is then indicative of a candidate compound that decreases pain.

Inflammatory Pain

Inflammatory pain is induced mainly in rats by injection of 0.75 mg carrageenan or complete Freund's adjuvant into one hind paw. The animals develop an edema with mechanical allodynia as well as thermal hyperalgesia. Mechanical allodynia is measured by means of a pressure transducer (electronic von Frey Anesthesiometer, IITC Inc.-Life Science Instruments, Woodland Hills, SA, USA). Thermal hyperalgesia is measured by means of a radiant heat source (Plantar Test, Ugo Basile, Comerio, Italy, Paw thermal stimulator, G. Ozaki, University of California, USA). For edema measurement two methods are being used. In the first method, the animals are sacrificed and the affected hindpaws sectioned and weighed. The second method comprises differences in paw volume by measuring water displacement in a plethysmometer (Ugo Basile, Comerio, Italy).

Compounds are tested against uninflamed as well as vehicle treated control groups. Substance application is performed at different time points via different application routes (i.v., i.p., p.o., i.t., i.c.v., s.c., intradermal, transdermal) prior to pain testing.

According to the invention, a candidate compound, may be administered to an animal which is subjected to an inflammatory pain assay. Inflammatory pain, measured according to the above assay, decreased by at least 10% and preferably 20%, 40%, 60%, and up to 100% is then indicative of a candidate compound that decreases pain.

Diabetic Neuropathic Pain

Rats treated with a single intraperitoneal injection of 50 to 80 mg/kg streptozotocin develop a profound hyperglycemia and mechanical allodynia within 1 to 3 weeks. Mechanical allodynia is measured by means of a pressure transducer (electronic von Frey Anesthesiometer, IITC Inc.-Life Science Instruments, Woodland Hills, SA, USA).

Compounds are tested against diabetic and non-diabetic vehicle treated control groups. Substance application is performed at different time points via different application routes (i.v.,

i.p., p.o., i.t., i.c.v., s.c., intradermal, transdermal) prior to pain testing.

According to the invention, a candidate compound, may be administered to an animal which is subjected to an Diabetic Neuropathic pain assay. Diabetic Neuropathic pain, measured according to the above assay, decreased by at least 10% and preferably 20%, 40%, 60%, and up to 100% is then indicative of a candidate compound that decreases pain.

In one embodiment, the candidate compounds which are administered to an animal subjected to one or more of the above pain stimuli, can be a candidate compound which had been previously determined to regulate the expression of one or more of the differentially expressed polynucleotide sequences indicated in Tables 1, 2, 3, 4, or 5, and/or previously determined to regulate the activity of a protein encoded by one or more of the differentially expressed polynucleotides indicated in Table 1, 2, 3, 4, or 5.

Dosage and Administration

Therapeutic agents of the invention are administered to an animal, preferably in a biologically compatible solution or a pharmaceutically acceptable delivery vehicle, by ingestion, injection, inhalation or any number of other methods. For embodiments where the therapeutic agent is a vector comprising an antisense sequence, a sequence encoding a ribozyme, or a sequence designed to supplement a down regulated sequence in an animal subjected to pain, the vectors may be administered as a pharmaceutical formulation, or may be administered using any method known in the art including microinjection, transfection, transduction, and ex vivo delivery. The dosages administered will vary from patient to patient; a “therapeutically effective dose” is determined, for example but not limited to, by the level of enhancement of function (e.g., for a nucleic acid sequence which is overexpressed by at least 1.4 fold in an animal subjected to pain relative to a naïve animal, a therapeutically effective dose is one which reduces the level of overexpression of the sequence to less than 1.4 fold. The converse would define a therapeutically effective dose for increasing the expression of an under-expressed sequence).

A therapeutic agent according to the invention is preferably administered in a single dose. This dosage may be repeated daily, weekly, monthly, yearly, or until the nucleic acid sequence is no longer differentially expressed.

Pharmaceutical Compositions

The invention provides for compositions comprising a therapeutic agent according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which is used pharmaceutically.

Pharmaceutical compositions for oral administration are formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use are obtained through a combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which are used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and are formed with many acids, including but not limited to hydrochloric, sulfuric, acetic, lactic, tartaric, malic, succinic, etc. . . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a therapeutic agent of the invention formulated in a acceptable carrier have been prepared, they are placed in an appropriate container and labeled for treatment of an indicated condition with information including amount, frequency and method of administration.

### EXAMPLES

The examples below are non-limiting and are merely representative of various aspects and features of the present invention.

### Example 1

### Identification of Differentially Expressed Nucleic Acid Sequences

The present invention relates to a method for the identification of nucleic acid sequences and/or genes which are differentially expressed in an animal which has been subjected to pain. In one embodiment, the animal is a pain model, that is, the animal has been artificially manipulated such that it meets the criteria for a state of pain as described above. In one embodiment the animal pain model is produced by transection of the sciatic nerve (axotomy). In an alternate embodiment, the animal pain model is the spared nerve injury model (SNI; Decosterd and Woolf, 2000 Pain 87: 149) in which one of the terminal branches of the sciatic nerve is spared from axotomy. In a further alternate embodiment, the animal pain model is an inflammation model (Stein et al., (1988) Pharmacol Biochem Behav 31: 445-451; Woolf et al., (1994) Neurosci. 62, 327-331) in which an irritant such as CFA is injected into an animal to induce inflammation.

Animal Pain Models

Axotomy of the sciatic nerve was performed on adult (200-250 g) male Sprague-Dawley rats. Under halothane (2%) anesthesia, the skin on the lateral surface of the thigh was incised and an incision made directly through the biceps femoris muscle exposing the sciatic nerve. The axotomy procedure involves transecting the sciatic nerve following ligation. The sciatic nerve was tight-ligated with 5.0 silk and sectioned distal to the ligation, removing 2-4 mm of the distal nerve stump. Great care was taken to avoid any contact with or transection of any collateral branches of the sciatic nerve proximal to the transection site, or any cutaneous nerve branches. Muscle and skin were closed in two layers, and animals were allowed to recover for 3-5 days prior to testing for signs of pain including mechanical allodynia, mechanical hyperalgesia, cold allodynia, and heat hyperalgesia using the criteria described above. Sham control animals (naïve) involved exposure of the sciatic nerve and its branched without any lesion.

The SNI nerve injury model was performed on adult (200-250 g) male Sprague-Dawley rats. Under halothane (2%) anesthesia, the skin on the lateral surface of the thigh was incised and a section made directly through the biceps femoris muscle exposing the sciatic nerve and its three terminal branches: the sural, common peroneal and tibial nerves.

The SNI procedure comprises an axotomy and ligation of the tibial and common peroneal nerves leaving the sural nerve intact. The common peroneal and the tibial nerves were tight-ligated with 5.0 silk and sectioned distal to the ligation, removing 2-4 mm of the distal nerve stump. Great care was taken to avoid any contact with or stretching of the intact sural nerve. Muscle and skin were closed in two layers and animals were allowed to recover for at least one week prior to testing for signs of pain including mechanical allodynia, mechanical hyperalgesia, cold allodynia, and heat hyperalgesia using the criteria described above. Sham control animals (naïve) involved exposure of the sciatic nerve and its branched without any lesion.

The inflammation animal pain model was performed on adult male Sprague-Dawley rats (10-11 weeks old, 300-350 g). Inflammation was induced by an intra-plantar injection of complete Freund's adjuvant (CFA, Sigma, 1 μ-1 ml) into the left hind paw of rats under halothane (2.5%) anesthesia, producing an area of erythema, edema and tenderness restricted to the hindpaw (Stein et al., (1988) Pharmacol Biochem Behav 31: 445-451; Woolf et al., (1994) Neurosci. 62, 327-331). Animals were subsequently tested for signs of pain including mechanical allodynia, mechanical hyperalgesia, cold allodynia, and heat hyperalgesia using the criteria described above.

Total RNA Isolation

Following the surgical procedures described above and testing to insure that the axotomy and SNI model animals met the pain criteria described, control and pain model animals were rapidly killed by decapitation. Axotomy model animals were killed 3 days following axotomy, and SNI model animals were killed 10-15 days following surgery.

The dorsal root ganglia (DRG) from spinal levels L4-L5 were removed from the SNI, axotomy, and control animals and snap-frozen in a dry ice/ethanol slurry. DRGs from the two spinal levels were pooled for each animal and total RNA was extracted using Trizol (Invitrogen) according to the manufacturers instructions. Briefly, tissue samples were homogenized in a ground glass homogenizer in 1 ml of Trizol reagent per 50-100 mg of tissue. The samples were incubated for 5 min. at 15-30° C. to permit the complete dissociation of nucleoprotein complexes. Subsequently, 0.2 ml of chloroform was added per 1 ml of Trizol reagent. Samples were agitated and incubated at 15-30° C. for 2 to 3 minutes. Samples were then centrifuged at no more than 12,000×g for 15 minutes at 2-8° C. The aqueous phase was then transferred to a fresh tube and the RNA was precipitated by mixing with 0.5 ml of isopropyl alcohol per 1 ml Trizol reagent used for the initial homogenization. Samples were incubated at 15-30° C. for 10 minutes and centrifuged at 12,000×g for 10 minutes. The supernatant is then removed, and the RNA pellet was washed with 75% ethanol. The RNA pellet is then air dries and resuspended in either RNase-free water or 0.5% SDS solution. The integrity of the RNA samples was verified on a 1% agarose gel, and the RNA was quantified by measuring absorbance at 260/280 mm. cRNA was then prepared from 10 μg of total RNA using techniques that are well known in the art. Briefly, total RNA (7 to 10 μg) was isolated and reverse transcribed using a primer consisting of oligo-dT coupled to a T7 RNA polymerase binding site. The cDNA was made double stranded and biotinylated cRNA was synthesized using T7 polymerase. Unincorporated nucleotides were removed, and the cRNA was quantitated using methods known to those of skill in the art; a yield of cRNA between 25 and 80 μg was typical.

Array Hybridization

The cRNA samples from axotomy, SNI and naïve animals were randomly sheared to an approximate length of 50 nucleotides and subsequently hybridized to an Affymetrix rat genome U34 gene chip set. Briefly, labeled nucleic acid is denatured by heating for 2 minutes at 100° C., and incubated at 37° C. of 20-30 minutes before being placed on a nucleic acid array under a 22 mm×22 mm glass cover slip. Hybridization is carried out at 65° C. for 14 to 18 hours in a custom slide chamber with humidity maintained by a small reservoir of 3×SSC. The array is washed by submersion and agitation for 2-5 min in 2×SSC with 0.1% SDS, followed by 1×SSC, and 0.1×SSC. Finally, the array is dried by centrifugation for 2 minutes in a slide rack in a Beckman GS-6 tabletop centrifuge in Microplus carriers at 650 RPM for 2 min.

External standards were included in each hybridization to control for hybridization efficiency, to test for sensitivity and assist in the comparisons between data sets from different experiments. These external standards are cRNA transcribed from the bacterial genes bio b, bio c, bio d, cre, thr, and phe. The first hybridization was against a Test Chip, which contains probes against human, mouse and yeast mRNAs as well as probes against the exogenously added control RNA. The Test Chips are designed to determine the quality of the cRNA mixture. Stringent washing in the fluidics station reduces non-specific hybridization and the hybridized biotinylated cRNA was detected by incubation with phycoerythrin-streptavidin and was quantitated by scanning using the Hewlett-Packard GeneArray laser scanner. Following positive analysis of the Test Chip, the same hybridization mixture was then added to the Rat Genome U34 gene chip set which monitors the expression of >24,000 genes and EST clusters. The sequences include all rat sequence clusters from Build #34 of the UniGene Datablse (created from GenBank 107/dbEST Nov. 18, 1998) and supplemented with additional annoteted gene sequences from GenBank 110. The chips were hybridized, reacted with phycoerythrin-streptavidin, washed and then incubated with a polyclonal anti-streptavidin antibody coupled to phycoerythrin as an amplification step to aid in the detection of lower abundance transcripts. Following further washing, the expression chip was scanned as above. Analysis of the scanned data was performed using GeneChip software.

Gene Selection

Known or EST gene sequences were first selected as being potentially differentially expressed based on the fold change in hybridization between the naïve animals and either the axotomy or SNI pain models. This was measured as the ratio of the expression level, measured as the intensity of the hybridization signal of the cRNA probe on the microarray for a specific gene, of either SNI or axotomy to naïve. Based on previous studies which demonstrate that the expression of the heat shock protein Hsp27 in increased 1.5 fold after axotomy, a 1.4 fold change in expression in either the axotomy or SNI models relative to naïve was chosen as a numerical cutoff for differential expression. Genes identified as being differentially expressed based on the measurement of an at least 1.4 fold change in expression are shown in tables 1, 2, 3, 4, or 5. Table 1 shows a group of genes which have been previously suggested to exhibit regulated expression in pain models, but which have been evaluated for purposes of the present invention as being differentially expressed by at least 1.4 fold in both a rat axotomy pain model and a SNI pain model relative to the expression level in an animal not subjected to pain. Thus, from the genes and polynucleotides shown in Table 1, only those showing a axotomy/naïve or SNI/naïve ratio of +/−1.4 or greater were identified as being differentially expressed. Tables 2-3 show a number of genes which were identified by the methods of the present invention as being differentially expressed by at least 1.4 fold in an animal subjected to a nerve injury or inflammatory pain model. In addition, the polynucleotides indicated in Table 2, have been further confirmed as being differentially expressed based on triplicate expression analysis (i.e., samples from three different animals hybridized to three different microarrays, wherein samples are obtained from several different animal pain models, and wherein the polynucleotide sequences are differentially expressed by at least 1.2 fold, with a significance of p<0.05 in at least one pain model). Table 4 shows a group of genes which exhibit an at least 1.4 fold increase in expression in the inflammation pain model. Table 5 shows a group of genes which exhibit an at least 1.4 fold decrease in expression in the inflammation pain model. The data in Tables 1, 3, 4, and 5 represent the average hybridization measurements obtained from at least two rat gene chips.

Genes identified as being differentially expressed based on an at least 1.4 fold change in expression were then screened by Northern analysis to verify differential expression.

Northern Analysis

For each gene suggested to be differentially expressed based on the microarray data, RT-PCR was performed on DRG total RNA obtained from the axotomy, SNI and naïve animal groups as described above. RT-PCR was performed according to techniques known in the art. The cDNA fragments generated in this manner were subsequently cloned into a PCRII vector using the TA cloning kit (Invitrogen). The identity of each fragment was verified by sequencing in each direction from the T3 and T7 polymerase sites present in the cloning vector. The cDNA molecules produced in this manner were then used to produce ³²P-labeled cDNA probes using the Prime-It kit from Stratagene. Subsequently, 5 to 10 μg of total RNA isolated from axotomy, SNI and naïve DRGs were separated on an agarose/formaldehyde gel in 1× MOPS buffer. Following staining with ethidium bromide and visualization under ultra violet light to determine the integrity of the RNA, the RNA is hydrolyzed by treatment with 0.05M NaOH/1.5MNaCl followed by incubation with 0.5M Tris-Cl (pH 7.4)/1.5M NaCl. The RNA is transferred to a commercially available nylon or nitrocellulose membrane (e.g. Hybond-N membrane, Amersham, Arlington Heights, Ill.) by methods well known in the art (Ausubel et al., supra, Sambrook et al., supra). Following transfer and UV cross linking, the membrane is hybridized with a ³²P-labeled cDNA probe, having a sequence complementary to the mRNA sequences identified as being differentially expressed by microarray analysis, in hybridization solution (e.g. in 50% formamide/2.5% Denhardt's/100-200 mg denatured salmon sperm DNA/0.1% SDS/5×SSPE) overnight at 65° C. The hybridization conditions can be varied as necessary as described in Ausubel et al., supra and Sambrook et al., supra. Following hybridization, the membrane is washed at room temperature in 2×SSC/0.1% SDS, at 42° C. in 1×SSC/0.1% SDS, at 65° C. in 0.2×SSC/0.1% SDS, and exposed to film overnight with an intensifying screen at −80° C. The stringency of the wash buffers can also be varied depending on the amount of background signal (Ausubel et al., supra). The film was subsequently developed and the intensity bands corresponding to the radiolabeled probe hybridized to RNA were quantified using methods known to those of skill in the art, for example, by digitizing the film and analyzing the band intensity with a computer software program such as NIH Image (NIH, Bethesda, Md.).

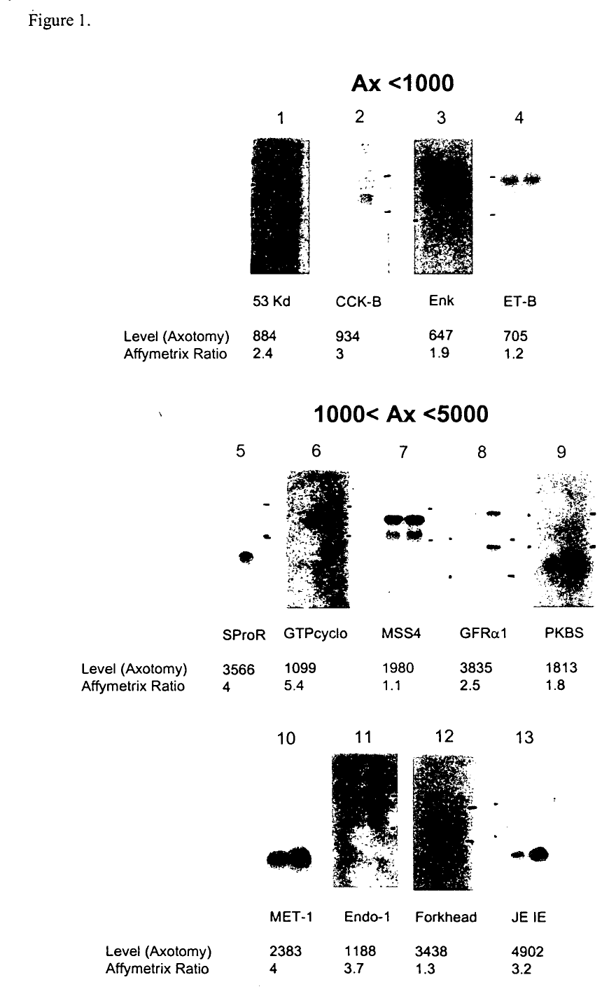

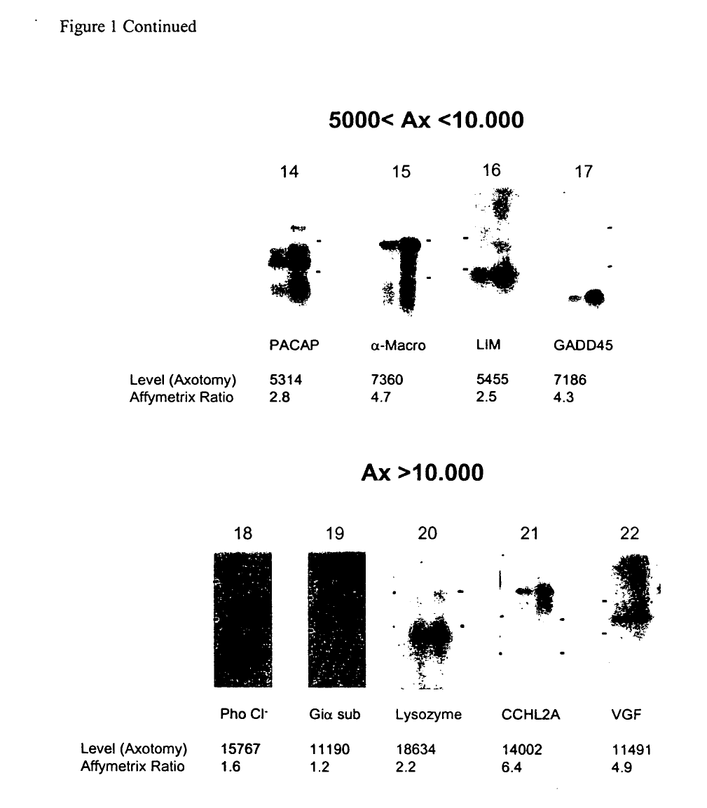

FIG. 1 shows an example of Northern data which confirms the differential expression, or lack thereof, of 22 genes which were initially screened by microarray analysis of cRNA samples obtained from animals subjected to the axotomy pain model. Table 8 shows the correlation of the data obtained from the microarray analysis for these 22 genes and the data obtained by Northern analysis.

### Example 2

### Verification by In Situ Hybridization

In addition to verification of differential expression using Northern analysis, the present invention provides that the differential expression of genes in an animal subjected to pain may be confirmed using in situ hybridization.

In situ hybridization is carried out on fresh frozen, 5 μm thick sections of the dorsal root ganglia from spinal levels L4-L5 obtained from animals subjected to pain, using isotopically-labeled probes. Forty-eight base pair oligonucleotide probes are designed to have 50% G-C content and be complementary to and selective for the desired mRNA. Probes are 3′-end labeled with ³⁵S or ³³P-dATP using a terminal transferase reaction and purified through a spin column. Hybridization is carried out such that homologies greater than 90% are required for detection of transcripts (Dagerlind et al., '92 Histochemistry 98:39). Generally, slides are brought to room-temperature and covered with a hybridization solution (50% formamide, 1× Dendhardt's solution, 1% sarcosyl, 10% dextran sulphate, 0.02M phosphate buffer, 4×SSC, 200 nM DTT, 500 mg/ml salmon sperm DNA) containing 107 cmp/ml of labeled probe. Slides are incubated in a humidified chamber at 43° C. for 14-18 hours, then washed 4×15 min in 1×SSC at 55° C. In the final rinse, slides are brought to room temperature, washed in dH2O, dehydrated in ethanol and air dried.

Autoradiograms are generated by dipping slides in NTB2 nuclear track emulsion and storing the dark at 4° C. Prior to conventional developing and fixation, sections are allowed to expose for 1-12 weeks, depending on the abundance of transcript. Unstained tissue is viewed under darkfield conditions using a fiber-optic darkfield stage adapter (MVI), while stained tissue is examined under brightfield conditions. Control experiments are conducted to confirm the specificity of the oligonucleotide probes. Sections are hybridized with labeled probe, labeled probe with a 1,000-fold excess of cold probe, or labeled probe with a 1,000-fold excess of another, dissimilar cold probe of the same length and similar G-C content.

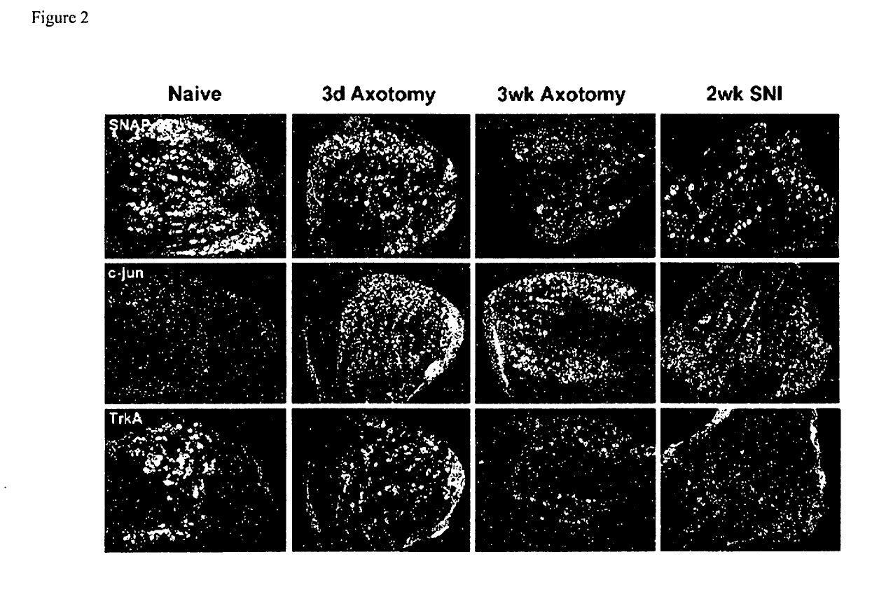

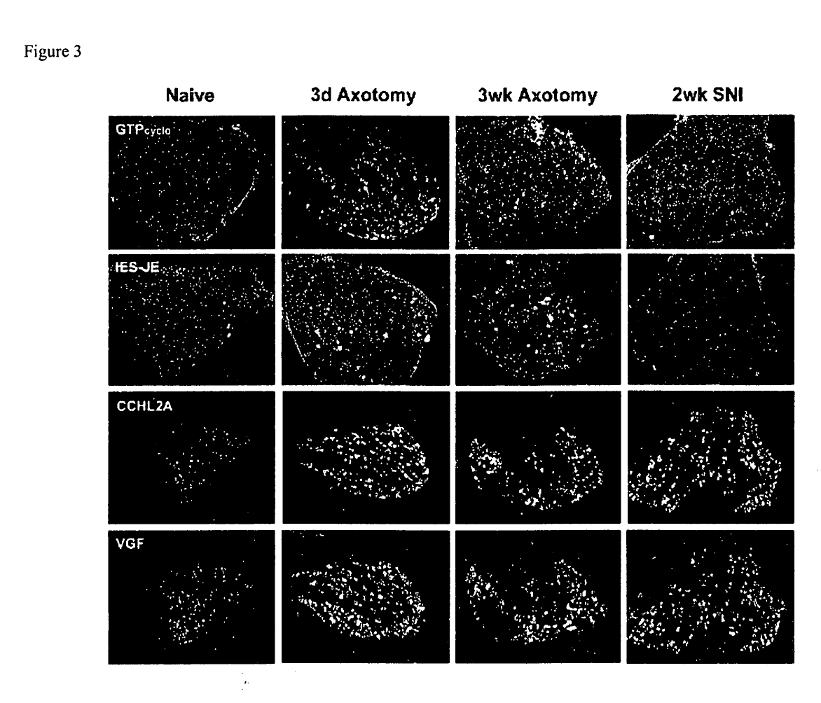

The use of serial, thin sections permits the identification of the same cells in adjacent sections, allowing for comparisons to be made with other markers by in situ hybridization or immunohistochemistry. The technique unlike non-isotopic in situ using digoxygenin labeled riboprobes is suited to screening more than detailed analysis of co-expression of multiple markers. FIGS. 2 and 3 show the results of in situ hybridization verification of the differential expression of five genes (GTPcyclo, IES-JE, CCHL2A, VGF, SNAP, c-jun, and TrkA) in the dorsal root ganglia of a rat axotomy pain model and a rat spared nerve injury pain model.

### Example 3

### Verification of Differential Expression by Real-Time PCR

In addition to verification of differential expression by Northern analysis or in situ hybridization, the differential expression of genes in an animal subjected to pain may be verified using real-time PCR and TaqMan® probes. The technique of real-time PCR is well known in the art (see, for example, U.S. Pat. Nos. 5,691,146; 5,779,977; 5,866,336; and 5,914,230). cDNA samples obtained from a rat axotomy pain model were amplified using primers specific for 19 genes which had previously been examined by microarray analysis and SYBR Green I as the double stranded DNA binding dye. PCR products were generated using an ABI 7700 sequence detection system (Applied Biosystems, Foster City, Calif.). A comparison of the expression level measured by microarray analysis and that obtained by real-time PCR is shown in Table 9. A close correlation can be seen between the differential expression, or lack thereof, of genes examined by microarray analysis and using the Taqman® technique.

### Example 4

### Triplicate Analysis

As described above, a polynucleotide sequence is identified as being differentially regulated in an animal subjected to pain relative to an animal not subjected to the same pain if the sequence is differentially expressed by at least 1.4 fold, and additionally, if the differential expression attains a statistical significance over at least three replicate screens, in at least on pain model, with a p-value of less than 0.05. This example describes how to perform such a statistical analysis, using the axotomy and SNI pain models.

Surgical Procedures.

Adult male Sprague Dawley rats (200-300 g) are anesthetized with halothane. For the sciatic nerve transection (axotomy), the left sciatic nerve is exposed at the mid thigh level, ligated with 3/0 silk and sectioned distally. The wound is sutured in two layers, and the animals were allowed to recover.

Tissue and RNA Preparation.

Animals are terminally anesthetized with CO₂, the L4 and L5 DRGs rapidly removed, and stored at −80° C. Total RNA is extracted from homogenized DRG samples using acid phenol extraction (TRIzol reagent, Gibco-BRL). RNA concentration is evaluated by A₂₆₀ measurement and quality assessed by electrophoresis on a 1.5% agarose gel. Each RNA sample used for hybridization of each array can be extracted, for example, from rat L4 and L5 DRGs (10 ganglia pooled from 5 animals, per sample).

Microarray Analysis

Affymetrix rat genome U34A oligonucleotide microarrays, representing 8799 known transcripts and expressed sequence tags (ESTs), can be used (Affymetrix, Santa Clara, Calif.). Oligonucleotides are arranged in pairs corresponding to different regions of the target mRNA with multiple probe pairs. Each probe pair consists of a 25 nucleotide perfect match (PM) to the target region coupled with a 25-mer with a single mismatch (MM) at the 13^{th} nucleotide. Transcript abundance is estimated by analysis of signal intensity of the PM/MM pairs. The arrays are hybridized with biotin-labeled CRNA, prepared as per standard Affymetrix protocol. Briefly, total RNA (8 μg) from DRGs was reverse transcribed using an oligo-dT primer coupled to a T7 RNA polymerase binding site. Double-stranded cDNA can be made and biotinylated-cRNA synthesized using T7 polymerase. The cRNA is then hybridized for about 16 hours to an array, followed by binding with a streptavidin-conjugated fluorescent marker, and then incubated with a polyclonal anti-streptavidin antibody coupled to phycoerythrin as an amplification step. Following washing, the chips are scanned with a Hewlett-Packard GeneArray laser scanner and data analyzed using GeneChip software. External standards can be included to control for hybridization efficiency and sensitivity.

Hybridization levels for each species of mRNA detected on the arrays are expressed by intensity (signal) and as present (P), marginal (M) or absent (A) calls, calculated by Affymetrix software (MAS 5.0, α1=0.04 α2=0.06). For calculation of signal values, each array is scaled to a target signal of 2500 across all probe sets, to allow comparison between arrays.

The arrays are grouped for two comparisons: two triplicate sets of naïve data compared with one another, and one triplicate naïve set compared with one triplicate post-axotomy set. The individual naïve arrays included in each triplicate set are picked randomly. A probe set is determined undetected if it received an A call in all of the six arrays involved in the comparison. Detected are Present or Marginal by MAS5.0 in at least one array for each analysis. Mean signal and standard deviation are calculated for each detected probe set. The p-value for rejecting the null hypothesis that the mean signals were equal between the two triplicate sets is calculated using an unpaired, two-tailed t-test for independent samples with unequal variance (Satterthwaite's method). Fold-differences between the mean signals (A and B) in the two triplicate sets is calculated as max(A, B)/min(A, B) with down regulation relative to naïve expressed as negative.

As noted above, a polynucleotide sequence is considered to be differentially expressed according to the present invention if it is differentially expressed by at least 1.4 fold in an animal subjected to pain relative to an animal not subjected to the same pain, and optionally, is also statistically significantly differentially expressed with a p-value of less than 0.05 across at least three replicate expression screens.

### Example 5

### Pain-specific Microarray Construction

A microarray according to the invention was constructed as follows.

cDNA samples obtained from the dorsal root ganglia of either naïve animals or animals which have been subjected to pain are amplified using primers specific for the genes which have been identified as being differentially expressed using the methods described above. PCR products (˜40 ul) in the same 96-well tubes used for amplification, are precipitated with 4 ul (1/10 volume) of 3M sodium acetate (pH 5.2) and 100 ul (2.5 volumes) of ethanol and stored overnight at −20° C. They are then centrifuged at 3,300 rpm at 4° C. for 1 hour. The obtained pellets were washed with 50 ul ice-cold 70% ethanol and centrifuged again for 30 minutes. The pellets are then air-dried and resuspended well in 20 ul 3×SSC overnight. The samples are then deposited either singly or in duplicate onto polylysine-coated slides (Sigma Cat. No. P0425) using a robotic GMS 417 arrayer (Genetic MicroSystems, MA). The boundaries of the DNA spots on the microarray are marked with a diamond scriber. The invention provides for arrays wherein 10-20,000 PCR products are spotted onto a solid support to prepare an array.

The arrays are rehydrated by suspending the slides over a dish of warm particle free ddH₂O for approximately one minute (the spots will swell slightly but not run into each other) and snap-dried on a 70-80° C. inverted heating block for 3 seconds. DNA is then UV crosslinked to the slide (Stratagene, Stratalinker, 65 mJ—set display to “650” which is 650×100 UJ). The arrays are placed in a slide rack. An empty slide chamber is prepared and filled with the following solution: 3.0 grams of succinic anhydride (Aldrich) is dissolved in 189 ml of 1-methyl-2-pyrrolidinone (rapid addition of reagent is crucial); immediately after the last flake of succinic anhydride dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are then stored in the slide box at room temperature until use.

### Example 6

### Therapeutic Agent Screening

A candidate agent that increases or decreases the expression of a polynucleotide sequence that is differentially expressed in the sensory neurons of an animal subjected to pain is screened according to the following method.

An animal that has been subjected to pain is treated with a candidate agent for varying amounts of time. Typically an animal is treated by systemic administration of a candidate agent, such as by intravenous administration, on a hourly, daily, or weekly dosing schedule. Following administration, the animals are killed, and the dorsal root gangila are removed and used to prepare cRNA samples as described above. The cRNA samples are then hybridized to a pain-specific microarray, constructed according to the method described above. The hybridization of the cRNA samples to the microarray can be used to determine the level of expression of the genes in the animal subjected to pain which correspond to the differentially expressed genes comprising the microarray. Thus any changes in the predicted differential expression of a gene in an animal treated with a candidate agent is indicative of that agent being capable of increasing or decreasing the expression of a gene which is known to be differentially expressed in an animal subjected to pain.

### Example 7

### In Vivo Protein Activity Screening

Microarrays can be used to screen in vivo for genes that are regulated in pain as a result of the activity of specific protein signaling molecules. To do this, the changes in gene expression produced in the pain models are compared with the changes in gene expression produced in the same models when a particular signaling molecule is neutralized or inhibited by preventing its synthesis, release, transport, binding to a receptor or activation of a cellular response. Any resultant difference in gene expression profile will represent the contribution of the signaling molecule. Further confirmation can be produced by the administration of the signaling molecule in vivo to see if it induces a change in gene regulation.

Such an analysis has been performed looking at the contribution of the neurotrophin nerve growth factor (NGF) to inflammatory pain. Inflammation is known to produce an increase in NGF at the site of the inflammation and this acts on its high affinity receptor TrkA expressed on sensory neurons to change transcription of NGF-regulated genes in the sensory neuron cell body in the DRG. The pattern of expression of genes after inflammation induced in vivo by intraplantar CFA (at 3, 12 24 hrs and 5 days) was compared with naïve non-inflamed animals to detect inflammation-induced genes. This gene expression profile was then compared with arrays produced from RNA from inflamed animals treated with a neutralizing anti-NGF antibody. One example of a gene that was upregulated by CFA, but whose level did not increase in CFA animals treated with antiNGF was the NF-kappaB inhibitor alpha (I kappa B). I kappa B alpha was also upregulated 12 and 24 hrs after intraplantar NGF injection showing that it is an NGF regulated inflammatory-induced gene.

| | |
|---|---|
| Affymetrix accession | X63594cds RRRLIF1 |
| #X63594cds_g_at | R.rattus RL/IF-1 mRNA |

| | CFA | NGF | CFA + anti-NGF |
|---|---|---|---|
| Ni | Fold | Fold | Fold |
| 3 h | −1 | | |
| 6 h | 8.5 | | |
| 12 h | 2.1 | 3.5 | −1.8 |
| 24 h | 3.4 | 1.5 | 1.4 |
| 2 d | 1.1 | | |
| 5 d | 1.6 | | |

Affymetrix accession numbers #X63594cds_g_at and X63594cds RRRLIF1 refer to sequences depicted in Table 2.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

LENGTHY TABLE
The patent application contains a lengthy table section. A copy of the table is available in electronic form from the USPTO web site (http://seqdata.uspto.gov/?pageRequest=docDetail&DocID=US20070026409A1) An electronic copy of the table will also be available from the USPTO upon request and payment of the fee set forth in 37 CFR 1.19(b)(3).

1. A composition comprising two or more isolated polynucleotides, wherein each of said two or more isolated polynucleotides is selected from the polynucleotides of Table 11 or a sequence which hybridizes under high stringency conditions thereto.

2. The composition of claim 1, wherein said each of said two or more isolated polynucleotides is differentially expressed in an animal subjected to pain relative to an animal not subjected to said pain by at least 1.2 fold across at least three replicate screens of neuronal tissue of an animal subjected to pain with a P-value of less than 0.05.

3. The composition of claim 1, wherein said each of said two or more isolated polynucleotides is differentially expressed by at least 1.4 fold in the neuronal tissue of an animal subjected to pain relative to said animal not subjected to said pain.

4. The composition of claim 1, wherein said each of said two or more isolated polynucleotides is differentially expressed by at least 2 fold in the neuronal tissue of an animal subjected to pain relative to said animal not subjected to said pain.

5. The composition of claim 1, wherein said neuronal tissue is selected from the group consisting of sensory neurons of the dorsal root ganglion, and dorsal horn neurons.

6. A plurality of vectors each comprising an isolated polynucleotide, wherein each of said isolated polynucleotides is selected from Table 11, or a sequence which hybridizes under high stringency conditions thereto.

7. A plurality of viral vectors each comprising an isolated polynucleotide, wherein each of said isolated polynucleotides is selected from Table 11, or a sequence which hybridizes under high stringency conditions thereto.

8. A host cell comprising the vectors of claim 6 or 7.

9. An array consisting essentially of:
(a) a plurality of polynucleotide members, wherein each of said polynucleotide members is selected from Table 11; and
(b) a solid substrate, wherein each polynucleotide member has a unique position on said array and is stably associated with said solid substrate.

10. An array comprising:
(a) a plurality of polynucleotide members, wherein each of said polynucleotide members is selected from Table 11, and wherein said plurality of polynucleotide members are obtained from neuronal tissue obtained from at least two different species of animal; and
(b) a solid substrate, wherein each polynucleotide member obtained from each of said two different species has a unique position on said array and is stably associated with said solid substrate.

11. The array of claim 9 or 10, wherein said plurality of polynucleotide members is differentially expressed by at least 1.2 fold across at least three replicate screens of neuronal tissue of an animal subjected to pain with a P-value of less than 0.05 relative to an animal not subjected to said pain.

12. The array of claim 9 or 10, wherein said plurality of polynucleotide members is differentially expressed by at least 1.4 fold in the neurons of said animal subjected to pain relative to an animal not subjected to said pain.

13. The array of claim 9 or 10, wherein said array comprises from 10 to 20,000 polynucleotide members.

14. The array of claim 9 or 10, further comprising negative and positive control sequences and quality control sequences selected from the group consisting of cDNA sequences encoded by housekeeping genes, plant gene sequences, bacterial sequences, PCR products and vector sequences.

15. A method of identifying an agent that increases or decreases the expression of a polynucleotide sequence that is differentially expressed in neuronal tissue of a first animal which is subjected to pain comprising:
(a) administering said agent to said first animal;
(b) hybridizing nucleic acid isolated from one or more sensory neurons of said first and a second animal to the array of claim 9 or 10; and
(c) measuring the hybridization of said nucleic acid isolated from said neuronal tissue of said first and second animal to said array; wherein an increase in hybridization of said nucleic acid from said first animal to one or more nucleic acid members of said array relative to hybridization of said nucleic acid from a second animal which is subjected to pain but to which is not administered said agent to one or more nucleic acid members of said array identifies said agent as increasing the expression of said polynucleotide sequence, and wherein a decrease in hybridization of said nucleic acid from said first animal to one or more nucleic acid members of said array relative to the hybridization of said nucleic acid from second animal to one or more nucleic acid members of said array identifies said agent as decreasing the expression of said polynucleotide sequence.

16. The method of claim 15, further comprising the step of verifying the increase or decrease in said hybridization by a molecular procedure selected from the group consisting of Northern analysis, in situ hybridization, and PCR.

17. The method of claim 15, further comprising the step of labeling the nucleic acid sample isolated from said first and second animal with a detectable label prior to said hybridization to said array.

18. The method of claim 17, wherein the nucleic acid sample isolated from said first animal is labeled with a different detectable label than the nucleic acid sample isolated from said second animal.

19. A method for identifying a compound which regulates the expression of a polynucleotide sequence which is differentially expressed in an animal subjected to pain, comprising:
(a) providing a cell comprising and capable of expressing one or more of the differentially expressed polynucleotide sequences shown in Table 11;
(b) contacting said cell with a candidate compound; and
(c) measuring the expression of said one or more of the polynucleotide sequences shown in Table 11, wherein an increase or decrease in the expression of said one or more of the polynucleotide sequences shown in Table 11 of at least 10% is indicative of regulation of said differentially expressed polynucleotide sequence.

20. A method for identifying a compound which increases or decreases pain in an animal, comprising:
(a) providing a cell comprising one or more of the polypeptides shown in Table 11;
(b) contacting said cell with a candidate compound; and
(c) measuring the activity of said one or more polypeptides, wherein an increase or decrease of the activity of said one or more polypeptides of at least 10% relative to the activity of said one or more polypeptides in said cell, wherein the cell is not contacted with the candidate compound, identifies said candidate compound as a compound which increases or decreases pain in an animal.

21. The method of claim 20, wherein said candidate compound is selected from the group consisting of small molecule, protein, RNAi, and antisense.

22. The method of claim 20, wherein said candidate compound is an antibody which binds to said polypeptide.

23. A method for producing a pharmaceutical formulation comprising:
(a) providing a cell comprising said one or more of the polypeptides shown in Table 11;
(b) selecting a compound which regulates the activity of said one or more polypeptides; and
(c) mixing said compound with a carrier.

24. The method of claim 23, wherein said step of selecting comprises the steps of
(a) contacting said cell with a candidate compound; and
(b) measuring the activity of said one or more polypeptides, wherein an increase or decrease of the activity of said one or more polypeptides of at least 10% relative to the activity of said one or more polypeptides in said cell, wherein the cell is not contacted with the candidate compound, identifies said candidate compound as a compound which regulates the activity of said one or more polypeptides

25. A method for identifying a compound which increases or decreases pain in an animal, comprising:
(a) administering a candidate compound to an animal comprising one or more of the polypeptides shown in Table 11; and
(b) measuring the activity of said one or more polypeptides wherein an increase or decrease of the activity of said polypeptide of at least 10% relative to the activity of said one or more polypeptides in an animal to which the candidate compound is not administered, identifies said candidate compound as a compound which increases or decreases pain in an animal.

26. The method of claim 25, wherein said candidate compound is selected from the group consisting of small molecule, protein, RNAi, and antisense.

27. The method of claim 25, wherein said candidate compound is an antibody which binds to said polypeptide.

28. A method for identifying a small molecule which increases or decreases pain in an animal, comprising:
(a) providing a cell comprising one or more of the polypeptides indicated in Table 11;
(b) generating a small molecule library;
(c) providing a candidate small molecule, selected from said library;
(d) contacting said cell with said candidate small molecule; and
(e) measuring the activity of said one or more polypeptides, wherein an increase or decrease of the activity of said one or more polypeptides of at least 10% relative to the activity of said one or more polypeptides in said cell, wherein the cell is not contacted with the candidate small molecule, identifies said candidate small molecule as a small molecule which increases or decreases pain in an animal.

29. The method of claim 28, wherein said small molecule library comprises components selected from the group consisting of heterocyclics, aromatics, alicyclics, aliphatics, steroids, antibiotics, enzyme inhibitors, ligands, hormones, alkaloids, opioids, terpenes, porphyrins, toxins, and catalysts, and combinations thereof.

30. A method for identifying a compound useful in the treatment of pain, comprising:
(a) providing a host cell comprising a vector comprising one or more of the polynucleotides identified in Table 11;
(b) maintaining said host cell under conditions which permit the expression of said one or more polynucleotides;
(c) selecting a compound which regulates the activity of a polypeptide encoded by said one or more polynucleotides;
(d) administering said compound to an animal subjected to pain; and
(e) measuring the level of pain in said animal, wherein a decrease in the level of pain in said animal of at least 10%, identifies said compound as being useful for treating pain.

31. The method of claim 30, wherein said step of selecting includes the steps of
(a) contacting said cell with a candidate compound; and
(b) measuring the activity of the polypeptide encoded by said one or more polynucleotides, wherein an increase or decrease of the activity of said polypeptide of at least 10% relative to the activity of said polypeptide in said cell, wherein the cell is not contacted with the candidate compound, identifies said candidate compound as a compound which regulates the activity of said polypeptide.

32. A method for producing a pharmaceutical formulation comprising:
(a) providing a cell comprising one of more polypeptides as shown in Table 11;
(b) maintaining said cell under conditions which permit the expression of said one or more polynucleotides;
(c) selecting a compound which regulates the activity of a polypeptide encoded by said one or more polynucleotides, wherein an increase or decrease of the activity of said polypeptide of at least 10% relative to the activity of said one or more polypeptides in an animal to which the candidate compound is not administered, identifies said candidate compound as a compound which regulates the activity of said one or more polypeptides;
(d) administering said compound to an animal subjected to pain;
(e) measuring the level of pain in said animal, wherein a decrease in the level of pain in said animal of at least 10%, identifies said compound as decreasing pain; and
(f) mixing said compound with a carrier.

33. A method of treating pain in an animal comprising administering to said animal an antisense polynucleotide capable of inhibiting the expression of one or more of the polynucleotide sequences indicated in Table 11.

34. A method of treating pain in an animal comprising administering to said animal a double stranded RNA molecule wherein one of the strands of said double stranded RNA molecule is identical to a portion of an mRNA transcript obtained from one or more of the polynucleotide sequences indicated in Table 11.

35. A method of treating pain in an animal in need thereof, comprising:
administering a therapeutically effective amount of an antibody which binds to one or more of the polypeptides indicated in Table 11.

36. A method of treating pain in an animal in need thereof, comprising:
administering a therapeutically effective amount of one or more of the polypeptides indicated in Table 11.

37. A method of detecting pain in an animal suspected of having pain comprising:
measuring the amount of one or more of the polynucleotide sequences of Table 11 in said animal, wherein if said amount of said one or more polynucleotides is increased or decreased by at least 1.2 fold across at least three replicate screens and is statistically significant at P<0.05 in said animal compared to the amount of said one or more polynucletoide sequences of Table 11 in an animal not suspected of having pain, then pain is detected in said animal suspected of having pain.

38. The method of claim 37 wherein the amount of said polynucleotide sequence is increased or decreased by at least 1.4 fold.

39. The method of claim 37, wherein said amount of said one or more polynucleotide sequences is measured in one or more sensory neurons.

40. The method of claim 39, wherein said sensory neurons are selected from the group consisting of dorsal horn neurons and dorsal root ganglion neurons.

41. The method of claim 37, wherein said amount of said one or more polynucleotide sequences is measured in a patient sample.

42. A method of detecting pain in an animal suspected of having pain comprising:
measuring the amount of one or more of the polypeptide sequences of Table 11 in said animal, wherein if said amount of said one or more polypeptides is increased or decreased by at least 1.2 fold across at least three replicate screens and is statistically significant at P<0.05 in said animal compared to the amount of said one or more polypeptide sequences of Table 11 in an animal not suspected of having pain, then pain is detected in said animal suspected of having pain.

43. The method of claim 42, wherein said amount of said one or more polypeptide sequences is measured in one or more sensory neurons.

44. The method of claim 43, wherein said sensory neurons are selected from the group consisting of dorsal horn neurons and dorsal root ganglion neurons.

45. The method of claim 42, wherein said amount of said one or more polypeptide sequences is measured in a patient sample.

46. The method of claim 42, wherein the amount of said polypeptide sequence is increased or decreased by 1.4 fold.
